(19) United States
(12) Patent Application Publication
(10) Pub. No.: US 2020/0163804 A1
(43) Pub. Date: May 28, 2020

(54) Absorbent Article Comprising A Three-Dimensional Substrate

(71) Applicant: The Procter & Gamble Company, Cincinnati, OH (US)

(72) Inventors: Rodrigo Rosati, Frankfurt Am Main (DE); Jill Marlene Orr, Liberty Township, OH (US); Adrien Grenier, Frankfurt Am Main (DE); James T. Knapmeyer, Cincinnati, OH (US); John B. Strube, Okeana, OH (US); Aniruddha Chatterjee, Kelkheim (DE); Carsten Kreuzer, Hofheim (DE)

(21) Appl. No.: 16/775,540

(22) Filed: Jan. 29, 2020

Related U.S. Application Data
(63) Continuation of application No. 15/242,985, filed on Aug. 22, 2016.
(60) Provisional application No. 62/213,656, filed on Sep. 3, 2015.

(51) Int. Cl. A61F 13/15; A61F 13/539; A61F 13/534; A61F 13/513; A61F 13/512; A61F 13/537; A61F 13/511

(52) U.S. Cl. CPC A61F 13/15203; A61F 13/539; A61F 13/534; A61F 13/51394; A61F 2013/530007; A61F 13/53743; A61F 13/5116; A61F 13/51104; A61F 13/5125

(57) ABSTRACT

An absorbent article having a longitudinal axis, a transverse axis, a topsheet; a backsheet; an acquisition layer disposed between the topsheet and backsheet; first and second absorbent layers is described. The topsheet and acquisition layer form a three dimensional substrate having a first surface and an opposing second surface, a plurality of recesses and a plurality of land areas, wherein each recess is separated from one another by at least one land area, and wherein each of the plurality of recesses has an opening at a base of the recess, and side walls extending from the second surface to a distal portion. The first absorbent layer is disposed between the acquisition layer and backsheet and has a mixture of pulp and synthetic fibers. The second absorbent layer is disposed between the first absorbent layer and backsheet and has super absorbent polymer attached to a core wrap.

### FIELD OF THE INVENTION

An absorbent article for personal hygiene comprising a three-dimensional substrate, an acquisition layer, first absorbent layer and second absorbent layer, and a backsheet are provided.

### BACKGROUND OF THE INVENTION

An absorbent article typically comprises a topsheet, a backsheet, and an absorbent core disposed between the topsheet and the backsheet. The absorbent article further includes an acquisition layer and optionally a distribution layer. The acquisition layer is able to receive the liquid bodily exudates from the topsheet in order to temporary store them. Then, the distribution layer can receive the liquid bodily exudates from the acquisition layer and distribute and transfer them to the absorbent core in order to make efficient the use of the absorbent core. Such absorbent articles exhibit satisfactory fluid handling properties.

Three-dimensional topsheets have been developed; see for example U.S. Patent application US 2014/0121625 A1.

There still remains a need to further improve the fluid-handling properties of these three-dimensional topsheets when subjected to several gushes of bodily exudates.

There is a need to develop an absorbent article comprising a skin facing layer having a three-dimensional structure which can provide improved fluid handling properties e.g. less rewet on the skin facing layer, while the physical and perceptional comfort of the wearer are still met.

There is a need to provide a skin facing layer having a three-dimensional structure in order to reduce the contact of the liquid bodily exudates with the skin of the wearer. Also, the skin facing layer shall provide softness/cushiness feeling for the caregiver and the wearer.

There is also a need to improve the dryness and absorption perception of the absorbent article to the caregiver. A skin facing layer having a three-dimensional structure in combination with graphics or visual indicia can help the dryness and absorption perception of an absorbent article to the caregiver.

### BRIEF DESCRIPTION OF THE DRAWINGS

While the specification concludes with claims particularly pointing out and distinctly claiming the present invention, it is believed that the same will be better understood from the following description read in conjunction with the accompanying drawings in which:

FIG. 1 is an absorbent article in the form of a diaper comprising an example three-dimensional substrate according to the present invention with some layers partially removed;

FIG. 2 is a transversal cross-section of the diaper of FIG. 1;

FIG. 3 is a top view of a portion of a three-dimensional substrate of FIG. 1, in accordance with the present invention;

FIG. 4 is a top perspective view of the portion of the three-dimensional substrate of FIG. 3 in accordance with the present invention;

FIG. 5 is a top view of a portion of a three-dimensional substrate, in accordance with the present invention;

FIG. 6 is a top perspective view of the portion of the three-dimensional substrate of FIG. 5 in accordance with the present invention;

FIG. 7 is an absorbent article in the form of a diaper comprising an example topsheet/acquisition layer laminate as the three-dimensional substrate, and a tissue layer according to the present invention with some layers partially removed;

FIG. 8 is a transversal cross-section of the diaper of FIG. 7;

FIG. 9 is a transversal cross-section of a diaper from FIG. 7, wherein the tissue layer in now positioned between the distribution layer and the absorbent core according to the present invention with some layers partially removed;

FIG. 10 is an absorbent article in the form of a diaper comprising an example topsheet/acquisition layer laminate as the three-dimensional substrate wherein the three-dimensional protrusions of the topsheet/acquisition layer laminate are only formed where the topsheet overlaps the acquisition layer in the topsheet/acquisition layer laminate, according to the present invention with some layers partially removed;

FIG. 11 is an absorbent article in the form of a diaper comprising an example topsheet/acquisition layer laminate with another type of absorbent core according to the present invention with some layers partially removed;

FIG. 12 is a transversal cross-section of a diaper of FIG. 11;

FIG. 13 is a transversal cross-section of the absorbent article of FIG. 11 taken at the same point as FIG. 12 where channels have formed as a result the absorbent article being loaded with liquid bodily exudates;

FIG. 14 is an absorbent article in the form of a diaper comprising an example topsheet/acquisition layer laminate with an acquisition layer positioned in a front region of the absorbent article according to the present invention with some layers partially removed;

FIG. 15 is an absorbent article in the form of a diaper comprising an example topsheet/acquisition layer laminate with an acquisition layer positioned in a rear region of the absorbent article according to the present invention with some layers partially removed;

FIG. 16A is a perspective view of an apparatus comprising a first and second forming member for forming the topsheet/acquisition layer laminate of the present invention;

FIG. 16B is a perspective view of a portion of the first forming member of the apparatus shown in FIG. 16A;

FIG. 16C is a perspective view of the apparatus shown in FIG. 16A, showing the first forming member intermeshing the second forming member;

FIG. 17A is a perspective view of a three-dimensional protrusion of the topsheet/acquisition layer laminate obtained with the apparatus shown in FIG. 16A;

FIG. 17B is a schematic view of a three-dimensional protrusion of the topsheet/acquisition layer laminate obtained with the apparatus shown in FIG. 16A;

FIG. 17C is a schematic view of a three-dimensional protrusion of the topsheet/acquisition layer laminate obtained with the apparatus shown in FIG. 16A;

FIG. 17D is a schematic view of a three-dimensional protrusion of the topsheet/acquisition layer laminate obtained with the apparatus shown in FIG. 16A;

FIG. 17E is a schematic view of a three-dimensional protrusion of the topsheet/acquisition layer laminate obtained with the apparatus shown in FIG. 16A;

FIG. 17F is a schematic view of a three-dimensional protrusion of the topsheet/acquisition layer laminate obtained with the apparatus shown in FIG. 16A;

FIG. 18A is a schematic view of a three-dimensional protrusion of the topsheet/acquisition layer laminate obtained with the apparatus shown in FIG. 16A;

FIG. 18B is a schematic view of a three-dimensional protrusion of the topsheet/acquisition layer laminate obtained with the apparatus shown in FIG. 16A;

FIG. 18C is a schematic view of a three-dimensional protrusion of the topsheet/acquisition layer laminate obtained with the apparatus shown in FIG. 16A;

FIG. 18D is a schematic view of a three-dimensional protrusion of the topsheet/acquisition layer laminate obtained with the apparatus shown in FIG. 16A;

FIG. 18E is a schematic view of a three-dimensional protrusion of the topsheet/acquisition layer laminate obtained with the apparatus shown in FIG. 16A;

FIG. 19 is a perspective view of an apparatus comprising a first and second intermeshing roll for forming the topsheet/acquisition layer laminate of the present invention;

FIG. 20A is a cross-sectional depiction of a portion of the apparatus shown in FIG. 19;

FIG. 20B is a perspective view of a portion of the second intermeshing roll of the apparatus shown in FIG. 19;

FIG. 21A is a schematic view of a three-dimensional protrusion of the topsheet/acquisition layer laminate obtained with the apparatus shown in FIG. 19;

FIG. 21B is a perspective view of a three-dimensional protrusion of the topsheet/acquisition layer laminate shown in FIG. 21A;

FIG. 21C is another perspective view of a three-dimensional protrusion of the topsheet/acquisition layer laminate shown in FIG. 21A;

FIG. 21D is a schematic view of a three-dimensional protrusion of the topsheet/acquisition layer laminate obtained with the apparatus shown in FIG. 19;

FIG. 21E is a schematic view of a three-dimensional protrusion of the topsheet/acquisition layer laminate obtained with the apparatus shown in FIG. 19;

FIG. 21F is a schematic view of a three-dimensional protrusion of the topsheet/acquisition layer laminate obtained with the apparatus shown in FIG. 19;

FIG. 22A is a schematic view of a three-dimensional protrusion of the topsheet/acquisition layer laminate obtained with the apparatus shown in FIG. 19;

FIG. 22B is a schematic view of a three-dimensional protrusion of the topsheet/acquisition layer laminate obtained with the apparatus shown in FIG. 19;

FIG. 22C is a schematic view of a three-dimensional protrusion of the topsheet/acquisition layer laminate obtained with the apparatus shown in FIG. 19;

FIG. 22D is a schematic view of a three-dimensional protrusion of the topsheet/acquisition layer laminate obtained with the apparatus shown in FIG. 19;

FIG. 23 is an enlarged photographic view of a wet-laid three-dimensional fibrous substrate in accordance with the present invention;

FIG. 24 is an example plan view of a wet-laid three-dimensional substrate of a tissue layer in accordance with the present disclosure;

FIG. 25 is a cross-sectional view of the wet-laid three-dimensional fibrous substrate of FIG. 24;

FIG. 26 is another example plan view of a wet-laid three-dimensional fibrous substrate of the tissue layer in accordance with the present disclosure;

FIG. 27 is a cross-sectional view of the wet-laid three-dimensional fibrous substrate of FIG. 26;

FIG. 28 is a plan view of a portion of a molding member of a papermaking belt of the present invention;

FIG. 29 is a plan view of an example of a raised portion of a molding member for making a wet-laid three-dimensional fibrous substrate of the present invention;

FIG. 30 is a cross-sectional view of a network region and a plurality of discrete zones of a wet-laid three-dimensional fibrous substrate as shown using a SEM micrograph;

FIG. 31 is a processed topography image of a network region and a plurality of discrete zones of a wet-laid three-dimensional fibrous substrate as shown using a SEM micrograph;

FIG. 32 illustrates a series of straight line regions of interest, drawn across the network region and discrete zones shown in FIG. 31;

FIG. 33 illustrates a height profile plot along a straight line region of interest, drawn through a topography image, to show several elevation differential measurements; and

FIG. 34 depicts a height profile plot along a straight line region of interest, drawn through a topography image, to show several transition region widths in accordance with the present disclosure.

### DETAILED DESCRIPTION OF THE INVENTION

### Definition of Terms

The term “absorbent article” as used herein refers to disposable products such as diapers, pants or feminine hygiene sanitary napkins and the like which are placed against or in proximity to the body of the wearer to absorb and contain the various liquid bodily exudates discharged from the body. Typically these absorbent articles comprise a topsheet, backsheet, an absorbent core and optionally an acquisition layer and/or distribution layer and other components, with the absorbent core normally placed between the backsheet and the acquisition system or topsheet. The absorbent article of the present invention may be a diaper or pant.

The term “diaper” as used herein refers to an absorbent article that is intended to be worn by a wearer about the lower torso to absorb and contain liquid bodily exudates discharged from the body. Diapers may be worn by infants (e.g. babies or toddlers) or adults. They may be provided with fastening elements.

The term “extensible” as used herein refers to a material, which, upon application of a force, is capable of undergoing an apparent elongation of equal to or greater than at least 100% of its original length along longitudinal and/or transversal axis of the absorbent article at or before reaching the breaking force if subjected to the following test:

The longitudinal and/or transversal axis tensile properties are measured using a method using INDA WSP 110.4 (05) Option B, with a 50 mm sample width, 60 mm gauge length, and 60 mm/min rate of extension.

It may be desirable that a material is capable of undergoing an apparent elongation of equal to or greater than at least 100% or 110% or 120% or 130% up to 200% along longitudinal and/or transversal axis of the absorbent article at or before reaching the breaking force according to the Test Method as set out above.

If a material is capable of undergoing an apparent elongation of less than 100% of its original length if subjected to the above described test, it is “non-extensible” as used herein.

The term “three-dimensional substrate” as used herein refers to either a substrate comprising three-dimensional protrusions and forming a portion or all of a topsheet. Alternatively, a three-dimensional substrate is a topsheet/acquisition layer laminate comprising three-dimensional protrusions.

The term “topsheet/acquisition layer laminate” as used herein refers to an intimate combination of a topsheet with an acquisition layer, both disposed in a face to face relationship. The topsheet has a first and second surface. The first surface of the topsheet is facing towards the body of the wearer when the absorbent article is in use. The acquisition layer is facing the backsheet or the optional distribution layer. The topsheet and the acquisition layer can have undergone a simultaneous and joint mechanical deformation while the topsheet and the acquisition layer are combined with each other. The topsheet/acquisition layer laminate comprises deformations forming three-dimensional protrusions. In the topsheet/acquisition layer laminate, the topsheet and acquisition layer may be in an intimate contact with each other.

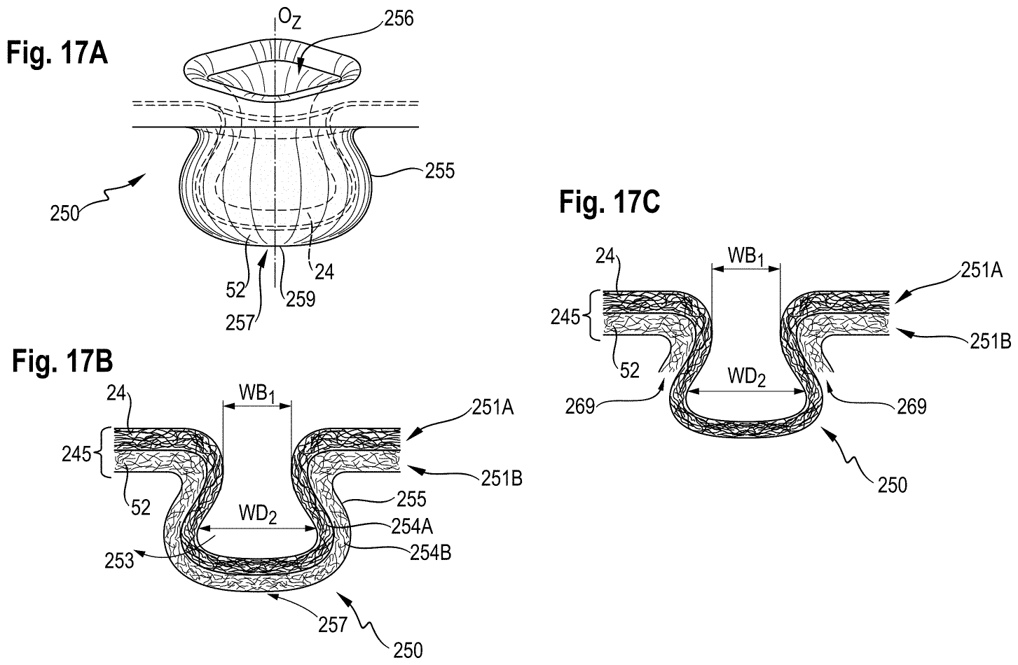

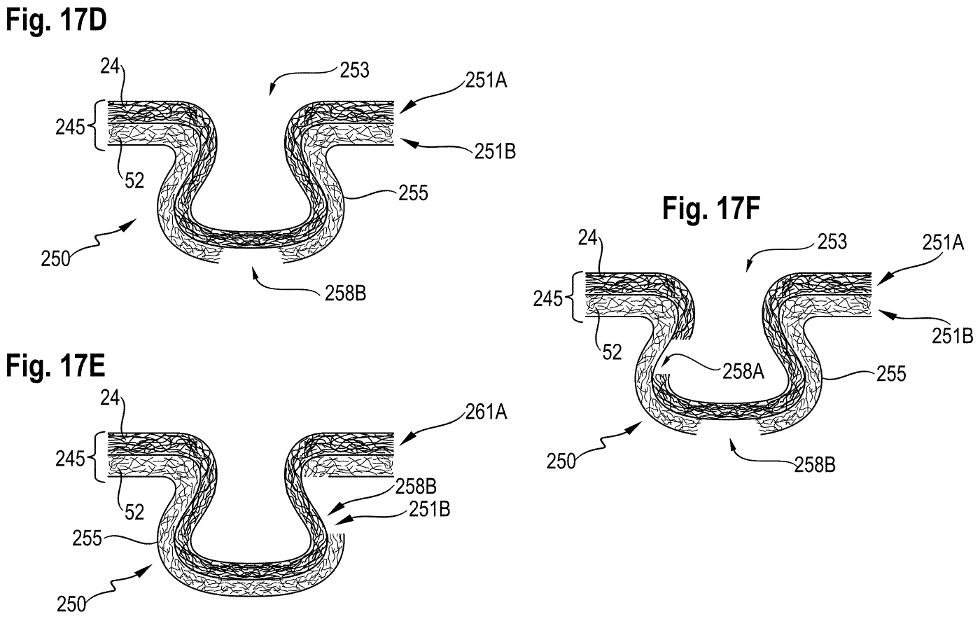

The topsheet/acquisition layer laminate may be formed by nesting together the topsheet and acquisition layer, wherein the three-dimensional protrusions of the topsheet coincide with and fit together with the three-dimensional protrusions of the acquisition layer, as shown in FIGS. 17B, 18A, 21A and 22A. The topsheet and acquisition layer may be both extensible such that the topsheet and acquisition layer are able to stretch.

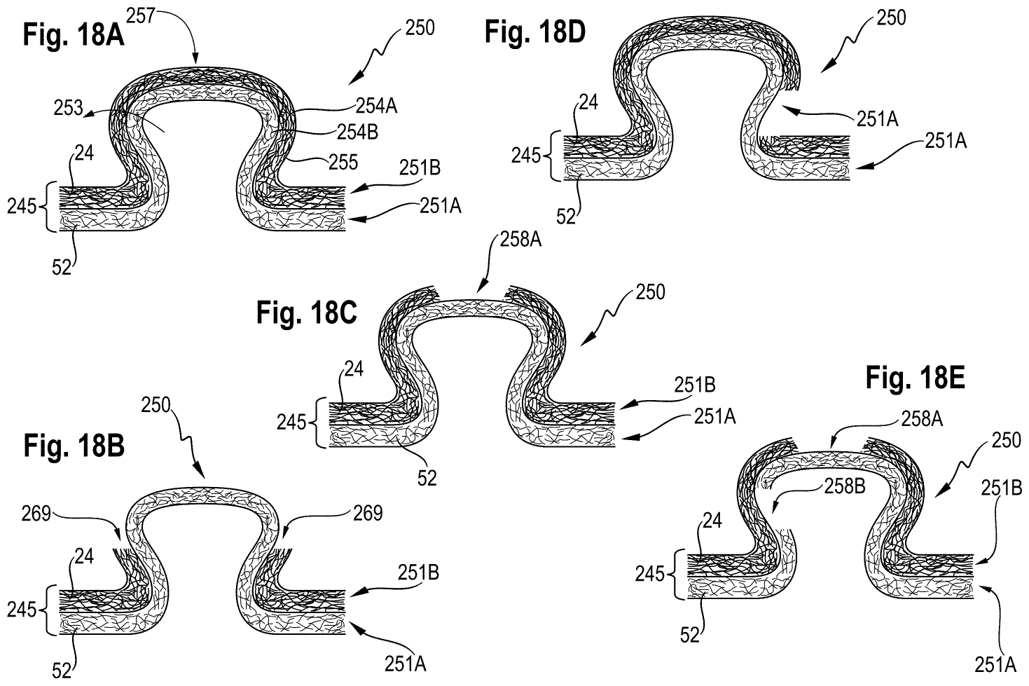

Alternatively or in addition to what has been set out above, the topsheet/acquisition layer laminate may be formed by interrupting one of the topsheet or acquisition layer such that the three-dimensional protrusions of the respective other non-interrupted topsheet or acquisition layer interpenetrate the interrupted topsheet or acquisition layer, as shown in FIGS. 17C and 18B.

In still another alternative or in addition to what has been set out above, the topsheet/acquisition layer laminate may be formed by interrupting one of the topsheet or acquisition layer in the area of the three-dimensional protrusions of the topsheet/acquisition layer laminate such that the three-dimensional protrusions of the respective other non-interrupted topsheet or acquisition layer at least partially fit together with the three-dimensional protrusions of the interrupted topsheet or acquisition layer, as shown in FIGS. 17D, 17E, 18C, 18D, 21D, 21E, 22B and 22C.

In another alternative or in addition to what has been set out above, the topsheet/acquisition layer laminate may be formed by interrupting the topsheet and acquisition layer in the area of the three-dimensional protrusions of the topsheet/acquisition layer laminate and the three-dimensional protrusions of the topsheet coincide with and fit together with the three-dimensional protrusions of the acquisition layer. If the topsheet and acquisition layer comprise interruptions in the area of the three-dimensional protrusions, the interruptions in the topsheet in the area of the three-dimensional protrusions of the topsheet/acquisition layer laminate will not coincide with the interruptions in the acquisition layer in the area of the three-dimensional protrusions of the topsheet/acquisition layer laminate, as shown in FIGS. 17F, 18E, 21F and 22D.

The terms “interruptions”, as used herein, refer to holes formed in the topsheet and/or acquisition layer during the formation of the topsheet/acquisition layer laminate, and does not include the pores and interstices between fibers typically present in nonwovens.

The term “mechanically deforming and combining” as used herein means that the topsheet and acquisition layer are put in a face to face relationship and can be simultaneously mechanically deformed between a first and second roll and intimately combined at the same time. The mechanical deformation of the topsheet and acquisition layer depends on the process, the required apparatus but also on the properties of the topsheet and acquisition layer, i.e. apparent elongation of the fibers, fiber mobility, ability to deform and stretch in the area where the three-dimensional protrusions of the topsheet/acquisition layer laminate are formed, ability to undergo plastic deformation which sets after existing the first and second roll, or springing partially back due to elastic recovery.

The mechanical deformation may comprise engaging the topsheet and the acquisition layer together between a first and second forming member such that a plurality of deformations comprising three-dimensional protrusions are obtained. Alternatively, the mechanical deformation may comprise engaging the topsheet and the acquisition layer together between a first and second intermeshing rolls such that a plurality of deformations comprising three-dimensional protrusions are obtained.

The term “machine direction” or “MD” as used herein means the path that material, such as a web, follows through a manufacturing process.

The term “cross-machine direction” or “CD” as used herein means the path that is perpendicular to the machine direction in the plane of the web.

The term “wet-laid” as used herein is a process step in papermaking. In the wet-laid process, pulp fibers (wood or non-wood) are first mixed with chemicals and water to obtain a uniform dispersion called a slurry at very high dilutions of 0.01 percent weight to 0.5 percent weight of the fibers. The slurry is then deposited on a moving foraminous member (or wire screen) where the excess water is drained off, leaving the fibers randomly laid in a uniform substrate, which is then bonded and finished as required.

The term “wet-formed” as used herein refers to wet-laid fibrous substrates that have a three-dimensional structure imparted to them by the papermaking process of the present invention.

The term “cellulosic fiber” as used herein refers to natural fibers which typically are wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web.

The term “substrate” as used herein refers to an individual, self-sustaining, integral web that may comprise one or more layers.

The term “fibrous substrate” as used herein refers to an individual, self-sustaining, integral web comprising pulp fibers. The fibrous substrate may comprise two or more stratified non-self-sustaining hardwood and/or softwood portions.

The term “dry-laid fiber” as used herein means fibers which have been provided in a fluid medium which is gaseous (air).

The term “web” as used herein means a material capable of being wound into a roll. Webs may be nonwovens.

The term “intensive properties” as used herein are properties which do not have a value dependent upon an aggregation of values within the plane of the fibrous substrate. A common intensive property is an intensive property possessed by more than one region or zone. Such intensive properties of the fibrous substrate comprise, without limitation, average density, basis weight, elevation, dry caliper, and opacity. For example, if average density is a common intensive property of two differential regions, a value of the average density in one region or zone can differ from a value of the average density in the other region or zone. Regions or zones (such as, for example, a first region and a second region) are identifiable areas distinguishable from one another by distinct intensive properties.

The term “substantially continuous” regions as used herein refers to an area within which one can connect any two points by an uninterrupted line running entirely within that area throughout the line's length. That is, the substantially continuous region has a substantial “continuity” in all directions parallel to the first plane and is terminated only at edges of that region. The term “substantially,” in conjunction with continuous, is intended to indicate that while an absolute continuity is preferred, minor deviations from the absolute continuity may be tolerable as long as those deviations do not appreciably affect the performance of the fibrous substrates (or a papermaking belt) as designed and intended.

The term “substantially semi-continuous” regions as used herein refer to an area which has “continuity” in all, but at least one, directions parallel to the first plane, and in which area one cannot connect any two points by an uninterrupted line running entirely within that area throughout the line's length. The semi-continuous framework may have continuity only in one direction parallel to the first plane. By analogy with the continuous region, described above, while an absolute continuity in all, but at least one, directions is preferred, minor deviations from such a continuity may be tolerable as long as those deviations do not appreciably affect the performance of the fibrous substrate.

The term “discrete zones” as used herein refer to regions that are discontinuous and separated from other areas in all directions parallel to the first plane.

The term “papermaking belt” as used herein refers to a structural element that is used as a support for the fiber or filaments that may be deposited thereon during a process of making a fibrous substrate, and as a forming unit to form a desired microscopical geometry of a fibrous substrate. The papermaking belt may comprise any element that has the ability to impart a three-dimensional pattern to the fibrous substrate being produced thereon, and includes, without limitation, a stationary plate, a belt, a cylinder/roll, a woven fabric, and a band.

The term “caliper” as used herein refers to the thickness of a substrate under a defined load, e.g. at 2.06 kPa.

The term “basis weight” as used herein refers to the weight per unit area of a sample reported in gsm and is measured according to the Basis Weight Test Method described herein.

The term “absorbent core” as used herein refers to a component, which is placed or is intended to be placed within an absorbent article and which comprises an absorbent material enclosed in a core wrap. The term “absorbent core” does not include an acquisition or distribution layer or any other component of an absorbent article which is not either an integral part of the core wrap or placed within the core wrap. The absorbent core is typically the component of an absorbent article which comprises all, or at least the majority of, superabsorbent polymer and has the highest absorbent capacity of all the components of the absorbent article.

The term “substantially free of absorbent material” or “substantially absorbent material free” as used herein means that the basis weight of the absorbent material in the substantially absorbent material free areas is at least less than 10%, in particular less than 5%, or less than 2%, of the basis weight of the absorbent material in the rest of the absorbent core.

The term “superabsorbent polymers” (herein abbreviated as “SAP”) as used herein refer to absorbent materials which are cross-linked polymeric materials that can absorb at least 10 times their weight of an aqueous 0.9% saline solution as measured using the Centrifuge Retention Capacity (CRC) test (EDANA method WSP 241.2-05E). The SAP of the invention may in particular have a CRC value of more than 20 g/g, or more than 25 g/g, or from 20 to 50 g/g, or from 20 to 40 g/g, or 25 to 35 g/g. The SAP useful in the invention includes a variety of water-insoluble, but water-swellable polymers capable of absorbing large quantities of liquid bodily exudates.

The term “a majority of the three-dimensional protrusions” as used herein means that more than 50% or more than 60% or more than 70% or more than 80% or more than 90% or more than 95% or more than 98% of the three-dimensional protrusions in the three-dimensional substrate of the absorbent article.

The term “joined to” as used herein encompasses configurations in which an element is directly secured to another element by affixing the element directly to the other element; and configurations in which the element is indirectly secured to the other element by affixing the element to intermediate member(s) which in turn are affixed to the other element. The term “joined to” encompasses configurations in which an element is secured to another element at selected locations, as well as configurations in which an element is completely secured to another element across the entire surface of one of the elements. The term “joined to” includes any known manner in which elements can be secured including, but not limited to mechanical entanglement.

The term “joined adjacent to the transversal edges” as used herein means that when a first and/or second transversal edge of a first layer is/are joined adjacent to a first and/or second transversal edges of a second layer, the first and/or second transversal edge of the first layer are disposed within an area spaced inboard from the first and/or second transversal edge of the second layer. The area has a width which is from 1 to 30% of the width of the second layer.

“Comprise,” “comprising,” and “comprises” are open ended terms, each specifies the presence of the feature that follows, e.g. a component, but does not preclude the presence of other features, e.g. elements, steps, components known in the art or disclosed herein. These terms based on the verb “comprise” should be read as encompassing the narrower terms “consisting essential of” which excludes any element, step or ingredient not mentioned which materially affect the way the feature performs its function, and the term “consisting of” which excludes any element, step, or ingredient not specified. Any preferred or example embodiments described below are not limiting the scope of the claims, unless specifically indicated to do so. The words “typically”, “normally”, “advantageously” and the likes also qualify features which are not intended to limit the scope of the claims unless specifically indicated to do so.

### General Description of the Absorbent Article 20

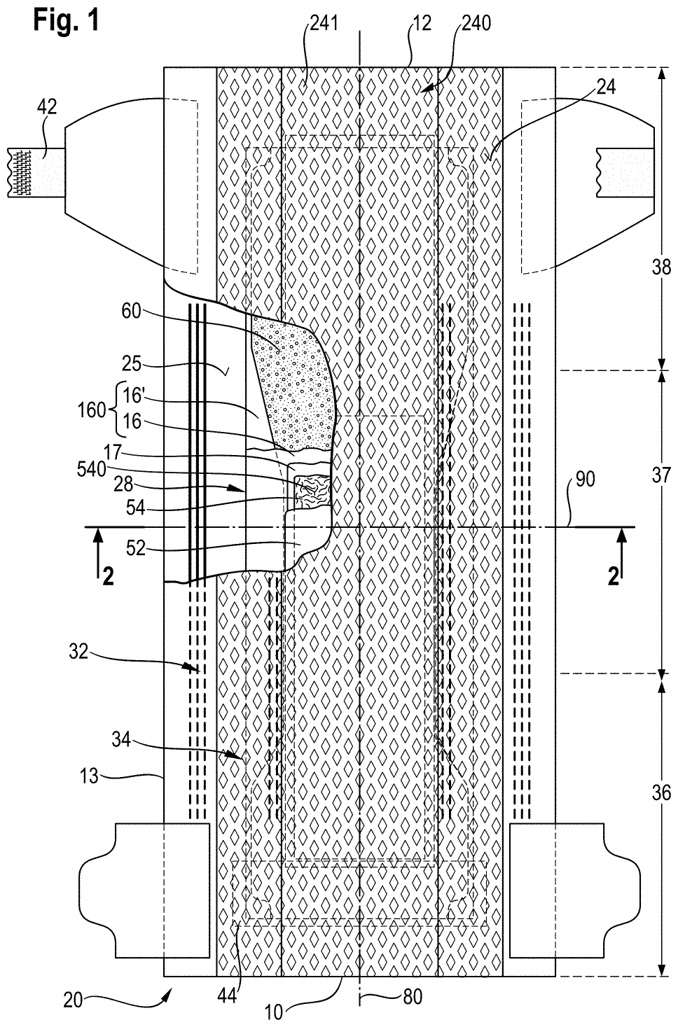

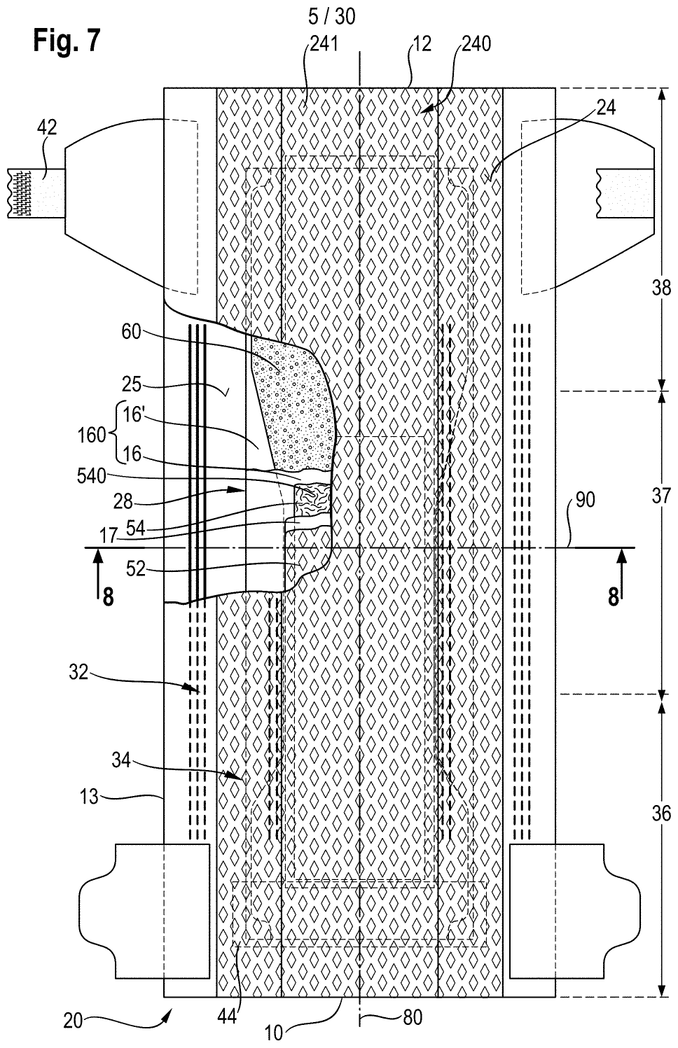

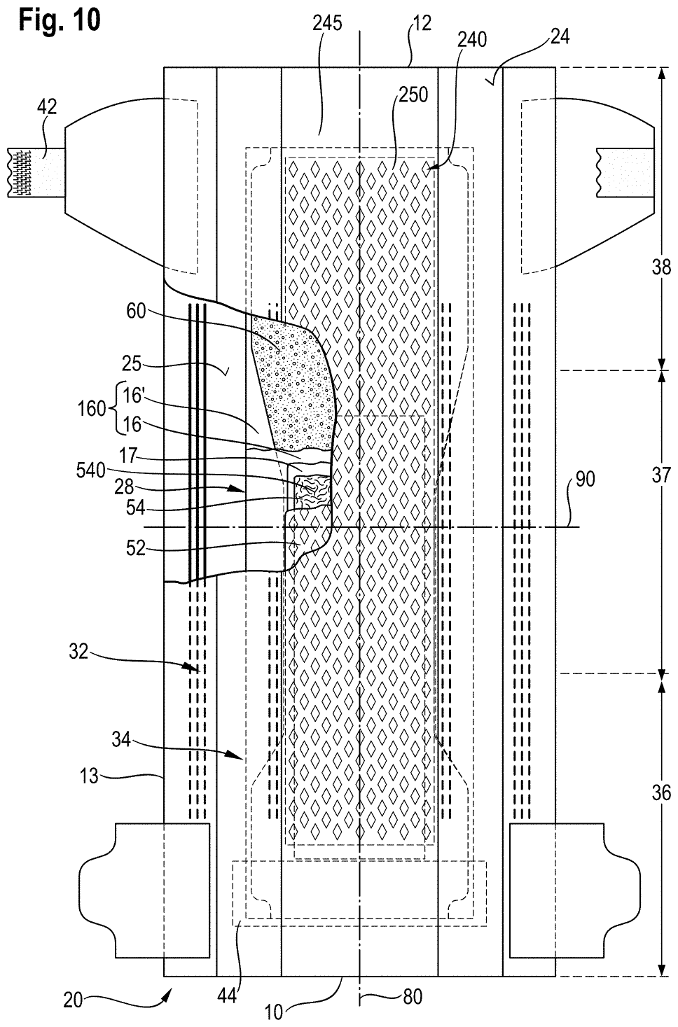

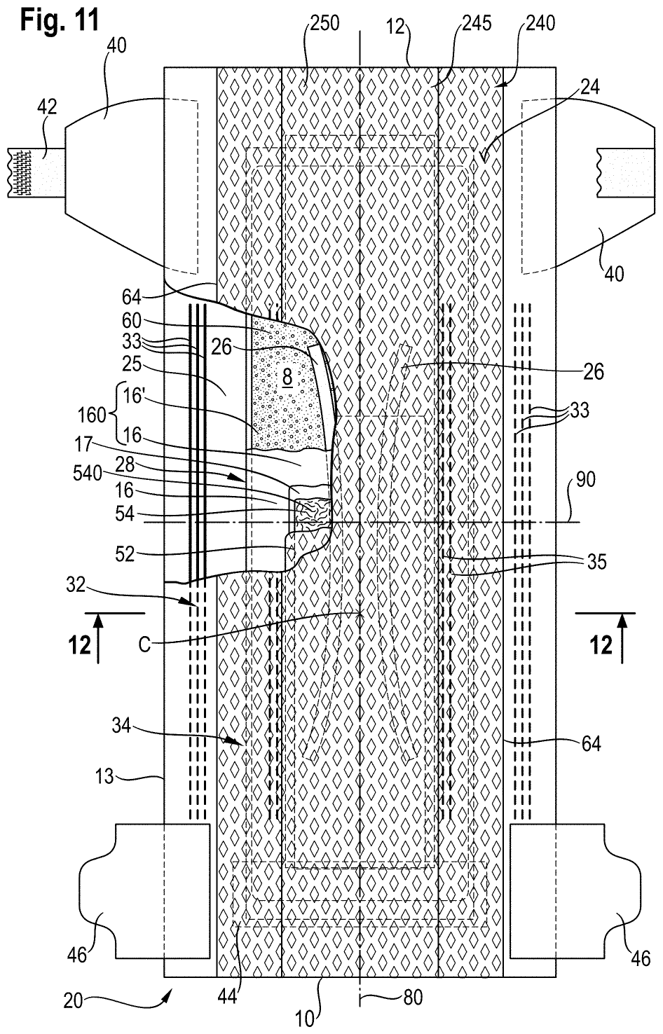

An example absorbent article 20 in which the absorbent core 28 of the invention can be used is a taped diaper 20 as represented in FIG. 1; FIG. 7; FIG. 10 and FIG. 11 with a different absorbent core construction. FIG. 1; FIG. 7; FIG. 10 and FIG. 11 are top plan views of the example diaper 20, in a flat-out state, with portions of the structure being cut-away to more clearly show the construction of the diaper 20. This diaper 20 is shown for illustration purpose only as the invention may be used for making a wide variety of diapers or other absorbent articles.

The absorbent article 20 comprises a three dimensional substrate 240, which may comprise three-dimensional protrusions 250 and form a portion or all of a topsheet 24. Alternatively, the three-dimensional substrate 240 may be a topsheet/acquisition layer laminate 245 formed from a liquid permeable topsheet 24 and an acquisition layer 52 (FIG. 1). In other words, the absorbent article 20 may comprise a liquid permeable topsheet 24 and an acquisition layer 52 characterized in that the topsheet 24 and acquisition layer 52 are joined to form a top sheet/acquisition layer laminate 245.

The absorbent article 20 further comprises a liquid impermeable backsheet 25 and an absorbent core 28 between the topsheet 24 and the backsheet 25. The absorbent article 20 comprises a front edge 10, a back edge 12, and two longitudinal side edges 13. The front edge 10 is the edge of the absorbent article 20 which is intended to be placed towards the front of the user when worn, and the back edge 12 is the opposite edge. The absorbent article 20 may be notionally divided by a longitudinal axis 80 extending from the front edge 10 to the back edge 12 of the absorbent article 20 and dividing the absorbent article 20 in two substantially symmetrical halves relative to this axis, when viewing the absorbent article 20 from the wearer facing side in a flat out configuration, as shown, as an example, in FIG. 1; FIG. 7; FIG. 10 and FIG. 11.

The absorbent article 20 may comprise a distribution layer 54 which comprises a dry-laid fibrous structure. The three-dimensional substrate 240, e.g. a topsheet/acquisition layer laminate 245, is facing towards the body of the wearer when the absorbent article 20 is in use.

The dry-laid fibrous structure may comprise dry-laid fibers 540. The dry-laid fibrous structure may comprise a mixture including superabsorbent polymers and dry-laid fibers. The dry-laid fibers may comprise intra-fiber cross-linked cellulosic fibers.

The distribution layer 54 may for example comprise at least 50% by weight of cross-linked cellulose fibers. The cross-linked cellulosic fibers may be crimped, twisted, or curled, or a combination thereof including crimped, twisted, and curled. This type of material has been used in the past in disposable diapers as part of an acquisition system, for example US 2008/0312622 A1 (Hundorf).

Example chemically cross-linked cellulosic fibers suitable for a distribution layer 54 are disclosed in U.S. Pat. Nos. 5,549,791; 5,137,537; WO95/34329 or US2007/118087. Example cross-linking agents may include polycarboxylic acids such as citric acid and/or polyacrylic acids such as acrylic acid and maleic acid copolymers.

The distribution layer may typically have an average basis weight of from 30 to 400 g/m², in particular from 100 to 300 g/m². The density of the distribution layer may vary depending on the compression of the article, but may be of between 0.03 to 0.15 g/cm³, in particular 0.08 to 0.10 g/cm³ measured at 0.30 psi (2.07 kPa).

The distribution layer 54 may have an average basis weight of from 30 to 400 gsm, in particular from 100 to 300 gsm or from 50 to 250 gsm.

The absorbent article 20 may comprise elasticized gasketing cuffs 32 present between the topsheet 24 and the backsheet 25 and upstanding barrier leg cuffs 34. FIG. 1; FIG. 7; FIG. 10 and FIG. 11 also show other typical diaper components such as a fastening system comprising fastening tabs 42 attached towards the back edge 12 of the absorbent article 20 and cooperating with a landing zone 44 towards the front edge 10 of the absorbent article 20. The absorbent article 20 may also comprise other typical components, which are not represented in the Figures, such as a back elastic waist feature, a front elastic waist feature, transverse barrier cuff(s), a lotion application, etc.

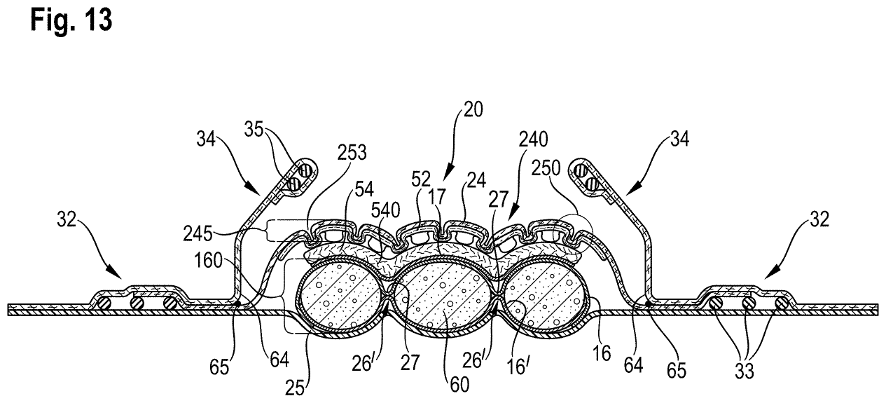

As shown in FIG. 13, the barrier leg cuffs 34 may be delimited by a proximal edge 64 joined to the rest of the article 20, typically the topsheet 24 and/or the backsheet 25, and a free terminal edge intended to contact and form a seal with the wearer's skin. The barrier leg cuffs 34 may be joined at the proximal edge 64 by a bond 65 which may be made for example by adhesive bonding, fusion bonding or combination of known bonding means. Each barrier leg cuff 34 may comprise one, two or more elastic strings 35 to provide a better seal. The gasketing cuffs 32 may be placed transversally outwardly relative to the barrier leg cuffs 34. The gasketing cuffs 32 can provide a better seal around the thighs of the wearer. Usually each gasketing leg cuff 32 will comprise one or more elastic string or elastic element 33 for example between the topsheet 24 and the backsheet 25 in the area of leg openings.

The absorbent article 20 can also be notionally divided by a transversal axis 90 in a front region and a back region of equal length measured on the longitudinal axis, when the absorbent article 20 is in a flat state. The absorbent article's transversal axis 90 is perpendicular to the longitudinal axis 80 and placed at half the length of the absorbent article 20. The length of the absorbent article 20 can be measured along the longitudinal axis 80 from the front edge 10 to the back edge 12 of the absorbent article 20. The topsheet 24, acquisition layer 52, distribution layer 54 and absorbent core 28 each have a width which can be measured from their respective transversal edges and in parallel to the transversal axis 90.

The absorbent article 20 is notionally divided in a front region 36, a back region 38 and a crotch region 37 located between the front and the back region of the absorbent article 20. Each of the front, back and crotch region is ⅓ of the length of the absorbent article 20. The absorbent article may also comprise front ears 46 and back ears 40 as it is known in the art.

The absorbent core 28 of the present invention may comprise as absorbent material 60 a blend of cellulosic fibers (so called “airfelt”) and superabsorbent polymers in particulate form encapsulated in one or more substrates, see for example U.S. Pat. No. 5,151,092 (Buell). Alternatively, the absorbent core 28 may be airfelt free as described in detail below.

Some components of the absorbent article 20 will now be discussed in more details.

### “Airfelt-Free” Absorbent Core 28

The absorbent core 28 of the invention may comprise an absorbent material 60 enclosed within a core wrap 160. The absorbent material 60 may comprise from 80% to 100% of SAP, such as SAP particles, by total weight of the absorbent material 60. The core wrap 160 is not considered as an absorbent material 60 for the purpose of assessing the percentage of SAP in the absorbent core 28.

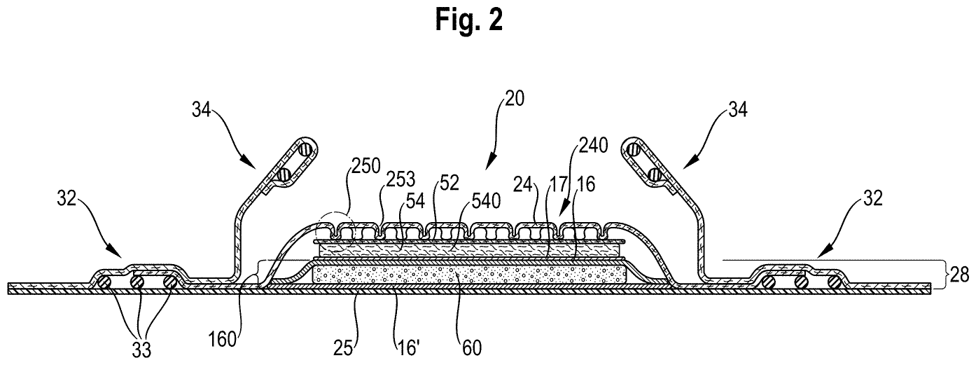

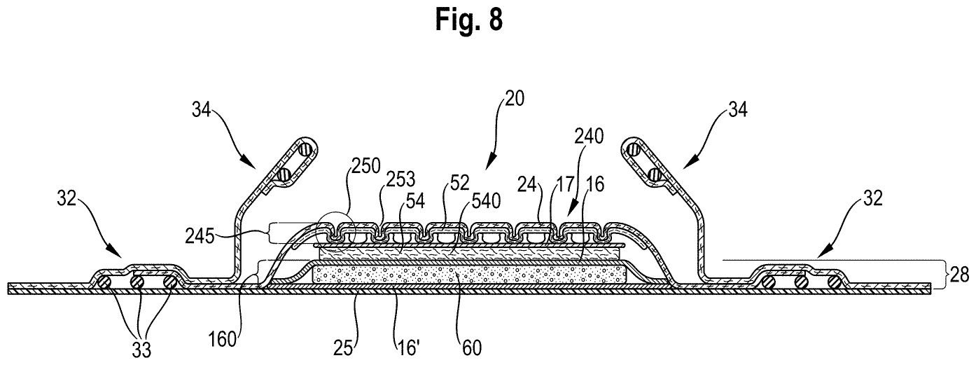

By “absorbent material” it is meant a material which has at least some absorbency and/or liquid retaining properties, such as SAP, cellulosic fibers as well as some hydrophilically treated synthetic fibers. Typically, adhesives used in making absorbent cores have no absorbency properties and are not considered as absorbent material. The SAP content may be substantially higher than 80%, for example at least 85%, at least 90%, at least 95% and even up to and including 100% of the weight of the absorbent material 60 contained within the core wrap 160. This above SAP content substantially higher than 80% SAP may provide a relatively thin absorbent core 28 compared to conventional absorbent cores typically comprising between 40-60% SAP and 40-60% of cellulosic fibers. The absorbent material 60 of the invention may in particular comprise less than 10% weight percent, or less than 5% weight percent, or even be substantially free of natural and/or synthetic fibers. The absorbent material 60 may advantageously comprise little or no cellulosic fibers, in particular the absorbent core 28 may comprise less than 15%, 10%, or 5% (airfelt) cellulosic fibers by weight of the absorbent core 28, or even be substantially free of cellulose fibers. Such absorbent core 28 may be relatively thin and thinner than conventional airfelt cores. FIG. 1, FIG. 2, FIG. 7 and FIG. 8 are illustrations of an absorbent article 20 comprising an “airfelt-free” absorbent core 28.

“Airfelt-free” absorbent cores 28 comprising relatively high amount of SAP with various absorbent core designs have been proposed in the past, see for example in U.S. Pat. No. 5,599,335 (Goldman), EP1447066A1 (Busam), WO95/11652 (Tanzer), US2008/0312622A1 (Hundorf), and WO2012/052172 (Van Malderen).

The absorbent core 28 of the invention may comprise adhesive for example to help immobilizing the SAP within the core wrap 160 and/or to ensure integrity of the core wrap 160 in particular when the core wrap 160 is made of one or more substrates. The core wrap 160 will typically extend over a larger area than strictly needed for containing the absorbent material 60 within.

### Core Wrap

The absorbent material 60 is encapsulated in one or more substrates.

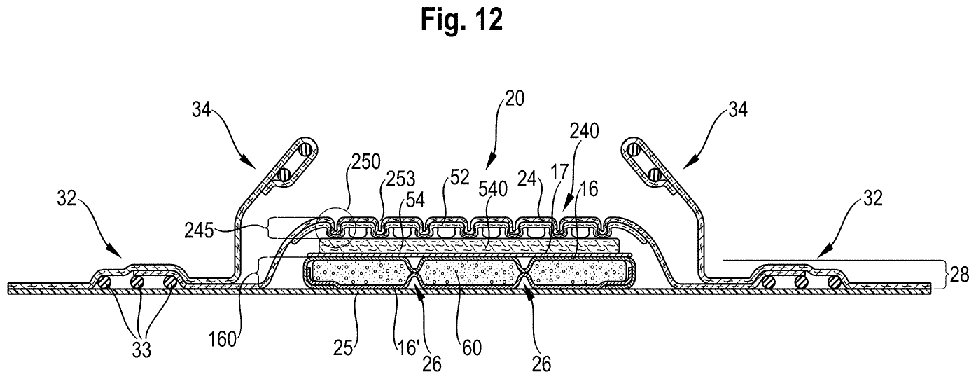

The core wrap 160 comprises a top side 16 facing the topsheet 24 and a bottom side 16′ facing the backsheet 25. The core wrap 160 may be made of a single substrate folded around the absorbent material 60. The core wrap 160 may be made of two substrates (one mainly providing the top side 16 and the other mainly providing the bottom side 16′) which are attached to another, as shown in FIG. 2, as an example. Typical configurations are the so-called C-wrap and/or sandwich wrap. In a C-wrap, as shown in FIG. 12, as an example, the longitudinal and/or transversal edges of one of the substrate are folded over the other substrate to form flaps. These flaps are then bonded to the external surface of the other substrate, typically by bonding with an adhesive. The so called C-wrap construction can provide benefits such as improved resistance to bursting in a wet loaded state compared to a sandwich seal.

The core wrap 160 may be formed by any materials suitable for receiving and containing the absorbent material 60. The core wrap 160 may in particular be formed by a nonwoven web, such as a carded nonwoven, spunbond nonwoven (“S”) or meltblown nonwoven (“M”), and laminates of any of these. For example spunmelt polypropylene nonwovens are suitable, in particular those having a laminate web SMS, or SMMS, or SSMMS, structure, and having a basis weight range of 5 gsm to 15 gsm. Suitable materials are for example disclosed in U.S. Pat. No. 7,744,576, US2011/0268932A1, US2011/0319848A1 or US2011/0250413A1. Nonwoven materials provided from synthetic fibers may be used, such as polyethylene (PE), polyethylene terephthalate (PET) and in particular polypropylene (PP).

### “Airfelt-Free” Absorbent Core 28 Comprising Substantially Absorbent Material Free Areas 26

The absorbent core 28 may comprise an absorbent material deposition area 8 defined by the periphery of the layer formed by the absorbent material 60 within the core wrap 160.

The absorbent core 28 may comprise one or more substantially absorbent material free area(s) 26 which is/are substantially free of absorbent material 60 and through which a portion of the top side 16 of the core wrap 160 is attached by one or more core wrap bond(s) 27 to a portion of the bottom side 16′ of the core wrap 160, as shown in FIGS. 11 and 12. In particular, there can be no absorbent material 60 in these areas. Minimal amount such as contaminations with absorbent material 60 that may occur during the making process are not considered as absorbent material 60. The one or more substantially absorbent material free area(s) 26 may be advantageously confined by the absorbent material 60, which means that the substantially absorbent material free area(s) 26 do(es) not extend to any of the edge of the absorbent material deposition area 8.

If the substantially absorbent material free area 26 extends to any of the edges of the absorbent material deposition area 8, each substantially absorbent material free area 26 may have areas of absorbent material 60 on either side of each substantially absorbent material free area 26.

The absorbent core 28 may comprise at least two substantially absorbent material free areas 26 symmetrically disposed on both sides of the longitudinal axis of the absorbent core 28, as shown in FIG. 11.

The substantially absorbent material free area(s) 26 may be straight and completely oriented longitudinally and parallel to the longitudinal axis but also may be curved or have one or more curved portions.

Furthermore, in order to reduce the risk of liquid bodily exudate leakages, the substantially absorbent material free area(s) 26 advantageously do not extend up to any of the edges of the absorbent material deposition area 8, and are therefore surrounded by and fully encompassed within the absorbent material deposition area 8 of the absorbent core 28. Typically, the smallest distance between a substantially absorbent material free area 26 and the closest edge of the absorbent material deposition area 8 is at least 5 mm “Airfelt free” absorbent cores 28 comprising substantially absorbent material free areas 26 have been proposed, see for example in EP Patent Application No. 12196341.7.

One or more channel(s) 26′ along the substantially absorbent material free area(s) 26 in the absorbent core 28 may start forming when the absorbent material 60 absorbs a liquid and starts swelling. As the absorbent core 28 absorbs more liquid, the depressions within the absorbent core 28 formed by the channel(s) 26′ will become deeper and more apparent to the eye and the touch. The formation of the channel(s) 26′ may also serve to indicate that the absorbent article 20 has been loaded with liquid bodily exudates. The core wrap bond(s) 27 should remain substantially intact at least during a first phase as the absorbent material 60 absorbs a moderate quantity of liquid bodily exudates.

As shown in FIG. 13, when the absorbent material swells, the core wrap bonds 27 remain at least initially attached in the substantially absorbent material free areas 26. The absorbent material 60 swells in the rest of the absorbent core 28 when it absorbs a liquid, so that the core wrap thus forms channels 26′ along the substantially absorbent material free areas 26 comprising the core wrap bonds 27.

### Absorbent Article Having a Topsheet Comprising a Three-Dimensional Substrate

The absorbent article comprises a three-dimensional substrate 240. A portion of, or all of, one or more of three-dimensional substrates may form the topsheet 24.

The three-dimensional substrate 240 may be made from the group consisting of a nonwoven web, a film and combinations thereof.

The three-dimensional substrate 240 may be formed by one or more nonwoven webs. The three-dimensional substrate 240 may be formed by a laminate comprising one or more nonwoven webs and one or more other materials, such as films or cellulosic materials. Combining a nonwoven web and a film will form a laminate.

The three-dimensional substrate 240 may comprise a majority of three-dimensional protrusions 250 having a first Z-directional height. The majority of three-dimensional protrusions 250 protrudes outwardly from the plane P of the three-dimensional substrate 240 forming a base and an opposed distal portion from the plane P. The distal portion of the majority of three-dimensional protrusions 250 extends to a distal end which forms a top peak which is spaced away from the base of the majority of three-dimensional protrusions 250. The base of the majority of three-dimensional protrusions 250 can be defined as the perimeter, which for circular protrusions, is the circumference, where each protrusion of the majority of three-dimensional protrusions 250 starts to protrude outwardly from the plane P of the three-dimensional substrate 240.

The three-dimensional substrate 240 may comprise the majority of three-dimensional protrusions 250 extending outwardly from the plane P of the three-dimensional substrate 240. The majority of three-dimensional protrusions 250 of the three-dimensional substrate 240 may form a three-dimensional surface on a first surface of the three-dimensional substrate 240. The majority of three-dimensional protrusions 250 can be hollow. When viewing from the first surface of the three-dimensional substrate 240, the majority of three-dimensional protrusions 250 protrude from the plane P of the three-dimensional substrate 240. All the protrusions 250 of the three-dimensional substrate 240 may protrude from the plane in the same direction, as shown in FIGS. 2, 3 and 4.

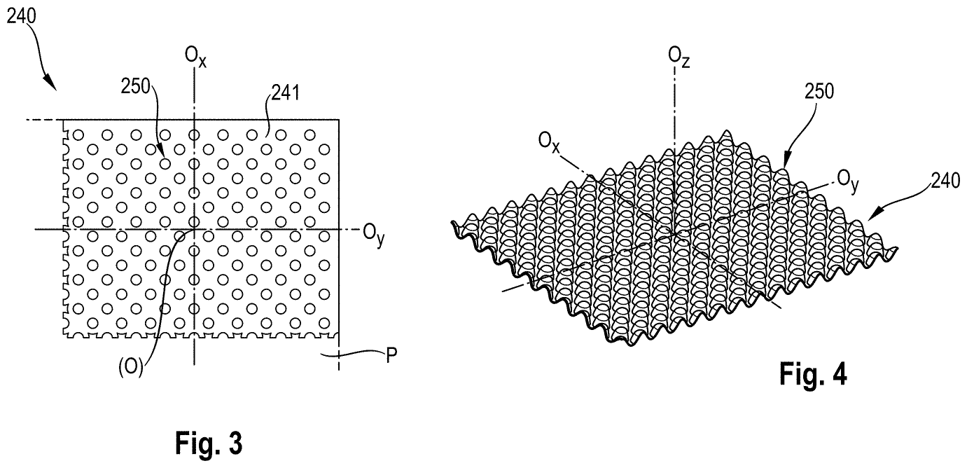

The majority of three-dimensional protrusions 250 may be surrounded by a plurality of land areas 241 of the three-dimensional substrate 240 (see FIG. 3).

In addition to the plurality of land areas 241 and the majority of three-dimensional protrusions 250, the three-dimensional substrate 240 may comprise a plurality of recesses 243 on the first surface of the three-dimensional substrate 10.

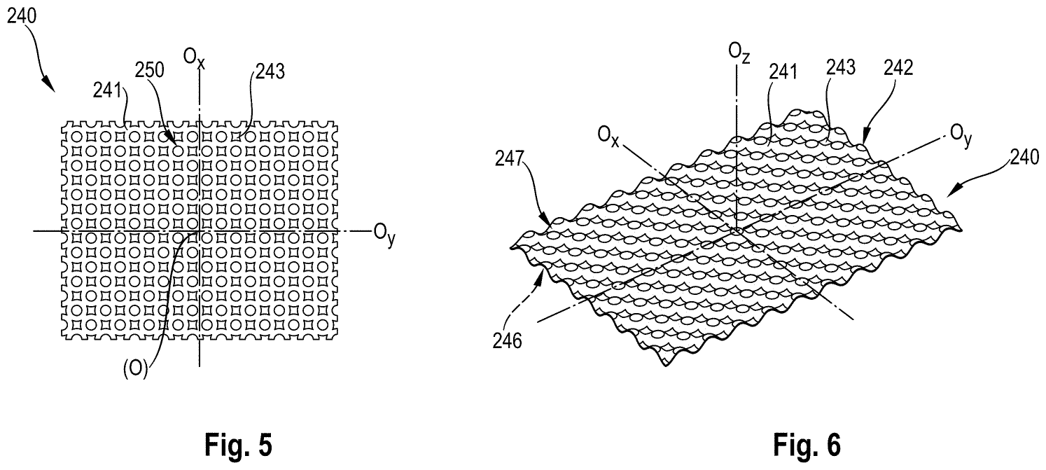

When viewing from the first surface of the three-dimensional substrate 240, the three-dimensional substrate 240 may comprise a plurality of three-dimensional protrusions 250 alternating with a plurality of recesses 243, as shown in FIGS. 5 and 6.

The three-dimensional substrate 240 may comprise a plurality of land areas 241, a plurality of recesses 243, and a plurality of three-dimensional protrusions 250. The plurality of land areas 241, the plurality of recesses 243, and the plurality of three-dimensional protrusions 250 may together form a three-dimensional surface on the first surface 247 of the three-dimensional substrate 240.

As shown in FIGS. 5 and 6, as an example, the plane P of the three-dimensional substrate 240 may comprise a continuous land area.

The three-dimensional substrate 240 may have the following repetitive grid pattern when viewing the three-dimensional substrate 240 from the first surface 247 of the three-dimensional substrate: Each three-dimensional protrusion 250 of the three-dimensional substrate 240 may be positioned at a center of a square wherein each corner of the square includes a further three-dimensional protrusion 250.

If the three-dimensional substrate 240 also comprises a plurality of recesses 243, each recess 243 may be positioned substantially at the center of each edge of the square. The plurality of land areas 241 may then encompass the space between the plurality of three-dimensional protrusions 250 and the plurality of recesses 243.

The majority of the three-dimensional protrusions 250 may be generally dome-shaped when viewing from the first surface 247 of the three-dimensional substrate 240 and may be hollow arch-shaped when viewing from the opposite second surface 246 of the three-dimensional substrate 10.

The majority of the three-dimensional protrusions 250 may alternate with the recesses 243 in a direction generally perpendicular with the longitudinal axis of the three-dimensional substrate 10. The majority of the three-dimensional protrusions 250 may also alternate with the recesses 13 in a direction generally parallel with a longitudinal axis of the three-dimensional substrate 10.

Two or more adjacent three-dimensional protrusions 250 may be separated from each other by a recess 243 and one or more land areas 241 in a direction generally perpendicular to the longitudinal axis or in a direction generally parallel to the longitudinal axis of the three-dimensional substrate 240.

Two or more adjacent recesses 243 may be separated by a three-dimensional protrusion 250 and one or more land areas 241 in a direction generally perpendicular to the longitudinal axis or in a direction generally parallel to the longitudinal axis of the three-dimensional substrate 240.

The land areas 241 may fully surround the recesses 243 and the three-dimensional protrusions 250. The land areas 241 may together form a generally continuous grid through the three-dimensional substrate 240, while the three-dimensional protrusions 250 and the recesses 243 may be discrete elements throughout the three-dimensional substrate 240 according to the repetitive grid pattern as defined above.

Each recess 243 of the plurality of recesses may comprise an aperture. Advantageously, the aperture may be located at the base of the recess 243.

The three-dimensional substrate 240 of the absorbent article 20 may comprise a plurality of recesses 243 and a plurality of land areas 241. The land areas 241 surround at least a majority of the plurality of three-dimensional protrusions 250 and a plurality of the recesses 243.

The plurality of recesses 243, the plurality of three-dimensional protrusions 250, and the plurality of land areas 241, together form a first three-dimensional surface on a first side of the three-dimensional substrate 240 and a second three-dimensional surface on a second side of the three-dimensional substrate 240.

A majority of the recesses 243 may preferably define an aperture at a location most distal from a top peak of an adjacent three-dimensional protrusion 250. The majority of the recesses may have a Z-directional height in the range of 500 μm to 2000 μm according to the Recess Height Test Method.

The three-dimensional substrate 240 may have an overall Z-directional height in the range of 1000 μm to 6000 μm according to the Overall Substrate Height Test Method.

The majority of the three-dimensional protrusions 250 extending outwardly from the plane of the three-dimensional substrate 240 may represent at least 20% or at least 30% or at least 40% or more than 50%, or more than 70%, or more than 90%, or more than 95% of the total area of the three-dimensional substrate 240.

The majority of the three-dimensional protrusions 250 extending outwardly from the plane of the three-dimensional substrate 240 may represent no more than 70% or no more than 60% or no more than 50% of the total area of the three-dimensional substrate 240.

The majority of the three-dimensional protrusions 250 of the three-dimensional substrate 240 may have a Z-directional height from 500 μm to 4000 μm or from 300 μm to 3000 μm or from 500 μm to 3000 μm or from 800 μm to 1400 μm or from 1100 μm to 1200 μm. The Z-directional height of the majority of the three-dimensional protrusions 250 of the three-dimensional substrate 240 is measured according to the Projection Height Test Method described herein.

The majority of the recesses 243 of the three-dimensional substrate 240 may have a Z-directional height from 100 μm to 3000 μm or from 300 μm to 2000 μm or from 500 μm to 1500 μm or from 700 μm to 1000 μm. The Z-directional height of the recesses 243 of the three-dimensional substrate 240 is measured according to the Recess Height Test Method described herein.

The three-dimensional substrate 240, or portions thereof, may have an overall Z-directional height from 700 μm to 6000 μm or from 750 μm to 4000 μm or from 1000 μm to 2500 μm or from 1750 μm to 2300 μm anywhere in the spool 1. The overall Z-directional height of the three-dimensional substrate 240, or portions thereof, is measured according to the Overall Substrate Height Test Method described herein.

The three-dimensional substrates of the present invention may comprise one or more colors, dyes, inks, indicias, patterns, embossments, and/or graphics. The colors, dyes, inks, indicias, patterns, and/or graphics may aid the aesthetic appearance of the three-dimensional substrates.

### Absorbent Article Having a Topsheet/Acquisition Layer Laminate as the Three-Dimensional Substrate

The absorbent article 20 comprises a three-dimensional substrate 240 which may be a topsheet/acquisition layer laminate 245 comprising three-dimensional protrusions extending from a plane of the laminate, as shown in FIGS. 7-13. The three-dimensional substrate 245 may comprise a topsheet 24 comprising a plurality of fibers and an acquisition layer 52 comprising a plurality of fibers characterized in that the topsheet 24 and the acquisition layer 52 are joined to form a topsheet/acquisition layer laminate 245.

The topsheet 24 has a first and second surface and the acquisition layer 52 has a first and second surface. The first surface of the topsheet will be facing towards the body of the wearer when the absorbent article 20 is in use.

The topsheet 24 and acquisition layer 52 are aligned in a face to face relationship such that the second surface of the topsheet 24 is in contact with the first surface of the acquisition layer 52. The topsheet 24 and the acquisition layer 52 can be simultaneously mechanically deformed and combined together to provide a topsheet/acquisition layer laminate 245 having three-dimensional protrusions 250. This means that both topsheet 24 and acquisition layer 52 can be mechanically deformed and combined together at the same time. The three-dimensional protrusions 250 extend from a plane of the topsheet/acquisition layer laminate 245.

The three-dimensional protrusions 250 are formed from the fibers of the topsheet 24 and the acquisition layer 52. A majority of the three-dimensional protrusions 250 comprise a base 256 forming an opening and having a protrusion base width, an opposed distal portion 257, and one or more side walls 255 between the base 256 and the distal portion 257 of the three-dimensional protrusion. The base 256, distal portion 257 and the one or more side walls 255 are formed by fibers such that the majority of the three-dimensional protrusions 250 has only one opening at the base, as shown in FIG. 17A, as an example. At least 50% or at least 80% of the three-dimensional protrusions 250 of the topsheet/acquisition layer laminate 245 may only have openings at the base 256. The majority of the three-dimensional protrusions 250 can be obtained by one of the two mechanical processes described in detail below.

The majority of the three-dimensional protrusions 250 may be more than 50% or more than 60% or more than 70% or more than 80% or more than 90% or more than 95% or more than 98% of the three-dimensional protrusions 250 in the topsheet/acquisition layer laminate 245.

The fibers may substantially or completely surround the one or more side walls 255 of the majority of the three-dimensional protrusions 250. This means that there are multiple fibers which contribute to form a portion of the side walls 255 and distal portion 257 of a three-dimensional protrusion 250. The term “substantially surround” does not require that each individual fiber be wrapped substantially or completely around the side walls 255 of the three-dimensional protrusions 250.

The topsheet/acquisition layer laminate 245 has a first surface comprising the second surface of the acquisition layer 52.

A portion of the backsheet 25 can be joined to a portion of the topsheet 24 of the topsheet/acquisition layer laminate 245 such that the first surface of the topsheet/acquisition layer laminate 245 is facing towards the backsheet 25.

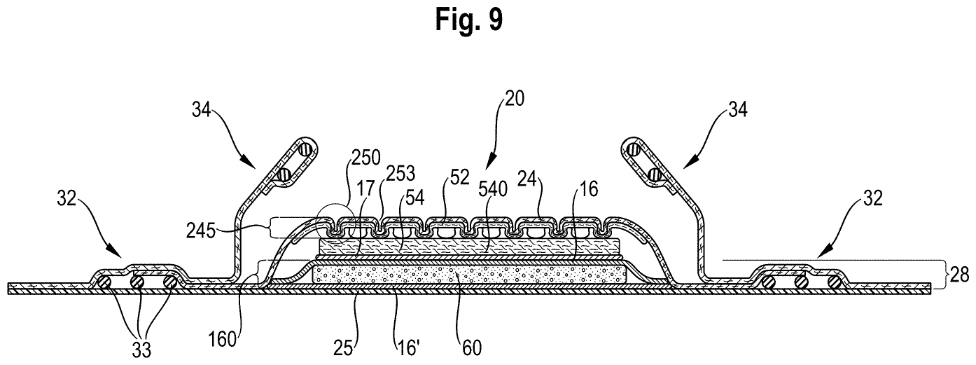

The absorbent article 20 may comprise gasketing cuffs 32. The majority of the three-dimensional protrusions 250 of the topsheet/acquisition layer laminate 245 may at least be present in the acquisition layer 52 and in the topsheet 24, in the area where the topsheet 24 overlaps the acquisition layer 52 in the topsheet/acquisition layer laminate 245. However, the majority of the three-dimensional protrusions 250 of the topsheet/acquisition layer laminate 245 may be present in the area which extends parallel to the transversal axis 90 of the absorbent article 20. The majority of the three-dimensional protrusions 250 of the topsheet/acquisition layer laminate 245 may be present in the area which extends parallel to the longitudinal axis 80 of the absorbent article 20, but which does not extend beyond the area where gasketing cuffs 32 is attached to the absorbent article 20, in particular to the topsheet 24, as shown in FIG. 8 or 9. In that case, the majority of the three-dimensional protrusions 250 which are formed in the topsheet 24 of the topsheet/acquisition layer laminate 245, are formed from the fibers of the topsheet 24.

Alternatively, the majority of the three-dimensional protrusions of the topsheet/acquisition layer laminate 245 may be present in the acquisition layer and in the topsheet in the area which extends parallel to the transversal axis 90 of the absorbent article 20 such that the area comprising the three-dimensional protrusions of the topsheet 24 overlaps the acquisition layer 52. The length of the area of majority of the three-dimensional protrusions of the topsheet/acquisition layer laminate 245 may be from 5% to 60% or from 10% to 40% wider than the length of the acquisition layer 52 of the topsheet/acquisition layer laminate 245. The majority of the three-dimensional protrusions 250 of the topsheet/acquisition layer 245 may be present in the area which extends parallel to the longitudinal axis 80 of the absorbent article 20 such that the area comprising the three-dimensional protrusions of the topsheet 24 overlaps the acquisition layer 52. The width of the area of the majority of the three-dimensional protrusions of the topsheet/acquisition layer 245 may be from 5% to 60% or from 10% to 40% wider than the width of the acquisition layer 52 of the topsheet/acquisition layer laminate 245. In that case, the three-dimensional protrusions 250 which are formed in the topsheet 24 of the top sheet/acquisition layer laminate 245, are formed from the fibers of the topsheet 24.

In still another alternative, the majority of the three-dimensional protrusions 250 of the topsheet/acquisition layer laminate 245 may only be present where the topsheet 24 overlaps the acquisition layer 52 in the topsheet/acquisition layer laminate 245, as shown in FIG. 10.

Hence, the majority of the three-dimensional protrusions 250 can provide an impression of depth and can support the caregiver's perception that the absorbent article 20 is well able to absorb the liquid bodily exudates.

The majority of the three-dimensional protrusions 250 of the topsheet/acquisition layer laminate 245 may have a measured protrusion height from 0.3 mm to 5 mm or from 0.7 mm to 3 mm or from 1.0 mm to 2.0 mm according to the Protrusion Height Test Method as described below.

The majority of the three-dimensional protrusions 250 of the topsheet/acquisition layer laminate 245 may have a measured protrusion base width of the three-dimensional protrusions 250 from 0.5 mm to 10 mm or from 0.5 to 5 mm or from 0.5 mm to 3.0 mm or from 1.0 mm to 2.5 mm or from 1.5 mm to 2.5 mm according to the Protrusion Base Width Test Method as described below. The majority of the three-dimensional protrusions 250 having a shape with a specific protrusion height and protrusion base width can contribute to provide an impression of depth and can support the caregiver's perception that the absorbent article 20 is well able to absorb the liquid bodily exudates.

The majority of the three-dimensional protrusions 250 provides void volume to receive the liquid bodily exudates. At the same time, the topsheet/acquisition layer laminate 245 is in close contact with the underlaying layer, i.e. the distribution layer 54. The distribution layer 54 made of unconsolidated dry-laid fibers 540 of a dry-laid fibrous structure or a wet-laid fibrous structure may sink in the depressions provided by the majority of the three-dimensional protrusions 250 of the topsheet/acquisition layer laminate 245 (not shown in the schematic Figures). The distribution layer 54 may follow the shape of the majority of the three-dimensional protrusions. Hence, the liquid bodily exudates are transmitted more efficiently from the topsheet/acquisition layer laminate 245 to the distribution layer 54, which can improve the dryness of the topsheet 24 of the topsheet/acquisition layer laminate 245. Rewet can be reduced at the skin of the wearer. The topsheet/acquisition layer laminate 245 may also enable more efficient use of an absorbent core 28. Overall, the topsheet 24 of the topsheet/acquisition layer laminate 245 can have an improved dryness than a three-dimensional topsheet 24 placed on top of an acquisition layer 52.

The majority of the three-dimensional protrusions 250 may comprise void areas 253 which do not contact the skin of the wearer. The absorbent article 20 may be in less contact with the skin of the wearer in comparison with a flat topsheet. The void areas 253 of the topsheet/acquisition layer laminate 245 can help the air to permeate between the skin of the wearer and the topsheet/acquisition layer laminate 245. The void areas 253 of the topsheet/acquisition layer laminate 245 can improve the breathability of the topsheet/acquisition layer laminate 245.

In addition to improve dryness, the void areas 253 of the topsheet/acquisition layer laminate 245 can also allow feces to be absorbed and acquired within them. In that case, the present invention is suitable to absorb feces of relatively low viscosity.

A width of the acquisition layer 52 in a direction parallel to the transversal axis 90 may be less than a width of the topsheet 24 in a direction parallel to the transversal axis 90. If the width of both topsheet 24 and acquisition layer 52 were the same, wicking of the liquid bodily exudates underneath the gasketing cuffs 32 might occur. Hence, the liquid bodily exudates might not be properly absorbed by the absorbent core 28, which may lead to leakage of the liquid bodily exudates out of the absorbent article 20. If the width of the acquisition layer 52 in a direction parallel to the transversal axis 90 is less that the width of the topsheet 24 in a direction parallel to the transversal axis 90, the acquisition layer 52 which may receive the liquid bodily exudates from the topsheet 24 can directly transmit the liquid bodily exudates to the distribution layer 54 in order to be subsequently absorb by the absorbent core 28. Hence, the liquid bodily exudates temporary stored in the acquisition layer 52 of the topsheet/acquisition layer laminate 245 will not readily be drawn towards and underneath the gasketing cuffs 32 by capillary forces. Leakage can thus be reduced by having the width of the acquisition layer 52 less that the width of the topsheet 24 in the topsheet/acquisition layer laminate 245 in a direction parallel to the transversal axis 90.

In order to help reducing leakage and rewet, the width of the acquisition layer 52 of the topsheet/acquisition layer laminate 245 in a direction parallel to the transversal axis 90 may not be more than 40% wider than the width of the distribution layer 54 and/or more than 20% wider than the width of the absorbent core 28 in a direction parallel to the transversal axis 90. In that case, the liquid bodily exudates may not accumulate at or adjacent to the transversal edges of the acquisition layer. Wicking of the liquid bodily exudates underneath the gasketing cuffs 32 is prevented. Indeed, when the acquisition layer 52 of the tospheet/acquisition layer laminate 245 is no more than 20% wider than the width of the absorbent core 28, the liquid bodily exudates can readily be transported into the absorbent core 28, which can efficiently drain the fluid from the acquisition layer 52 into the absorbent core 28. Wicking of the liquid bodily exudates form the acquisition layer 52 underneath the gasketing cuffs 32 is prevented.

The acquisition layer 52 can receive the liquid bodily exudates that pass through the topsheet 24 and can distribute them to underlying absorbent layers. In such a case, the topsheet 24 in the topsheet/acquisition layer laminate 245 may be less hydrophilic than the acquisition layer 52. The topsheet 24 of the topsheet/acquisition layer laminate 245 can be readily dewatered.

In order to enhance dewatering of the topsheet 24 of the topsheet/acquisition layer laminate 245, the pore size of the acquisition layer 52 may be reduced. For this, the acquisition layer 52 may made of fibers with relatively small denier. The acquisition layer 52 may also have an increased density. The acquisition layer 52 may also have an increased hydrophilicity.

A length of the acquisition layer 52 in the topsheet/acquisition layer laminate 245 in a direction parallel to the longitudinal axis 80 may be less than a length of the topsheet 24 taken along the longitudinal axis 80 of the absorbent article 20, as shown in FIG. 10. When the length of the acquisition layer 52 in the topsheet/acquisition layer laminate 245 is less than the length of the topsheet 24, the liquid bodily exudates cannot be readily drawn towards the longitudinal edges (10, 12) of the absorbent article 20, which reduces leakage.

The length of the acquisition layer 52 in the topsheet/acquisition layer laminate 245 may be less than the length of the absorbent core 28 taken along the longitudinal axis 80 of the absorbent article 20, see for example FIG. 10.

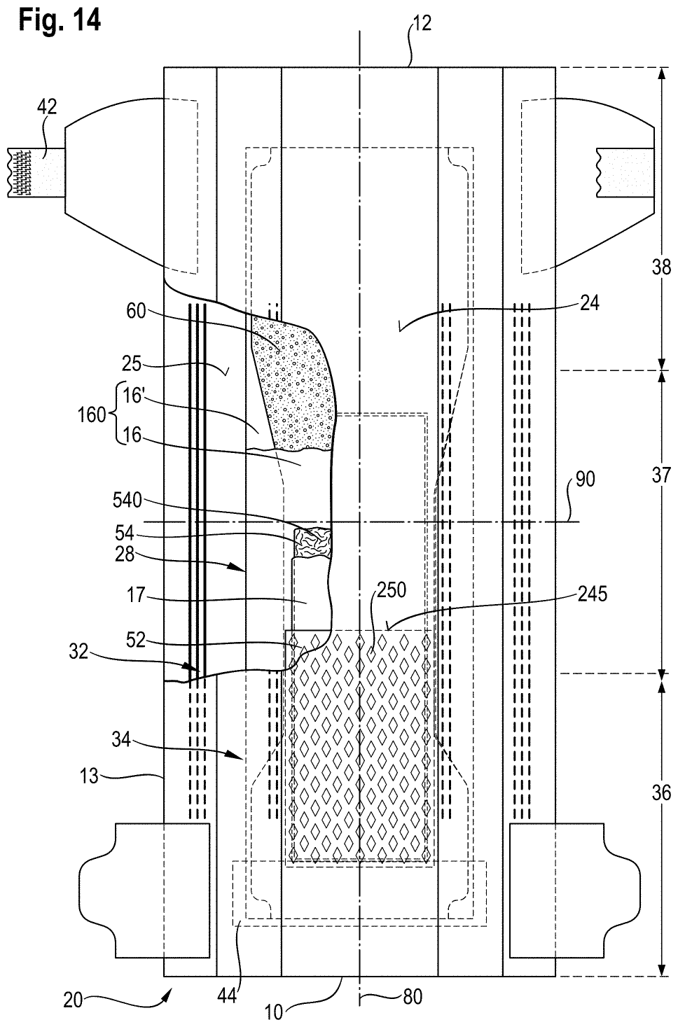

The acquisition layer 52 of the topsheet/acquisition layer laminate 245 may be positioned in the front region 36 and at least partially in the crotch region 37 of the absorbent article 20, as shown in FIG. 14. In that case, positioning the acquisition layer 52 of the topsheet/acquisition layer laminate 245 in the front region 36 of the absorbent article 20 helps for acquiring and distributing the liquid bodily exudates such as urine, around the pee point of the wearer.

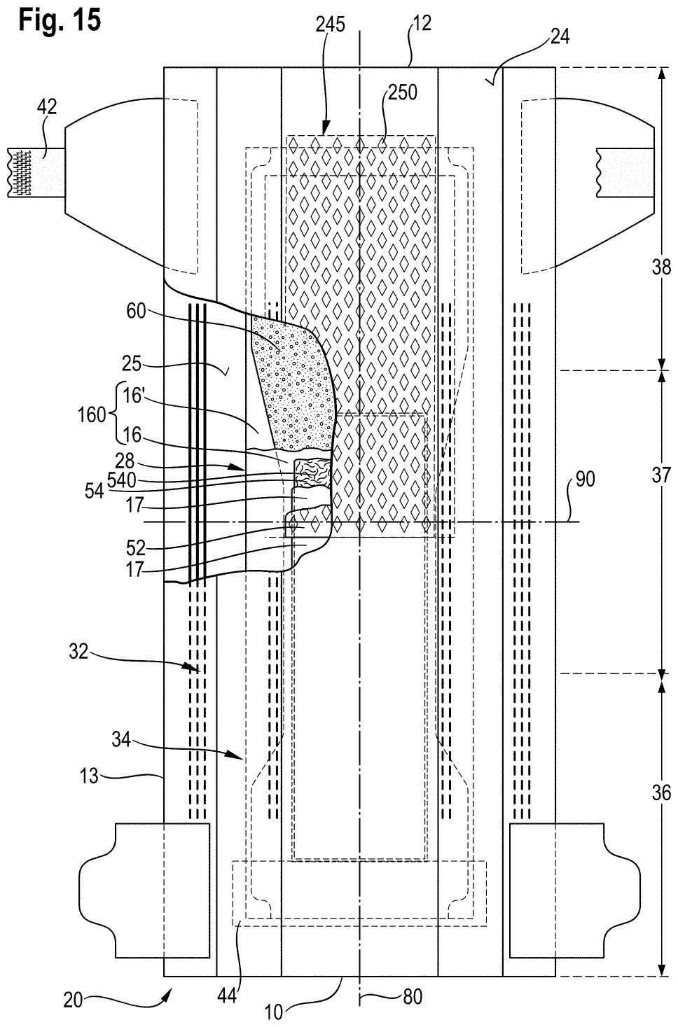

The acquisition layer 52 of the topsheet/acquisition layer laminate 245 may be positioned in the back region 38 and at least partially in the crotch region 37 of the absorbent article 20, as shown in FIG. 15. Positioning the acquisition layer 52 of the topsheet/acquisition layer laminate 245 in the back region 38 of the absorbent article 20 helps at acquiring the feces of the wearer, especially when the feces have a low viscosity.

The three-dimensional protrusions 250 of the topsheet/acquisition layer laminate 245 may protrude towards the backsheet 25 or towards the body of the wearer when the absorbent article is in use.

The topsheet/acquisition layer laminate 245 may be notionally divided into a first and second area. The first area may comprise three-dimensional protrusions 250 which protrude towards the backsheet 25. The second area may comprise three-dimensional protrusions 250 which protrude towards the body of the wearer when the absorbent article is in use.

For instance, the first area may be located in the front region 36 and at least partially in the crotch region 37 of the absorbent article 20.

Having the first area where the three-dimensional protrusions 250 of the topsheet/acquisition layer laminate 245 protrude towards the backsheet 25 can help acquiring and absorbing the liquid bodily exudates to the absorbent core 28. Having the second area where the three-dimensional protrusions 250 of the topsheet/acquisition layer laminate 245 protrude towards the body of the wearer when the absorbent article is in use can improve cleaning the body from the exudates. Hence, a combination of the first and second area can allow the absorbent article to better perform.

The three-dimensional protrusions 250 may be disposed in any suitable arrangement across the plane of the topsheet/acquisition layer laminate 245. Suitable arrangements include, but are not limited to: staggered arrangements, and zones. In some cases, the topsheet/acquisition layer laminate 245 may comprise both three-dimensional protrusions 250 and other features known in the art such as embossments and apertures. The three-dimensional protrusions 250 and other features may be in separate zones, be intermixed, or overlap.

### The Mechanical Deformations and the Resulted Three-Dimensional Protrusions

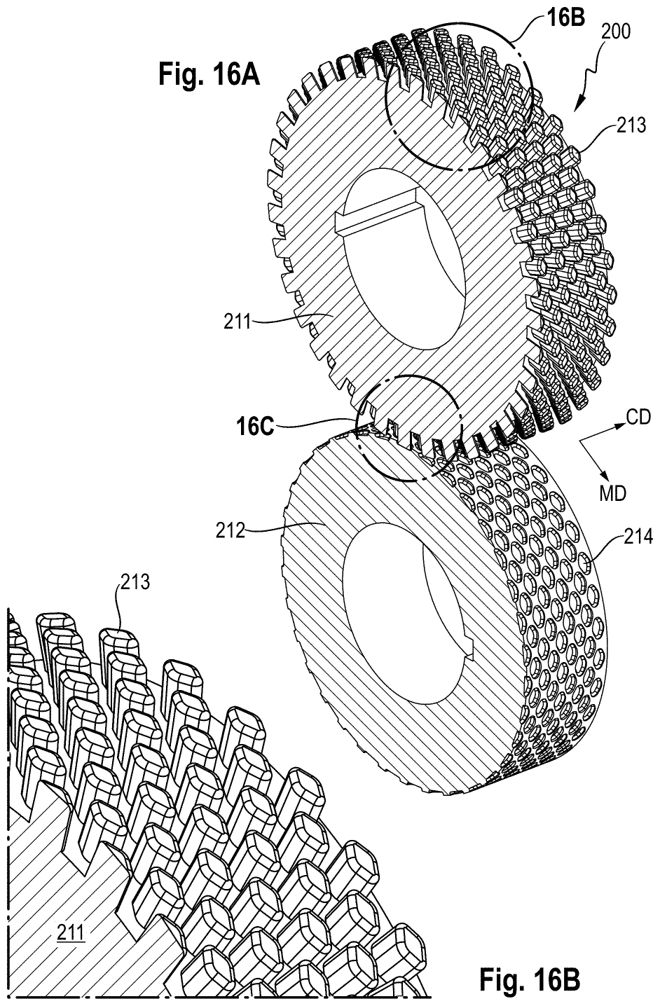

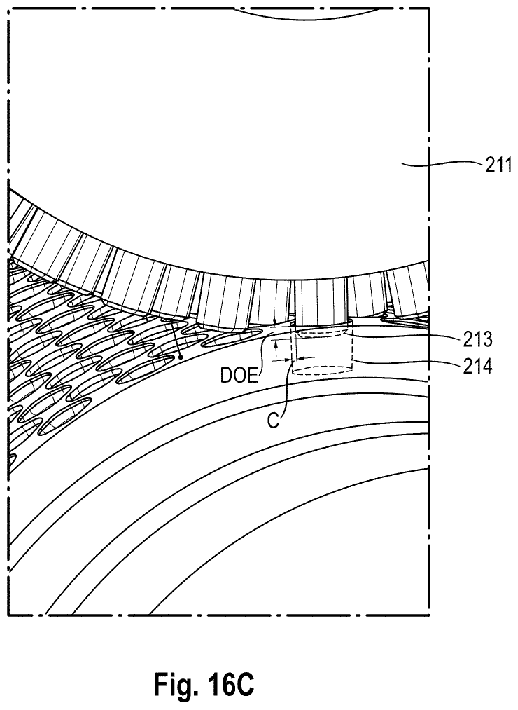

The topsheet/acquisition layer laminate 245 may be obtainable by the process comprising the following step of providing a first and second forming member (211, 212) having a machine direction and a cross direction orientation, as shown in FIGS. 16A, 16B and 16C. The first and second forming member (211, 212) may be drum-shaped, generally cylindrical or plate-shaped.

The first forming member 211 of the apparatus 200 may have a surface comprising a plurality of discrete, spaced apart male forming elements 213 having a base that is joined to the first forming member 211, a top that is spaced away from the base, and sides that extend between the base and the top of the male forming elements 213. The male forming elements 213 may have a plan view periphery, and a height.

The top on the male forming elements 213 may have a rounded diamond shape, see for example FIG. 16A, with vertical sidewalls and a radiused or rounded edge at the transition between the top and the sidewalls of the male forming element 213.

The second forming member 212 may have a surface comprising a plurality of recesses 214 in the second forming member 212. The recesses 214 may be aligned and configured to receive the respective male forming elements 213 therein. Hence, each recess 214 of the second forming member 212 may be sufficiently large to be able to receive each respective male forming element 213 of the first forming member 211. The recesses 214 may have a similar shape as the male forming elements 213. The depth of the recesses 214 may be greater than the height of the male forming elements 213.

The first and second forming member (211, 212) may be further defined by a depth of engagement (DOE) which is a measure of the level of intermeshing of the first and second forming member (211, 212), as shown in FIG. 16C. The depth of engagement (DOE) may be measured from the tip of the male forming elements 213 to the outermost portion of the surface of the second forming member 212 which portions are not within a recess 214. The depth of engagement (DOE) may range from 1.5 mm to 5.0 mm or from 2.5 mm to 5.0 mm or from 3.0 mm to 4.0 mm.

The first and second forming member (211, 212) may be defined by a clearance (C) between the first and second forming member (211, 212) as shown in FIG. 16C. The clearance (C) is the distance between the side wall of the male forming element 213 and the side wall of the recess 214. The clearance (C) may range from 0.1 mm to 2 mm or from 0.1 mm to 1.5 mm from 0.1 mm to 1 mm.

The topsheet 24 and the acquisition layer 52 may be engaged together between the first and second forming members (211, 212) and be mechanically deformed and combined together to form the topsheet/acquisition layer laminate 245. The topsheet/acquisition layer laminate 245 comprises thus deformations forming three-dimensional protrusions 250. The three-dimensional protrusions 250 may have a ratio of length to width. The ratio of length to width can range from 10:1 to 1:10.

The topsheet/acquisition layer laminate 245 may be notionally divided into a first and second area. The first and/or second area of the topsheet/acquisition layer laminate 245 may comprise three-dimensional protrusions 250 having different shapes.

Viewed from a cross-sectional view, i.e. in a Z-direction, the three-dimensional protrusions 250 may have any suitable shapes which include, but are not limited to: bulbous-shaped, conical-shaped and mushroom shaped.

Viewed from above, the three-dimensional protrusions 250 may have any suitable shapes which include, but are not limited to: circular, diamond-shaped, round diamond-shaped, U.S. football-shaped, oval-shaped, clover-shaped, triangular-shaped, tear-drop shaped and elliptical-shaped protrusions. The three-dimensional protrusions 250 may be non-circular.

The three-dimensional protrusions 250 may form, in conjunction, one or more graphics. Having graphics can support the caregiver's perception that the absorbent article is well able to absorb the liquid bodily exudates.

FIG. 17A-FIG. 17F shows different alternatives of three-dimensional protrusions 250. A bulbous-shaped protrusion may be one type of three-dimensional protrusions 250 which may be obtained by the process step described above using the apparatus 200. The topsheet/acquisition layer laminate 245 may comprise a plurality of three-dimensional protrusions 250 protruding towards the backsheet 25.

The three-dimensional protrusion 250 is formed from the fibers of the topsheet 24 and the acquisition layer 54. The three-dimensional protrusion 250 is defined by a base 256 forming an opening, an opposed enlarged distal portion 257 that extends to a distal end 259 and one or more side walls 255 between the base 256 and the distal portion 257. The base 256, distal portion 257 and the one or more side walls 255 are formed by fibers such that the three-dimensional protrusion 250 has only one opening at the base 256. At least 50% or at least 80% of the three-dimensional protrusions 250 of the topsheet/acquisition layer laminate 245 may only have openings at the base 256. The side wall 255 may be substantially continuous. For instance, the side wall 255 may be spherical or conical. The three-dimensional protrusion 250 may comprise more than one side wall 255, e.g. in a pyramidal-shaped protrusion. The fibers may substantially or completely surround the one or more side walls 255 of the three-dimensional protrusions 250.

The topsheet 24 and the acquisition layer 52 may be both extensible. The fibers composing the topsheet 24 and acquisition layer 52 may elongate and/or may be mobile, such that the topsheet 24 and acquisition layer 52 are able to stretch to be nested together.

Generally, the extensibility of the materials composing the topsheet 24 and acquisition layer 52 can be selected according to the desired sizes of the three-dimensional protrusions 250. If relatively large three-dimensional protrusions 250 are desired, materials with a relatively higher extensibility will be chosen.

The topsheet 24 or acquisition layer 52 may be capable of undergoing an apparent elongation of equal to or greater than at least 100% or 110% or 120% or 130% up to 200% along longitudinal and/or transversal axis of the absorbent article at or before reaching the breaking force according to the Test Method as set out above. It might be desired to have three-dimensional protrusions 250 which are larger either along the longitudinal or transversal axis of the absorbent article. For this, the materials composing the topsheet 24 and acquisition layer 52 can be thus more extensible in either along the longitudinal versus transversal axis of the absorbent article or vice versa.

The three-dimensional protrusion 250 may comprise a void area 253 which is the portion of the three-dimensional protrusion 251A which does not comprise any fibers or very little fibers. The three-dimensional protrusion 250 may be defined by a protrusion base width WB₁ of the base 256 forming an opening which is measured from two side walls of the inner portion 251A at the base 256. The three-dimensional protrusion 250 may be defined by a width WD₂ of the void area 253 which is the maximum interior width measured between two side walls of the inner three-dimensional protrusion 251A or which is the maximum diameter of the side wall when the distal portion has a substantially circular shape. The maximum interior width WD₂ of the void area 253 at the distal portion may be greater than the protrusion base width WB₁ of the base 256 of the three-dimensional protrusion 250. This is the case for some types of three-dimensional shapes, such as bulbous shapes as exemplified in FIG. 17B but nor for conical shape. The protrusion base width WB₁ of the base 256 of the three-dimensional protrusion 250 may range from 1.5 mm to 15 mm or from 1.5 mm to 10 mm or from 1.5 mm to 5 mm or from 1.5 mm to 3 mm Measurements of the dimensions of the protrusion base width WB₁ of the base 256 and the width WD₂ of the distal portion 257 can be made on a photomicrograph.

When the size of the protrusion base width WB₁ of the base 256 is specified herein, it will be appreciated that if the openings are not of uniform width in a particular direction, the protrusion base width, WB₁, is measured at the widest portion. Measurements of the protrusion base width WB₁ of the base 256 or the maximum interior width WD₂ of the void area 253 at the distal portion 257 can be made on a photomicrograph at 20× magnification.

As the plurality of fiber (254A, 254B) composing the three-dimensional protrusions 2561 may be present in the one or more side walls 255 of the three-dimensional protrusions 250, the three-dimensional protrusions may not collapse on one side and close off the opening at the base 256 when compressive forces are applied on the topsheet/acquisition layer laminate 245. The opening at the base 256 may be maintained and may create a ring of increased opacity around the opening at the base 256 when the three-dimensional protrusion has been compressed. Hence, the three-dimensional protrusion 250 can be preserved and remain visible to the consumer when viewing the absorbent article 20 from the topsheet 24. The three-dimensional protrusion 250 can be preserved after being subjected to any inherent compressive forces due to the process or the step of compressing the absorbent articles comprising the topsheet/acquisition layer laminate 245 prior to be filled in a packaging.

In other words, the three-dimensional protrusions 250 may have a degree of dimensional stability in the X-Y plane when a Z-direction force is applied to the three-dimensional protrusions 250. It is not necessary that the collapsed configuration of the three-dimensional protrusions 250 be symmetrical, only that the collapsed configuration prevent the three-dimensional protrusions 250 from flopping over or pushing back into the original plane of the topsheet/acquisition layer laminate 245. Without wishing to be bound to any particular theory, the wide base 256 and large cap 52 (greater than the protrusion base width of the base opening 256), combined with the lack of a pivot point, causes the three-dimensional protrusions 250 to collapse in a controlled manner (the large distal portion 257 prevents the three-dimensional protrusion 250 from flopping over and pushing back into the original plane of the topsheet/acquisition layer laminate 245). Thus, the three-dimensional protrusions 250 are free of a hinge structure that would otherwise permit them to fold to the side when compressed.

In the area of the three-dimensional protrusions, the topsheet 24 and/or acquisition layer 52 may comprise one or more interruptions. The formation of the one or more interruptions may be due to the properties of the topsheet 24 and acquisition layer 52. The topsheet 24 may be less extensible with regard to fiber mobility and/or fiber extensibility than the acquisition layer 52 or vice versa such that a hole starts to form in the topsheet 24 and/or acquisition layer 52.

As shown in FIG. 17C, the acquisition layer 52 may be interrupted in the area of the three-dimensional protrusion 250 of the topsheet/acquisition layer laminate 245.

Generally, the acquisition layer 52 may have a lower extensibility than the topsheet 24. In such cases, the acquisition layer 52 may start to rupture and form an interruption, i.e. the fibers composing the acquisition layer 52 may be less extensible and/or mobile than the fibers composing the topsheet 24.

FIG. 18A-FIG. 18E shows alternatives how a plurality of three-dimensional protrusions 250, e.g. bulbous-shaped protrusions, may protrude from the acquisition layer 52 to the topsheet 24 of the topsheet/acquisition layer laminate 245.

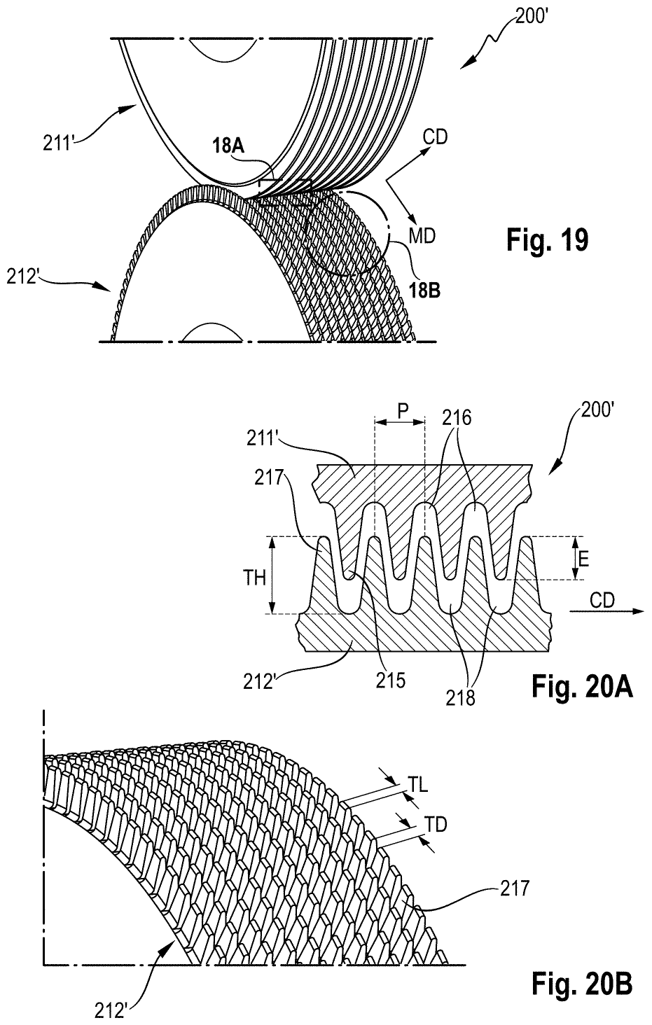

Alternatively, the topsheet/acquisition layer laminate may be obtainable according to a process which comprises the step of providing a first and second intermeshing roll (211′, 212′) as shown in FIGS. 19, 20A and 20B.

The first intermeshing roll 211′ of an apparatus 200′ may comprise a plurality of ridges 215 and corresponding grooves 216 which extend unbroken substantially about a circumference of the first intermeshing roll 211′.

The second intermeshing roll 212′ may comprise a plurality of rows of circumferentially-extending ridges that have been modified to be rows of circumferentially-spaced teeth 217 and corresponding grooves 218, wherein the plurality of rows of circumferentially-spaced teeth 217 extend in spaced relationship about at least a portion of the second intermeshing roll 212′.

The topsheet 24 and acquisition layer 52 may be intermeshed together between the first and second intermeshing rolls (211′, 212′) such that the ridges 215 of the first intermeshing roll 211′ extend into the grooves 218 of the second intermeshing roll 212′ and the teeth 217 of the second intermeshing roll 212′ extend into the grooves 216 of the first intermeshing roll 211′ to form the topsheet/acquisition layer laminate 245. Hence, a plurality of deformations comprising three-dimensional protrusions 250 is obtained.

The three-dimensional protrusions 250 of the topsheet/acquisition layer laminate 245 may be only formed where the topsheet 24 overlaps the acquisition layer 52 in the topsheet/acquisition layer laminate 245.

The first and second intermeshing roll (211′, 212′) may be further defined by a tooth height TH, a pitch P and a depth of engagement E as shown in FIG. 20A. The tooth height TH may be measured from a surface of the second intermeshing roll 172 to a tip of a tooth 177. The tooth height TH may range from 0.5 mm to 10 mm or from 0.5 mm to 5 mm.

The pitch P may be defined as a tooth-to-tooth spacing which is measured from a tip of a first tooth to a tip of a second tooth of the second intermeshing roll 172. The first and second tooth of the second intermeshing roll 212′ may be located in the cross-machine direction. The pitch P may range from 1 mm to 10 mm or from 1 mm to 5 mm.

The depth of engagement E is a measure of how much the first and second intermeshing rolls (211′, 212′) are engaging with each other. The depth of engagement E may be measured from a tip of a ridge 215 to a tip of a tooth 217 which is located next to the ridge 215 in the cross-machine direction. The depth of engagement E may range from 0.5 mm to 10 mm or from 0.5 mm to 5 mm or from 1 to 4 mm.

Each tooth 217 of the second intermeshing roll 212′ may be defined by a circumferential tooth length TL and a tooth distance TD, as shown in FIGS. 19 and 20B. The circumferential tooth length TL may be measured from a leading edge to a trailing edge at a tooth tip. The tooth length TL may range from 0.5 mm to 10 mm or from 0.5 mm to 4 mm or from 1 mm to 4 mm.

Each tooth is separated from one another circumferentially by the tooth distance TD. The tooth distance TD may be measured from a leading edge of a first tooth to a trailing edge of a second tooth. The first and second teeth of the second intermeshing roll 172 may be on the same circumference in the machine direction. The tooth distance TD may range from 0.5 mm to 10 mm or from 0.5 mm to 5 mm or from 1 mm to 3 mm.

Other orientations of the teeth 217, grooves (216, 218) and ridges 215 may be possible, e.g. in CD direction versus MD direction.

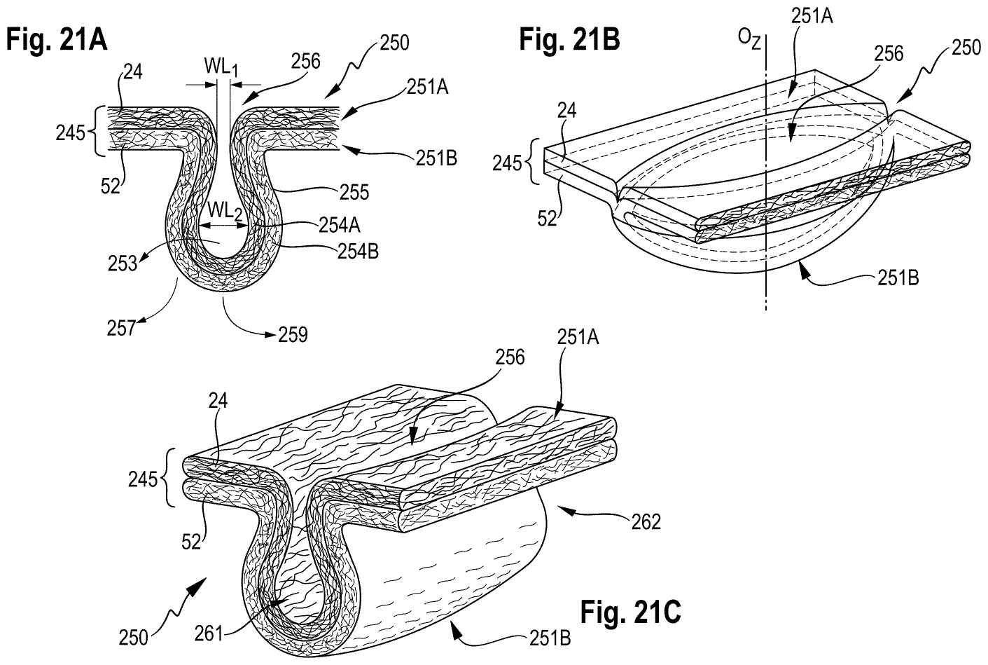

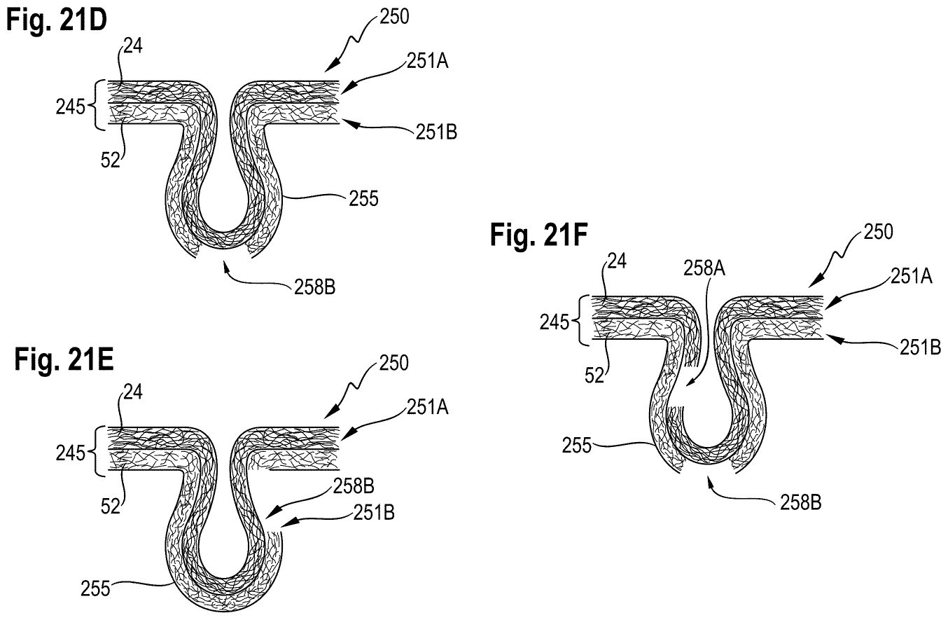

FIG. 21A-FIG. 21F shows different alternatives of three-dimensional protrusions 250. A loop-shaped protrusion may be one type of three-dimensional protrusions 250, see for example FIG. 21B. A loop-shaped protrusion may be obtained by the intermeshing process step as just described above using the apparatus 170.

Another type of three-dimensional protrusion 250 may be a tunnel-shaped loop. Generally, a tunnel-shape loop may comprise a base forming an opening and also an opening at a leading edge 261 and an opening at a trailing edge 262, see for example FIG. 21C.

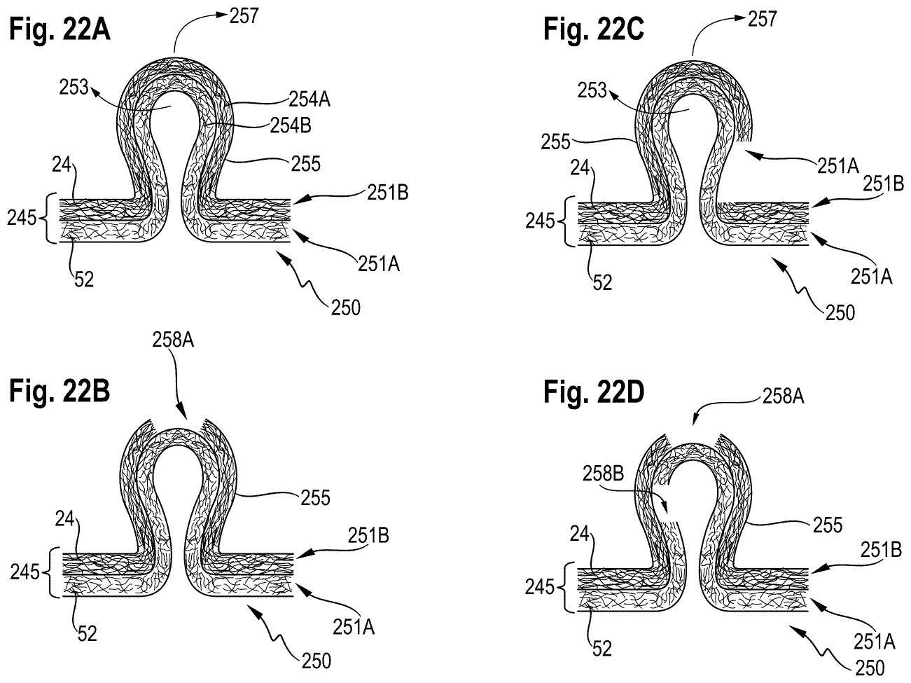

FIG. 22A-FIG. 22D shows alternatives how a plurality of three-dimensional protrusions 250, e.g. loop-shaped protrusions, may protrude from the acquisition layer 52 to the topsheet 24 of the topsheet/acquisition layer laminate 245.

### Fiber Concentration

The topsheet 24 may comprise a generally planar first region of the topsheet 24. The acquisition layer 52 may comprise a generally planar first region of the acquisition layer 52. The three-dimensional protrusions of the respective topsheet 24 and the acquisition layer 52 may comprise a plurality of discrete integral second regions. The term “generally planar” is not meant to imply any particular flatness, smoothness, or dimensionality. Thus, the first region of the topsheet 24 can include other features that provide the first region of the topsheet 24 with a topography. The first region of the acquisition layer 52 can include other features that provide the first region of the acquisition layer 52 with a topography. Such other features can include, but are not limited to small protrusions, raised network regions around the base 256 forming an opening, and other types of features. Thus, the first region of the topsheet 24 and/or the first region of the acquisition layer 52 can be generally planar when considered relative to the respective second regions. The first region of the topsheet 24 and/or the first region of the acquisition layer 52 can have any suitable plan view configuration. In some cases, the first region of the topsheet 24 and/or the first region of the acquisition layer 52 can be in the form of a continuous inter-connected network which comprises portions that surround each of the three-dimensional protrusions 250.

The side walls 259 and the area around the base 256 of the majority of the three-dimensional protrusions 250 may have a visibly significantly lower concentration of fibers per given area (which may be evidence of a lower basis weight or lower opacity) than the portions of the topsheet 24 and/or the acquisition layer 52 in the unformed first region of the respective topsheet 24 and the acquisition layer 52. The majority of the three-dimensional protrusions 250 may also have thinned fibers in the side walls 259. Thus, the fibers may have a first cross-sectional area when they are in the undeformed topsheet 24 and the acquisition layer 52, and a second cross-sectional area in the side walls 259 of the majority of the three-dimensional protrusions 250 of the topsheet/acquisition layer laminate 250, wherein the first cross-sectional area is greater than the second cross-sectional area. The side walls 259 may also comprise some broken fibers as well. The side walls 259 may comprise greater than or equal to about 30%, alternatively greater than or equal to about 50% broken fibers.

As used herein, the term “fiber concentration” has a similar meaning as basis weight, but fiber concentration refers to the number of fibers/given area, rather than g/area as in basis weight.

The topsheet/acquisition layer laminate 245 may comprise the majority of the three-dimensional protrusions 250 which are oriented with the base 256 facing upward in which the concentration of fibers at the distal end 259 of each respective topsheet 24 and the acquisition layer 52 differs between the topsheet 24 and the acquisition layer 52.

The concentration of fibers in the first region of the acquisition layer 52 and in the distal ends 259 of the majority of the three dimensional protrusions 250 may be greater than the concentration of fibers in the side walls 255 of the majority of the three dimensional protrusions 250 in the acquisition layer 52

The concentration of fibers in the first region of the topsheet 24 and in the distal ends 259 of the majority of the three dimensional protrusions 250 may be greater than the concentration of fibers in the side walls 255 of the majority of the three dimensional protrusions 250 in the topsheet 24.

Alternatively, the concentration of fibers in the first region of the acquisition layer 52 may be greater than the concentration of fibers in the side walls 255 of the majority of the three-dimensional protrusions 250 in the acquisition layer 52, and the concentration of fibers in the side walls 255 of the majority of the three-dimensional protrusions 250 in the acquisition layer 52 may be greater than the concentration of fibers forming the distal ends 259 of the majority of the three-dimensional protrusions 250 in the acquisition layer 52.

The concentration of fibers in the first region of the acquisition layer 52 may be greater than the concentration of fibers in the distal ends 259 of the majority of the three dimensional protrusions 250 in the acquisition layer 52, and the concentration of fibers in the first region of the topsheet 24 and the distal ends 259 of the majority of the three dimensional protrusions 250 may be greater than the concentration of fibers in the side walls 255 of the majority of the three dimensional protrusions 250 in the topsheet 24.

A portion of the fibers that form the first region fibers in the acquisition layer 52 and/or the topsheet 24 may comprise thermal point bonds, and the portion of the fibers in the acquisition layer 52 and/or the topsheet 24 forming the side walls 255 and distal ends 259 of the majority of the three-dimensional protrusions 250 may be substantially free of thermal point bonds. In at least some of the three-dimensional protrusions, at least some of the fibers in the acquisition layer 52 and/or the topsheet 24 may form a nest or circle around the perimeter of the three-dimensional protrusion 250 at the transition between the side wall 255 and the base 256 of the three-dimensional protrusion 250.

In some cases, the topsheet 24 or the acquisition layer 52 may have a plurality of bonds (such as thermal point bonds) therein to hold the fibers together. Any such bonds are typically present in the precursor materials from which the respective topsheet 24 or the acquisition layer 52 are formed.

Forming three-dimensional protrusions 250 in the topsheet/acquisition layer laminate 245 may also affect the bonds (thermal point bonds) within the topsheet 24 and/or the acquisition layer 52.

The bonds within the distal end 259 of the three-dimensional protrusions 250 may remain intact (not be disrupted) by the mechanical deformation process that formed the three-dimensional protrusions 250. In the side walls 255 of the three-dimensional protrusions 250, however, the bonds originally present in the precursor topsheet 24 and/or the acquisition layer 52 may be disrupted. When it is said that the bonds may be disrupted, this can take several forms. The bonds can be broken and leave remnants of a bond. In other cases, such as where the precursor materials of the respective topsheet 24 or the acquisition layer 52 is underbonded, the fibers can disentangle from a lightly formed bond site (similar to untying a bow), and the bond site will essentially disappear. In some cases, after the mechanical deformation process, the side walls 255 of the majority of the three-dimensional protrusions 250 may be substantially free (or completely free) of thermal point bonds.

The bonds within the first region of the topsheet 24 and the distal end 259 of the three-dimensional protrusions 250 may remain intact. In the side walls 255 of the three-dimensional protrusions 250, however, the bonds originally present in the precursor topsheet 24 may be disrupted such that the side walls 255 are substantially free of thermal point bonds. Such a topsheet 24 could be combined with an acquisition layer 52 in which the concentration of fibers within the topsheet 24 in the first region and the distal end 259 of the three-dimensional protrusions 250 is also greater than the concentration of fibers in the side walls 255 of the three-dimensional protrusions 250.

The acquisition layer 52 may have thermal point bonds within the first region of the acquisition layer 52 and the distal end 259 of the three-dimensional protrusions 250 that remain intact. In the side walls 255 of the three-dimensional protrusions 250, however, the bonds originally present in the precursor acquisition layer 52 comprising the acquisition layer 52 may be disrupted such that the side walls 255 of the acquisition layer 52 are substantially free of thermal point bonds. In other cases, the thermal point bonds in the acquisition layer 52 at the distal end 259 of the three-dimensional protrusions 250 may also be disrupted so that the distal end 259 of at least some of the three-dimensional protrusions 250 are substantially or completely free of thermal point bonds.

### A Three Dimensional Substrate and a Tissue Layer

The absorbent article 20 comprises a three dimensional substrate 240, which may be a portion or all of the topsheet 24, or which may be preferably a topsheet/acquisition layer laminate 245. The absorbent article 20 comprises a tissue layer 17, an absorbent core 28 and a backsheet 25.

The absorbent core 28 may comprise an absorbent material 60 which comprises from 80% to substantially 100% of superabsorbent polymers by total weight of the absorbent material. The tissue layer 17 is located between the three-dimensional substrate 240 and the absorbent core 28. The tissue layer 17 comprises a wet-laid three-dimensional fibrous substrate 120 as set out more below in detail.

The absorbent article 20 may comprise an acquisition layer 52 beneath the three-dimensional substrate 240 and a dry-laid fibrous structure 54. The tissue layer 17 has a first and second surface 171, 172. The dry-laid fibrous structure 54 may be located on the first surface 171 of the tissue layer 17 such that the second surface 172 of the tissue layer 17 is facing towards the acquisition layer 52 or the backsheet 25.

Alternatively, the absorbent article 20 comprises a three dimensional substrate 240 which may be preferably a topsheet/acquisition layer laminate 245. The absorbent article 20 may comprise a topsheet 24 comprising a plurality of fibers and an acquisition layer 52 comprising a plurality of fibers. The topsheet/acquisition layer laminate 245 may comprise the topsheet 24 and the acquisition layer which are in a face to face relationship. The topsheet/acquisition layer laminate 245 may comprise three-dimensional protrusions 250 extending from a plane of the laminate 245.

The absorbent article 20 may further comprise a dry-laid fibrous structure 54. The three-dimensional substrate 240 such as the topsheet/acquisition layer laminate 245 may be produced at a particular location in the process setup. Hence, the topsheet/acquisition layer laminate 245 might be not available to carry the dry-laid fibers 540 of the distribution layer 54 at the desired location of the process.

According to the method used for making the three-dimensional substrate 10, such as the ones described above to make the topsheet/acquisition layer laminate 245, when the topsheet 24 and acquisition layer 52 are mechanically deformed together, holes might unintentionally occur. When the distribution layer 54 comprises dry-laid fibers 540 of a dry-laid fibrous structure, the dry-laid fibers 540 may pass through the unintentional holes formed at the three-dimensional substrate 240 and contact undesirably the skin of the wearer. It may be desirable to prevent that dry-laid fibers 540 can pass through the unintentional holes of the three-dimensional substrate 240.

The tissue layer 17 has a first and second surface 171, 172. The dry-laid fibrous structure 54 may be located on the first surface 171 of the tissue layer 17 such that the second surface 172 of the tissue layer 17 is facing towards the topsheet/acquisition layer laminate 245 or the backsheet 25.

A tissue layer 17 may be disposed between the three-dimensional substrate 240 and the dry-laid fibers 540, as shown in FIG. 8. The tissue layer 17 may act as a barrier layer to impede the dry-laid fibers 540 from passing through the holes of the three-dimensional substrate 240 unintentionally formed when making the three-dimensional substrate 240. Also, the tissue layer 17 may help the transfer of the liquid bodily exudates from the three-dimensional substrate 240 to the dry-laid fibrous structure.

The tissue layer 17 comprises a wet-laid three-dimensional fibrous substrate 120 which comprise wet-laid fibers. The wet-laid fibers may comprise cellulosic fibers, such as pulp fibers. The wet-laid three-dimensional fibrous substrate 120 can provide a natural hydrophilic material for capillary connectivity between the distribution layer 54 and the absorbent core 28 when the second surface 172 of the tissue layer 17 is facing towards the backsheet 25. Alternatively, the wet-laid three-dimensional fibrous substrate 120 can provide a natural hydrophilic material for capillary connectivity between the three-dimensional substrate 240 and the distribution layer 54 when the second surface 172 of the tissue layer 17 is facing towards the three-dimensional substrate 240. Hence, the tissue layer 17 can help dewatering the topsheet 24 of the absorbent article 20 by providing a capillary connectivity between the different layers of the absorbent article 20. The tissue layer 17 comprising the wet-laid three-dimensional fibrous substrate 120 comprising at least 80% pulp fibers by weight of the wet-laid three-dimensional fibrous substrate 120 can provide some absorbency properties which can improve the fluid handling properties of the absorbent article 20. The tissue layer 17 can help to reduce the amount of the dry-laid fibers 540 of the distribution layer 54.

The amount of the dry-laid fibers 540 of the distribution layer 54 can be reduced in the back region 38 or in the front region 36, and at least partially in the crotch region 37 of the absorbent article 20. In that case, the tissue layer 17 may have opacity which differs from the top side 16 of the core wrap 160 of the absorbent core 28 in order to cover the portion of the top side 16 of the core wrap 160 facing the portion of the distribution layer 54 having a reduced amount of distribution material.

The topsheet/acquisition layer laminate 245 may comprise a plurality of three-dimensional protrusions 250 which may extend towards the distribution layer 54 (see also FIG. 8). When the three-dimensional protrusions 250 extend towards the distribution layer 54, the area of contact between the acquisition layer 52 of the topsheet/acquisition layer laminate 245 and the underneath distribution layer 54 is improved. The distribution layer 54 will follow the shape of the three-dimensional protrusions 250. Hence, the transfer of the liquid bodily exudates from the topsheet/acquisition layer laminate 245 to the distribution layer 54 can be increased.

A first surface 171 of the tissue layer 17 may be attached at or adjacent to its longitudinal edges to the absorbent core 28. Hence, when the tissue layer 17 is disposed between the three-dimensional substrate 240, e.g. the topsheet/acquisition layer laminate 245 and the dry-laid fibrous structure, and first surface 171 of the tissue layer 17 is attached to the absorbent core 28, the dry-laid fibers 540 of the dry-laid fibrous structure may be not able to escape between the tissue layer 17 and the absorbent core 28. The attachment of the tissue layer 17 to the longitudinal edges of the absorbent core 28 may include a uniform continuous layer of adhesive 173, a discontinuous patterned application of adhesive or an array of separate lines, spirals, or spots of adhesive.

Alternatively, the tissue layer 17 may be disposed between the dry-laid fibrous structure and the absorbent core 28, as shown in FIG. 9. Hence, the tissue layer may help to distribute and transfer of the liquid bodily exudates from the distribution layer 54 to the absorbent core 28, as shown in FIG. 9, which enables more efficient use of the absorbent core 28.

The tissue layer 17 may be attached at or adjacent to its longitudinal edges to the first surface of the three-dimensional substrate 240. Hence, when the tissue layer 17 is disposed between the dry-laid fibrous structure and the absorbent core 28, and the tissue layer 17 is attached to the first surface of the three-dimensional substrate 240, the dry-laid fibers 540 of the dry-laid fibrous structure may be not able to escape between the three-dimensional substrate 240 and the tissue layer 17. The attachment of the tissue layer 17 to the longitudinal edges to the first surface of three-dimensional substrate 240 may include a uniform continuous layer of adhesive, a discontinuous patterned application of adhesive or an array of separate lines, spirals, or spots of adhesive.

The tissue layer 17 may be wider and longer than the distribution layer 54. The tissue layer 17 can help preventing the dry-laid fibers 540 getting to the skin of the wearer when the distribution layer 54 comprises the dry-laid fibrous structure and if the three-dimensional substrate 240 comprises some holes.

The tissue layer 17 may have been activated through a ring rolling process, i.e. a mechanical deformation. Alternatively, the tissue layer 17 may have been activated through a process comprising the step of providing a first and second intermeshing roll (211′, 212′), as described above.

The tissue layer 17 may be activated according to the process which comprises the step of providing a first and second intermeshing roll (211′, 212′) as shown in FIGS. 19, 20A and 20B.

The first intermeshing roll 211′ of an apparatus 200′ may comprise a plurality of ridges 215 and corresponding grooves 216 which extend unbroken substantially about a circumference of the first intermeshing roll 211′. The second intermeshing roll 212′ may comprise a plurality of rows of circumferentially-extending ridges that have been modified to be rows of circumferentially-spaced teeth 217 and corresponding grooves 218, wherein the plurality of rows of circumferentially-spaced teeth 217 extend in spaced relationship about at least a portion of the second intermeshing roll 212′. The tissue layer 17 may be intermeshed between the first and second intermeshing rolls (211′, 212′) such that the ridges 215 of the first intermeshing roll 211′ extend into the grooves 218 of the second intermeshing roll 212′ and the teeth 217 of the second intermeshing roll 212′ extend into the grooves 216 of the first intermeshing roll 211′ to activate the tissue layer 17. The activation of the tissue layer 17 can help to provide softness and pliability for the tissue layer 17, which can improve the fit of the absorbent article and the comfort for the wearer.

The first surface 171 of the tissue layer 17 may comprise one or more substantially dry-laid fiber free area(s) wherein each dry-laid fiber free area comprises less than 2% of dry-laid fibers by total weight of dry-laid fibers. When the absorbent core 28 comprises one or more substantially absorbent material free area(s) 26 through which a portion of the top side of the core wrap 160 is attached by one or more core wrap bond(s) to a portion of the bottom side of the core wrap, each dry-laid fiber free area may be substantially parallel to the substantially absorbent material free area(s) 26. This can help to reinforce this impression of depth.

The tissue layer 17 may have a total area. The dry-laid fibrous structure which is located on the first surface of the tissue layer may have a total area; and the total area of the dry-laid fibrous structure may be less than the total area of the tissue layer. For example, the length of the distribution layer 54 may be shorter than the length of the tissue layer 17.

The tissue layer 17 may be colored and/or comprise one or more graphic zones. Color may be imparted to the tissue layer 17 by flexographic multicolor printing. The term “color” does not include “white” pigments such as TiO₂ which are typically added to the layers of conventional absorbent articles to impart them with a white appearance.

When viewing the absorbent article 20 from the topsheet 24, the colored tissue layer 17 may provide to a caregiver an enhanced impression of depth to support to the impression given by the three-dimensional protrusions 250 as such, as long as the colored tissue layer 17 are visible from the topsheet 24. Hence, a colored tissue layer 17 can support the caregiver's perception that the absorbent article 20 is well able to absorb the liquid bodily exudates.

Also, the tissue layer 17 can provide an adequate substrate to get the graphics printed accurately on the tissue layer 17. As the graphics of the tissue layer can be visible through the three-dimensional substrate 240. Making the graphics visible to the consumers can further provide to a caregiver an enhanced impression of depth to support to the impression given by the three-dimensional protrusions 250 of the three-dimensional substrate 240.

The topsheet 24 comprising the three-dimensional substrate 240, or the topsheet 24 and/or acquisition layer 52 of the topsheet/acquisition layer laminate 245 may be colored, for the same reasons.

Also, the tissue layer 17 may comprise some relative small sized holes such that the dry-laid fibers 540 of the distribution layer 54 may partially pass through the holes of the tissue layer. Hence, the dry-laid fibers 540 can entangle and contact the acquisition layer 52 of the topsheet/acquisition layer laminate 245. The tissue layer 17 may comprise holes having a size from 0.02 mm to 10 mm

### Wet-Laid Three-Dimensional Fibrous Substrate

The tissue layer 17 comprises a wet-laid three-dimensional fibrous substrate 120. At least a portion of, or all of the tissue layer 17 may comprise one or more wet-laid three-dimensional fibrous substrates or more than one layer of a wet-laid three-dimensional fibrous substrate.

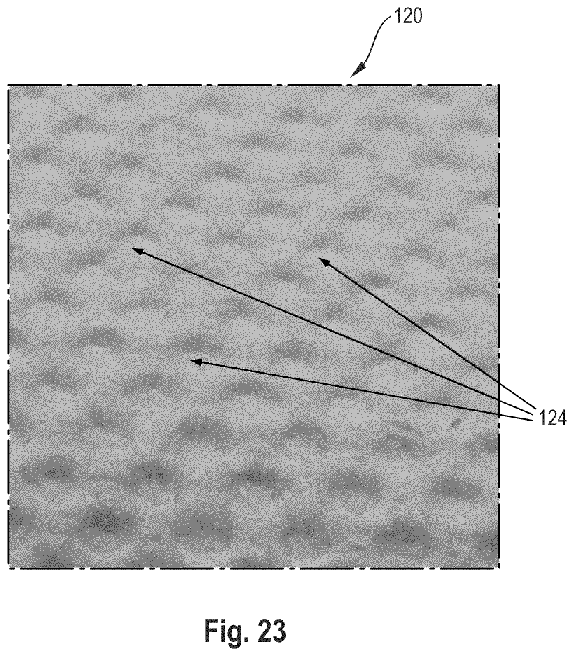

The wet-laid three dimensional fibrous substrate 120 of the tissue layer 17 comprises at least 80% pulp fibers by weight of the wet-laid three dimensional substrate 120 (see FIG. 23).

The wet-laid three dimensional fibrous substrate 120 of the tissue layer 17 may comprise at least 90% pulp fibers by weight of the wet-laid three dimensional substrate 120.

The wet-laid three dimensional fibrous substrate 120 of the tissue layer 17 may comprise from 70% to 100%, or at least 80%, or at least 85%, or at least 90%, or at least 95%, or at least 99% pulp fibers by weight of the wet-laid three-dimensional fibrous substrate 120.

The tissue layer 17 comprises a wet-laid three-dimensional fibrous substrate 120 which comprise wet-laid fibers. The wet-laid fibers may comprise cellulosic fibers, such as pulp fibers. The wet-laid fibers may be produced by forming a predominantly aqueous slurry comprising 90% to 99.9% water or other suitable fluid or liquid. In one form, the non-aqueous component of the slurry used to make the wet-laid and/or wet-formed fibers may comprise from 1% to 95% or 5% to 80% of cellulosic fibers, such as eucalyptus fibers, by weight of the non-aqueous components of the slurry. In another form, the non-aqueous components may comprise from 8% to 60% of cellulosic fibers, such as eucalyptus fibers, by weight of the non-aqueous components of the slurry, or from 15% to 30% of cellulosic fibers, such as eucalyptus fibers, by weight of the non-aqueous component of the slurry. In some instances, the slurry may comprise 45% to 60% of Northern Softwood Kraft fibers with up to 20% Southern Softwood Kraft co-refined together, 25% to 35% unrefined Eucalyptus fibers and from 5% to 30% of either repulped product broke or thermo-mechanical pulp. Any other suitable cellulosic fibers and/or combinations thereof within the knowledge of those of skill in the art may also be used.

The wet-laid fibers of the wet-laid three-dimensional fibrous substrate 120 may comprise a mixture of at least two different materials. At least one of the materials may comprise a non-naturally occurring fiber, such as a polypropylene fiber or a polyolefin fiber, for example, and at least one other material, different from the first material, comprising a solid additive, such as another fiber and/or a particulate, for example.

Synthetic fibers useful herein may comprise any suitable material, such as, but not limited to polymers, those selected from the group consisting of polyesters, polypropylenes, polyethylenes, polyethers, polyamides, polyhydroxyalkanoates, polysaccharides, and combinations thereof. More specifically, the material of the polymer segment may be selected from the group consisting of poly(ethylene terephthalate), poly(butylene terephthalate), poly(1,4-cyclohexylenedimethylene terephthalate), isophthalic acid copolymers (e.g., terephthalate cyclohexylene-dimethylene isophthalate copolymer), ethylene glycol copolymers (e.g., ethylene terephthalate cyclohexylene-dimethylene copolymer), polycaprolactone, poly(hydroxyl ether ester), poly(hydroxyl ether amide), polyesteramide, poly(lactic acid), polyhydroxybutyrate, and combinations thereof.

Further, the synthetic fibers may be a single component fibers (i.e., single synthetic material or a mixture to make up the entire fiber), multi-component fibers, such as bi-component fibers (i.e., the fiber is divided into regions, the regions including two or more different synthetic materials or mixtures thereof), and combinations thereof. Nonlimiting examples of suitable bicomponent fibers are fibers made of copolymers of polyester (polyethylene terephthalate/isophtalate/polyester (polyethylene terephthalate) otherwise known as “CoPET/PET” fibers, which are commercially available from Fiber Innovation Technology, Inc., Johnson City, Tenn.

### Non-Wood Pulp Fibers

The pulp fibers may also comprise non-wood fibers. Non-wood fibers may comprise fibers made from polymers, specifically hydroxyl polymers. Non-limiting examples of suitable hydroxyl polymers include polyvinyl alcohol, starch, starch derivatives, chitosan, chitosan derivatives, cellulose derivatives, gums, arabinans, galactans, and combinations thereof. Additionally, other synthetic fibers such as rayon, polyethylene, and polypropylene fibers can be used within the scope of the present disclosure.

Non-wood pulp fibers may also comprise fibers that comprise processed residuals from agricultural crops such as wheat straw, wetland non-tree plants such as bulrush, aquatic plants such as water hyacinth, microalgae such as Spirulina and macroalgae seaweeds such as red or brown algae. Examples of non-wood natural materials include, but are not limited to, wheat straw, rice straw, flax, bamboo, cotton, jute, hemp, sisal, bagasse, hesperaloe, switchgrass, miscanthus, marine or fresh water algae/seaweeds, and combinations thereof.

### Optional Ingredients

To enhance permanent wet strength of one or more wet-laid three-dimensional fibrous substrates 120 of the tissue layer 17, cationic wet strength resins may be added to the papermaking furnish or to the embryonic web.

The wet-laid three-dimensional fibrous substrate 120 made of wet-laid fibers may comprise one or more cationic wet strength resins selected from the group consisting of a base activated epoxide polyamide epichlorohydrin resin, an urea-formaldehyde resin, a melamine formaldehyde resin, a polyamide-epichlorohydrin resin, a polyethyleneimine resin, a polyacrylamide resin, a dialdehyde starch and mixtures thereof.

The cationic wet strength resins may comprise cationic water soluble resins. These resins may improve wet strength in a fibrous substrate. This resin may improve either temporary or permanent wet strength to the fibrous substrate. KYMENE® resins obtainable from Hercules Inc., Wilmington, Del. may be used, including KYMENE® 736 which is a polyethyleneimine (PEI) wet strength polymer. It is believed that the PEI may improve wet strength by ionic bonding with the pulps carboxyl sites. KYMENE® 557LX is polyamide epichlorohydrin (PAE) wet strength polymer. It is believed that the PAE contains cationic sites that may lead to resin retention by forming an ionic bond with the carboxyl sites on the pulp. KYMENE® 450 is a base activated epoxide polyamide epichlorohydrin polymer. It is theorized that like 557LX the resin attaches itself ionically to the pulps' carboxyl sites via the epoxide groups of 557LX. KYMENE® 2064 is also a base activated epoxide polyamide epichlorohydrin polymer. It is theorized that KYMENE® 2064 may improve its wet strength by the same mechanism as KYMENE® 450. KYMENE® 2064 differs in that the polymer backbond contains more epoxide functional groups than does KYMENE® 450. Mixtures of the foregoing may be used. Other suitable types of such resins include urea-formaldehyde resins, melamine formaldehyde resins, polyamide-epichlorohydrin resins, polyethyleneimine resins, polyacrylamide resins, dialdehyde starches, and mixtures thereof.

### The Structure of the Wet-Laid Three-Dimensional Fibrous Substrate of the Tissue Layer

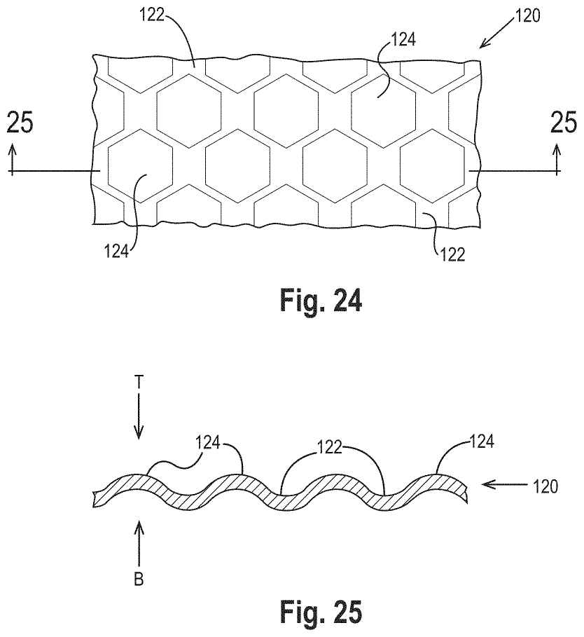

Referring to FIGS. 24 and 25, a wet-laid three-dimensional fibrous substrate 120 is formed that has at least a first region which is a continuous network region 122 and a second region which is a plurality of discrete zones 124.

The continuous network region 122 may be raised or indented relative to the plurality of discrete zones 124. Stated another way, the continuous network region 122 may form a high density zone and the plurality of discrete zones 124 may form a low density zone or the continuous network region 122 may form a low density zone and the plurality of discrete zones 124 may form a high density zone. Regardless of whether each of the regions or discrete zones is high or low density, the plurality of discrete zones 124 are dispersed throughout and/or formed within the continuous network region 122.

The continuous network region 122 and the plurality of discrete zones 124 have a common intensive property. Alternatively, the continuous network region 122 and the plurality of discrete zones 124 have a first and a second intensive properties which differ from each other.

The common intensive property of the continuous network region 122 has a first value. The common intensive property of the plurality of discrete zones 124 has a second value. The first value is different than the second value.

The common intensive property is selected from the group consisting of basis weight, dry caliper, opacity, average density, elevation and combinations thereof.

The continuous network region 122 may have a first average density and the plurality of discrete zones 124 may have a second, different average density, according to the Average Density Test Method herein. Although referred to herein as a “continuous network region”, as an example, it will be understood that the “network region” may be substantially continuous or substantially semi-continuous. Herein the network region will be referred to as “continuous” as an example and not to limit the present disclosure.

The average density of the continuous network region 122 may be higher than the average density of the plurality of discrete zones 124. FIG. 24 illustrates a plan view a portion of the wet-laid three-dimensional fibrous substrate 120 where the continuous network region 122 is illustrated as defining hexagons, although it is to be understood that other preselected patterns may also be used.

FIG. 25 is a cross-sectional view of the wet-laid three-dimensional fibrous substrate 120 taken along line 25-25 of FIG. 24. As can be seen from the example of FIG. 25, the continuous network region 122 is essentially monoplanar. The plurality of discrete zones 124 are dispersed throughout the entire continuous network region 122 and essentially each discrete zone 124 is encircled by the continuous network region 122. The shape of the discrete zones 124 may be defined by the continuous network region 122. As shown in FIG. 25, the discrete zones 124, appear to protrude from the plane formed by continuous network region 122 toward an imaginary observer looking in the direction of arrow T of FIG. 25. When viewed by an imaginary observer looking in the direction indicated by arrow B of FIG. 25, the plurality of discrete zones 124 may comprise arcuately shaped voids which appear to be cavities or dimples.

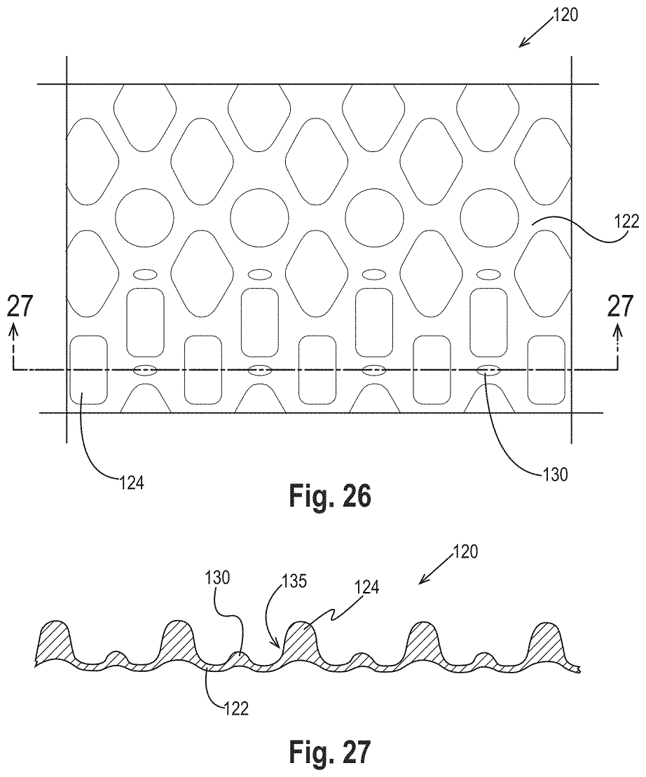

Referring to FIGS. 26 and 27, the continuous network region 122 and the plurality of discrete zones 124 of the wet-laid three-dimensional fibrous substrate 120 may also differentiate in their respective micro-geometry. In the example of FIGS. 26 and 27, the continuous network region 122 forms a first plane (knuckles or high density regions) at a first elevation when the wet-laid three-dimensional fibrous substrate 120 is disposed on a flat surface and the plurality of discrete zones 124 are dispersed throughout the continuous network region 122. These discrete zones 124 may, comprise discrete protuberances, or “pillows,” (or low density regions) outwardly extending from the continuous network region 122 to form a second elevation greater than the first elevation, relative to the first plane. Alternatively, the continuous network region 122 may comprise the pillows or low density regions (higher elevation) and the plurality of discrete zones 124 may comprise the knuckles or high density regions (lower elevation) depending on how the papermaking belt is formed. It is to be understood that pillows and/or knuckles may also comprise a continuous pattern, a substantially continuous pattern, or a substantially semi-continuous pattern.

Referring again to FIGS. 26 and 27, the wet-laid three-dimensional fibrous substrate 120 may comprise a third region 130 having at least one intensive property that is common with and differs in value from the intensive property of the continuous network region 122 and the intensive property of the plurality of discrete zones 124. For example, the continuous network region 122 may have a common intensive property having a first value, the plurality of discrete zones 124 may have the common intensive property having a second value, and the third region 130 may have the common intensive property having a third value, wherein the first value may be different from the second value, and the third value may be different from the second value and the first value. The common intensive property may be any of those specified herein.

The third region 130 may comprise one or more transition regions 135 (see FIG. 27) located between the continuous network region 122 and the plurality of discrete zones 124. The transition region 135 is the area or region between which the continuous network region 122 and the plurality of discrete zones 124 transition. Stated another way, the transition regions 135 are positioned intermediate the continuous network region 122 and the plurality of discrete zones 124. The continuous network region 122, the plurality of discrete zones 124, and the plurality of transition regions 135 may each have a first and/or second common intensive property. The first and/or second common intensive property of the continuous network region 122 may have a first value, the first and/or second common intensive property of the plurality of discrete zones 124 may have a second value, and the first and/or second common intensive property of the plurality of transition regions 135 may have a third value. The first and/or second common intensive property may be any of those specified herein, such as average density and basis weight, for example.

When the wet-laid three-dimensional fibrous substrate 120 comprising at least three differential regions 122, 124, 130, as described herein, is disposed on a horizontal reference plane (e.g., the X-Y plane), the first region 122 may define a plane having a first elevation, and the second region 124 may extend therefrom to define a second elevation. One form is contemplated, in which the third region 130 defines a third elevation, wherein at least one of the first, second, and third elevations is different from at least one of the other elevations. For example, the third elevation can be intermediate the first and second elevations. It is to be noted that, in the alternative, the first region 122 may have the second elevation (highest) and the second region 124 may have the first elevation (lowest). The third region 130 may exist at an elevation intermediate the second and first elevations. The transitions regions may also exist in elevation intermediate any of the first, second and third regions.

Suitable fibrous substrates having a continuous network region and a plurality of discrete zones may have predetermined elevations. For example, in certain instances, one of the continuous network region or the plurality of discrete zones may have an elevation from 50 microns to 5000 microns; one of the continuous network region or the plurality of discrete zones may have an elevation from 100 microns to 2000 microns; or one of the continuous network region or the plurality of discrete zones may have an elevation from 150 microns to 1500 microns, according to the Topographic Measurements of Differential Density Fibrous Substrates Test herein.

The following Table 1 shows, without limitation, some possible combinations of forms of the wet-laid three-dimensional fibrous substrate 120 comprising at least three regions having differential (i.e., high, medium, or low) intensive properties.

TABLE 1
INTENSIVE PROPERTIES

| HIGH | MEDIUM | LOW |
|---|---|---|
| Continuous | Discontinuous | Discontinuous |
| Continuous | Discontinuous | — |
| Continuous | — | Discontinuous |
| Semi-continuous | Semi-continuous | Semi-continuous |
| Semi-continuous | Semi-continuous | Discontinuous |
| Semi-continuous | Semi-continuous | — |
| Semi-continuous | Discontinuous | Semi-continuous |
| Semi-continuous | Discontinuous | Discontinuous |
| Semi-continuous | — | Semi-continuous |
| Discontinuous | Continuous | Discontinuous |
| Discontinuous | Continuous | — |
| Discontinuous | Semi-continuous | Semi-continuous |
| Discontinuous | Semi-continuous | Discontinuous |
| Discontinuous | Discontinuous | Continuous |
| Discontinuous | Discontinuous | Semi-continuous |
| Discontinuous | Discontinuous | Discontinuous |
| Discontinuous | — | Continuous |
| — | Continuous | Discontinuous |
| — | Semi-continuous | Semi-continuous |
| — | Discontinuous | Continuous |

Suitable wet-laid three-dimensional fibrous substrate 120 as described herein may have continuous network regions and a plurality of discrete zones having different (e.g., not the same) average densities. The average density for either the continuous network region or the plurality of discrete zones may be from 0.05 g/cm³ to 0.80 g/cm³, from 0.10 g/cm³ to 0.50 g/cm³, or from 0.15 g/cm³ to 0.40 g/cm³, according to the Average Density Test Method herein.

The average density of the continuous network region may be from 0.05 g/cm³ to 0.15 g/cm³ and the average density of the plurality of discrete zones may be from 0.15 g/cm³ to 0.80 g/cm³; the average density of the continuous network region may be from 0.07 g/cm³ to 0.13 g/cm³ and the average density of the plurality of discrete zones may be from 0.25 g/cm³ to 0.70 g/cm³; or the average density of the continuous network region may be from 0.08 g/cm³ to 0.12 g/cm³ and the average density of the plurality of discrete zones may be from 0.40 g/cm³ to 0.60 g/cm³, according to the Average Density Test Method herein.

The continuous network region may have a first basis weight and the plurality of discrete zones may have a second, different basis weight. The continuous network region may have a first dry caliper or elevation and the plurality of discrete zones may have a second dry caliper or elevation. The first and second dry calipers or elevations may be different. In other instances, the continuous network region may have a first average density and a first basis weight, while the plurality of discrete zones may have a second average density and a second basis weight. The first and second average densities and the first and second basis weights may be different. The same may apply to the third regions 130 and the transition regions 135, either relative to each other or relative to the continuous network region or the plurality of discrete zones.

Referring to FIG. 23, the tissue layer 17 comprises a wet-laid three-dimensional fibrous substrate 120. The wet-laid three-dimensional fibrous substrate 120 may be formed from wet-laid and wet-formed fibers or filaments. A continuous network region 56 is illustrated as raised elements or pillow regions (low density regions) in FIG. 23, while a plurality of discrete zones 57 are illustrated as knuckle regions (high density regions). It will be recognized that any suitable number of layers of wet-laid three-dimensional fibrous substrate 120 may be combined to form a tissue layer 17 or a portion thereof, as is described in further detail herein.

The wet-laid three-dimensional fibrous substrate 120 of the present invention can be made on a papermaking belt. US2013/0209749A1 (Myangiro) describes a method for making a wet-laid three-dimensional fibrous substrate 120 of the present invention utilizing a papermaking belt, or “molding member”.

Referring to FIG. 27, a web of a wet-laid three-dimensional fibrous substrate 120 of the tissue layer 17 may be made through the use of a patterned papermaking belt 300 for forming three-dimensionally structured wet-laid and wet-formed webs as described in U.S. Pat. No. 4,637,859, issued Jan. 20, 1987, to Trokhan.

The wet-laid three-dimensional fibrous substrate 120 may be formed using the patterned papermaking belt 300 having the plurality of raised resin portions 58, each raised resin portion 58 forming a corresponding (high density) discrete zone 124 in the fibrous substrate. The areas of the papermaking belt 300 that do not have the raised resin portions 58 form the continuous network region (low density) in the fibrous substrate. In the alternative, the raised resin portions may form a continuous network on the papermaking belt 300, which would correspondingly form a high density continuous network region in the fibrous substrate, while the areas on the papermaking belt not having the raised resin portions would form the low density discrete elements in the fibrous substrate (not illustrated).

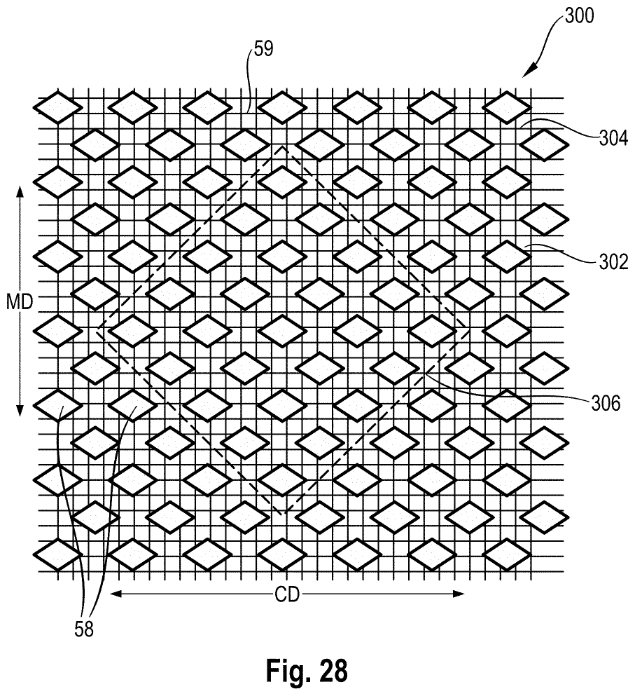

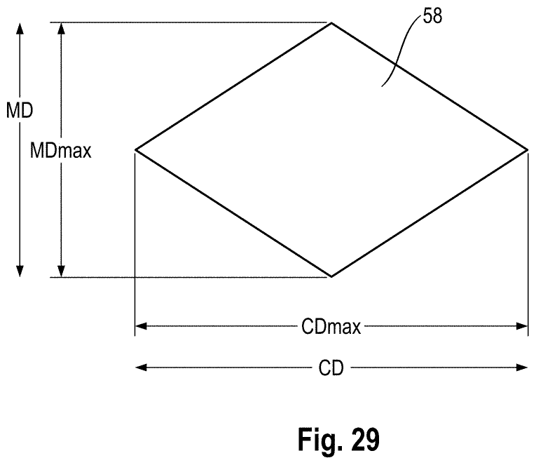

Referring again to FIG. 28, one unit 206 (shown by dashed line) of one example of a pattern of the papermaking belt 200 is illustrated. Referring to FIG. 29, a top view of an individual raised resin portion 58 that forms an individual discrete element (high density in the fibrous substrate 55 is illustrated separate from the papermaking belt 300 for clarity. The raised resin portion 58 may have any suitable shape, such as a generally elongated shape having a major axis, CDmax, and a minor axis, MDmax. The raised resin portion 58 may also have any other suitable shape, such as round, ovate, square, rectangular, trapezoidal, or any other polygonal shape. As shown in the example of FIG. 29, individual raised resin portions 58 may have a rhomboid shape. One papermaking belt 200 may have more than one shape of raised resin portions. In general, the dimensions of the discrete elements 57 of the wet-laid three-dimensional fibrous substrates 120 are determined by the dimensions of the corresponding raised portions 58 that they are formed on. That is, the wet-laid three-dimensional fibrous substrate 120 is generally formed over the three-dimensional structure of the papermaking belt 300, so that in one sense the fibers are formed over, or molded to, the raised resin portions 58. If the raised resin portions form a continuous network, then the continuous network in the wet-laid three-dimensional fibrous substrate 120 may be formed on the raised resin portions, while the discrete elements will be formed in deflection conduits intermediate portions of the raised resin portions.

The ratio of the length of axis, CDmax, to the length of axis, MDmax, may be greater than or equal to one or less than 1. Stated another way, the axis, CDmax, may be longer than, shorter than, or may have the same length as the axis, MDmax. In one form, the ratio of the length of the axis, CDmax, to the length of the axis, MDmax, may be in the range of 1 to 3 or in the range of 1 to 4 or more.

In one form, the CDmax of one raised resin portion 58 may be between 1.50 mm to 3.50 mm, 1.55 mm to 2.00 mm, or 1.53 mm and 2.29 mm, and the MDmax of one raised portion 58 may be between 0.80 mm to 2.00 mm, 1.00 mm to 1.70 mm, or 1.01 mm to 1.53 mm, specifically reciting al 0.01 mm increments within the above-specified ranges and all ranges formed therein or thereby.

Some example shapes of the discrete zones (formed by the raised portions or raised resin portions) may comprise circles, ovals, squares, rectangles, ellipses, and polygons having any suitable number of sides. There is no requirement that the discrete zones be regular polygons or that the sides of the discrete zones 124 be straight. Instead, the discrete zones may comprise curved sides, stepped sides, or other multi-level sides. The wet-laid three dimensional fibrous substrate may comprise discrete embossments. The discrete zones of the wet-laid three dimensional fibrous substrate may comprise discrete embossments.

### Physical Properties of the Tissue Layer

### Basis Weight

One or more wet-laid three-dimensional fibrous substrate 120 of the tissue layer 17 may have a basis weight in the range from 5 gsm to 150 gsm, or from 10 gsm to 100 gsm, or from 10 gsm to 60 gsm, or from 10 gsm to 50 gsm, or from 15 gsm to 50 gsm, according to the Basis Weight Test Method herein.

### Dry Caliper

A wet-laid three-dimensional fibrous substrate 120 of the tissue layer 17 may have a Dry caliper at a pressure of 2.06 kPa from 0.1 mm to 2.0 mm, or from 0.5 mm to 1.5 mm, according to the Dry Caliper Test Method herein.

### Total Dry Caliper

The tissue layer 17 may have a Total Dry caliper at a pressure of 2.06 kPa from 0.1 mm to 30.0 mm, or from 2.0 mm to 20.0 mm, from 5.0 mm to 10.0 mm according to the Dry Caliper Test Method herein.

### Wet Burst Strength

A wet-laid three-dimensional fibrous substrate 120 of the tissue layer 17 may have a Wet Burst Strength from 50 g to 500 g, or from 250 g to 350 g, or from 275 g to 325 g, or from 300 g to 350 g, according to the Wet Burst Strength Test Method herein.

### Total Dry Tensile Strength

A wet-laid three-dimensional fibrous substrate 120 of the tissue layer 17 may have a Total Dry Tensile Strength from 1000 g/in to 3000 g/in, or from 1500 g/in to 2500 g/in, or from 1700 Win to 2200 g/in, or from 1800 g/in to 2000 g/in, according to the Tensile Test Method herein.

### Geometric Mean TEA (Tensile Energy Absorption)

A wet-laid three-dimensional fibrous substrate 120 of the tissue layer 17 may have a Geometric Mean TEA from 100 g*in/in² to 500 g*in/in², from 100 g*in/in² or more, or from 150 g*in/in² or more, or from 200 g*in/in² or more, or from 300 g*in/in² or more according to the Tensile Test Method described herein.

### Geometric Mean Tensile Strength

A wet-laid three-dimensional fibrous substrate 120 of the tissue layer 17 may have a Geometric Mean Tensile Strength from 200 g/M to 1,300 g/M, or from 700 g/in to 1100 g/M, or from 800 g/M to 1000 g/M, according to the Tensile Test Method described herein.

### Geometric Mean Modulus

A wet-laid three-dimensional fibrous substrate 120 of the tissue layer 17 may have a Geometric Mean Modulus from 500 g/cm to 5000 g/cm, or from 650 g/cm to 3800 g/cm, or from 1000 g/cm to 3000 g/cm, or from 1500 g/cm to 2500 g/cm, or from 1900 g/cm to 2300 g/cm, or from 2000 g/cm to 2200 g/cm, according to the Tensile Test Method described herein.

### Geometric Mean Peak Elongation

A wet-laid three-dimensional fibrous substrate 120 of the tissue layer 17 may have a Geometric Mean Peak Elongation from 5% to 30%, or from 10% to 23%, or from 12% to 16%, or from 13% to 15%, according to the Tensile Test Method described herein.

### Elevation

Suitable wet-laid three-dimensional fibrous substrate 120 having a network region 122 and a plurality of discrete zones 124 may have predetermined elevations. For example, one of the network regions 122 or the discrete zones 124 may have an elevation from 50 microns to 5000 microns, or from 100 microns to 2000 microns, or from 150 microns to 1500 microns, according to the Topographic Measurements of Differential Density Fibrous Substrates Test Method described herein.

### Corresponding Indicia

The majority of the three-dimensional protrusions 250 of the three-dimensional substrate 240 may form a first structural indicia. The plurality of discrete zones 124 of the wet-laid three-dimensional fibrous substrate 120 of the tissue layer 17 may form a second structural indicia. The first structural indicia of the three-dimensional substrate 240 may correspond to the second structural indicia of the tissue layer 17.

The tissue layer 17 may comprise one or more structural indicia. The one or more structural indicia of the tissue layer may be one or more discrete zones 124 of the tissue layer 17. The three-dimensional substrate 240 may comprise one or more structural indicia. The one or more structural indicia of the three-dimensional substrate 240 may be one or more three-dimensional protrusions 250 of the three-dimensional substrate 240. The one or more structural indicia of the tissue layer 17 may correspond to the one or more structural indicia of the three-dimensional substrate 240.

The tissue layer 17 can provide a printable first surface 171 to provide a visual indicia with the one or more structural indicia of the tissue layer 17 in combination with the majority of the three-dimensional protrusions 250 of the three-dimensional substrate 240 forming the one or more structural indicia of the three-dimensional substrate 240. In that case, when the visual indicia of the tissue layer 17 in addition to the one or more structural indicia of the tissue layer 17 correspond to the one or more structural indicia of the three-dimensional substrate 240, this can help to reassure to the caregiver about the absorbency and dryness properties of the absorbent article 20.

One or more visual indicia of the tissue layer 17 may be selected from the group consisting of color, inks, graphics, printings, embossments, patterned adhesives and combinations thereof.

As used herein, the term “correspond” is used to describe the way or degree to which visual indicia and/or structural indicia, or characteristics thereof visually fit together or are caused to fit together.

The topsheet 24, the acquisition layer 52, and/or the tissue layer 17 may comprise one or more indicia. In other instances, more than one of these layers may comprise an indicia.

The term “indicia”, as used herein, may comprise one or more inks with pigments, adhesives with pigments, words, designs, trademarks, graphics, patterns, and/or pigmented areas, for example. The term “indicia” does not include a fully tinted or colored layer. The indicia may typically be a different color than: (1) the layer that it is printed on, positioned on, or applied to; or (2) a different color than other layers of an absorbent article 20.

The phrase a “different color” means a different shade of the same color (e.g., dark blue and light blue) or may be completely different color (e.g., blue and gray).

The indicia should be at least partially visible from either a wearer facing surface, a garment facing surface, or both of an absorbent article 20, although the indicia may not be printed on, positioned or, on applied to the wearer or garment facing surfaces of the absorbent articles 20.

The indicia may be printed on, positioned on, or applied to three-dimensional protrusions areas and non three-dimensional protrusions areas, three-dimensional protrusion areas only, or non three-dimensional protrusions areas only, for example. A three-dimensional protrusion area may comprise a portion or all of the majority of the three-dimensional protrusions 250.

The indicia may comprise a light activatable material, a liquid activatable material, a pH activatable material, a temperature activatable material, a menses activatable material, a urine activatable material, or BM activatable material, or an otherwise activatable material. These activatable materials may typically undergo a chemical reaction, or other reaction, to change the indicia from one color to a different color, from one color to a different shade of the same color, from a color that is not visually distinguishable in an absorbent article 20 to a color that is visually distinguishable in an absorbent article 20, or from a color that is visually distinguishable in an absorbent article 20 to a color that is not visually distinguishable in an absorbent article 20.

In an instance, the indicia may grow or shrink or display a graphic/not display a graphic after the indicia undergoes the reaction. In other instances, the indicia may be activated by a stress or a strain during manufacture or wear.

The indicia may be white or non-white. If the indicia is white in color, at least one layer may be non-white so that the indicia is visible from a wearer and/or garment facing surface of the absorbent articles 20, for example.

The indicia may comprise embossments, fusion bonds, or other mechanical deformations. In other instances the indicia may at least partially overlap embossments, fusion bonds, or other mechanical deformations.

In some instances, the indicia may be formed within either a sheath or a core of bicomponent fibers. For example, a core may be white, while a sheath may be blue, or vice versa.

The indicia may be on, positioned on, formed on, formed with, printed on, or applied to all of, or part of, a certain layer. The indicia may also be on, positioned on, formed on, formed with, printed on, or applied to one or more layers, or on all suitable layers of an absorbent article 20. The indicia may be on, positioned on, formed on, formed with, printed on, or applied to either side, or both sides, of the one or more layers of an absorbent article 20. In some instances, suitable layers for indicia placement comprise one or more of a topsheet 24, a secondary topsheet, an acquisition layer 52, a distribution layer 54, a tissue layer 17, a core wrap 160, a bottom side 16′ of the core wrap 160, a top side 16 of the core wrap 160, and/or an additional layer positioned at least partially intermediate the topsheet 24 and the top side 16 of the core wrap 160 (hereafter sometimes referred to as “materials suitable for indicia placement”).

Either in addition to or separate from the indicia described above, any one or more of the suitable layers for indicia placement, or a portion thereof, may have a color different than any one or more of the remaining layers for indicia placement, or a portion thereof. The definition of the phrase “different color” above also applies to this part of the disclosure. In some instances, the indicia may be a different color than any one or more of the suitable layers for indicia placement.

Alternatively, an indicia may be on one of the suitable layers for indicia placement while another one of the remaining suitable layers for indicia placement may be a different color than the indicia. One example may be a blue indicia on a white tissue layer 17 with the acquisition layer 52 or topsheet 24 being teal.

In another example, a blue indicia may be on a white tissue layer 17 with the acquisition layer 52 and topsheet 24 also being white. As such, the blue indicia may be viewable from a wearer-facing surface.

In another example, a blue indicia may be on an acquisition layer 52, wherein the topsheet 24 and the acquisition layer 52 are simultaneously mechanically deformed and combined together, preferably nested together to provide a topsheet/acquisition layer laminate 245 having three-dimensional protrusions 250.

In an instance where the topsheet and the acquisition layer are simultaneously mechanically deformed and combined together, preferably nested together to provide a topsheet/acquisition layer laminate 245 having three-dimensional protrusions 250, the indicia may be applied to the acquisition layer 52 or the topsheet 24 before or after such mechanical deformation (or preferably namely nesting).

In an example, two different indicia may be positioned on the same or different layers for indicia placement. The two different indicia may be different in color, pattern, and/or graphic, for example. If the two different indicia are on different layers for indicia placement, the two layers may be the same color or different colors, or have portions that are the same color or different colors.

In some instances, a visible color of a portion of, or all of, the interior (wearer-facing surface) of an absorbent article 20 may be coordinated with and/or compliment a visible color of a portion of, or all of, the exterior (garment-facing surface) of the absorbent article 20, as described in further detail in U.S. Pat. No. 8,936,584. The indicia visible from the interior may also be coordinated with and/or compliment the indicia visible from the exterior of the absorbent article 20. In such an instance, the backsheet 25 of the absorbent article 20 may comprise an outer cover nonwoven and a backsheet film. The indicia visible from the exterior of the absorbent article 20 may be on the outer cover nonwoven or the backsheet film.

In still other instances, the visible indicia and/or color from the interior may also be coordinated with or compliment the indicia and/or color visible from the exterior of the absorbent article 20.

In addition to that described above, a first portion of one of the suitable layers for indicia placement may be a first color and a second portion of the same of the suitable layers for indicia placement may be a second color. The first and second colors may be a different color. In other instances, a first portion of one of the suitable layers for indicia placement may be a first color and a second portion of a different one of the suitable layers for indicia placement may be a second color. The first and second colors may be a different color.

In an instance, in an absorbent article 20, one of a topsheet 24, an acquisition layer 52, a portion of a core wrap 160, or an additional layer (e.g., a tissue layer 17) may be a different color than a different one of the topsheet 24, the acquisition layer 52, the portion of the core wrap 160, or the additional layer.

In another instance, in an absorbent article 20, one of a portion of a topsheet 24, a portion of an acquisition layer 52, a portion of a core wrap 160, or a portion of an additional layer may be a different color than a different one of the portion of the topsheet 24, the portion of the acquisition layer 52, the portion of the core wrap 160, or the portion of the additional layer.

In another instance, in an absorbent article 20, a first portion of one of a topsheet 24, an acquisition layer 52, a portion of a core wrap 160, or an additional layer may be a different color as a second portion of the same one of the topsheet 24, the acquisition layer 52, the core wrap 160, or the additional layer.

The process of the present disclosure may comprise applying the indicia to or positioning or printing the indicia on the topsheet 24, the acquisition layer 52, the tissue layer 17, a portion of the core wrap 160, and/or an additional layer positioned at least partially intermediate the topsheet 24 and the backsheet 25. The indicia may be positioned or printed on or applied to either side of the topsheet 24, the acquisition layer 52, the tissue layer 17, the portion of the core wrap 160, and/or the additional layer positioned at least partially intermediate the topsheet 24 and the backsheet 25. If the indicia is applied to or positioned or printed on the topsheet 24 or the acquisition layer 52, this step may be done before or after the topsheet 24 and the acquisition layer 52 are simultaneously mechanically deformed and combined together to provide the top sheet/acquisition layer laminate 245.

In some forms, the indicia may be positioned or printed on or applied to a tissue layer 17 that comprises pulp fibers. In other forms, the indicia may be positioned or printed on or applied to a garment-facing surface or a wearer-facing surface of the acquisition layer 52. In some instances, the materials suitable for indicia placement may be purchased with indicia thereon or the indicia may be applied to or printed or positioned on before or during feeding these materials into an absorbent article manufacturing line.

### Precursor Materials for the Topsheet and the Acquisition Layer

The three-dimensional substrate 240, and the topsheet/acquisition layer laminate 245 of the present invention can be made of any suitable nonwoven materials (“precursor materials”). In some cases, the three-dimensional substrate 240, and the topsheet/acquisition layer laminate 245 may also be free of cellulose materials. The precursor materials for the three-dimensional substrate 240, and the topsheet/acquisition layer laminate 245 may have suitable properties in order to be deformed. The suitable properties of the precursor materials may include: apparent elongation of the fibers, fiber mobility, ability to deform and stretch in the area where the three-dimensional protrusions 250 are formed. Hence, the precursor materials are capable of undergoing mechanical deformation to ensure that the three-dimensional protrusion 250 will not tend to recover or return to the prior configuration of a flat topsheet 24 laminated on a flat acquisition layer 52.

Several examples of nonwoven materials suitable for use as a three-dimensional substrate 240, or a topsheet 24 for the topsheet/acquisition layer laminate 245 may include, but are not limited to: spunbonded nonwovens; carded nonwovens; and nonwovens with relatively specific properties to be able to be readily deformed.

One suitable nonwoven material as a three-dimensional substrate 240, or a topsheet 24 for the topsheet/acquisition layer laminate 245 may be an extensible polypropylene/polyethylene spunbonded nonwoven. One suitable nonwoven material as a three-dimensional substrate 240, or a topsheet 24 for the topsheet/acquisition layer laminate 245 may be a spunbonded nonwoven comprising polypropylene and polyethylene. The fibers may comprise a blend of polypropylene and polyethylene. Alternatively, the fibers may comprise bi-component fibers, such as a sheath-core fiber with polyethylene on the sheath and polypropylene in the core of the fiber.

The three-dimensional substrate 240 may have a basis weight from 8 to 40 gsm or from 8 to 30 gsm or from 8 to 20 gsm. The topsheet 24 of the topsheet/acquisition layer laminate 245 may have a basis weight from 8 to 40 gsm or from 8 to 30 gsm or from 8 to 20 gsm.

Suitable nonwoven materials for the acquisition layer 52 may include, but are not limited to: spunbonded nonwovens, through-air bonded (“TAB”) carded high loft nonwoven materials, spunlace nonwovens, hydroentangled nonwovens, and resin bonded carded nonwoven materials. Spunbonded PET may be denser than carded nonwovens, providing more uniformity and opacity. Since PET fibers are not very extensible, the nonwoven can be bonded such that at least some of the fibers can be separated easily from the bond sites to allow the fibers to pull out of the bond sites and rearrange when the material is strained. This type of bonding, e.g. pressure bonding can help increasing the level of mobility of the fibers. Indeed, the fibers tend to pull out from the bond sites under tension.

The acquisition layer exhibits a basis weight from 10 to 120 gsm or from 10 to 100 gsm or from 10 to 80 gsm.

The topsheet 24 and/or acquisition layer 52 may have a density from 0.01 to 0.4 g/cm³ or from 0.01 to 0.25 g/cm³ or from 0.04 to 0.15 g/cm³.

The topsheet 24 and acquisition layer 52 may be joined together prior or during the mechanical deformation. If desired an adhesive, chemical bonding, resin or powder bonding, or thermal bonding between the topsheet 24 and acquisition layer 52 may be selectively utilized to bond certain regions or all of the topsheet 24 and acquisition layer 52 together. In addition, the topsheet 24 and acquisition layer 52 may be bonded during processing, for example, by carding the topsheet 24 of onto the acquisition layer 52 and thermal point bonding the combined layers.

Prior to any mechanical deformation, the topsheet 24 may be attached to the acquisition layer 52. For instance, the topsheet 24 may be attached to the acquisition layer 52 where the topsheet 24 and the acquisition layer 52 overlaps. The attachment of the topsheet 24 to the acquisition layer 52 may include a uniform continuous layer of adhesive, a discontinuous patterned application of adhesive or an array of separate lines, spirals, or spots of adhesive. The basis weight of the adhesive in the topsheet/acquisition layer laminate 245 may be from 0.5 to 30 gsm or from 1 to 10 gsm or from 2 to 5 gsm.

### Example

The topsheet and the acquisition layer were attached to each other with a hot melt adhesive applied in form of spirals with a basis weight of 5 gsm. The adhesive spirals were applied all across the cross machine direction on the topsheet surface facing the acquisition layer where the topsheet contacted the acquisition layer, for a length of 330 mm in the machine direction and started 65 mm from the topsheet front edge with respect to the machine direction. The acquisition layer was centered onto the topsheet with respect to the topsheet and placed 65 mm from the front MD edge of the topsheet. The topsheet and acquisition layer attached together form a composite web.

The topsheet and acquisition layer attached together have been simultaneously mechanically deformed by passing them between a pair of intermeshing male and female rolls. The topsheet of the topsheet/acquisition layer laminate was in contact with the male roll. The acquisition layer of the topsheet/acquisition layer laminate was in contact with the female roll. The teeth on the male roll have a rounded diamond shape like that shown in FIG. 16A, with vertical sidewalls and a radiused or rounded edge at the transition between the top and the sidewalls of the tooth. The teeth are 0.186 inch (4.72 mm) long and 0.125 inch (3.18 mm) wide with a CD spacing of 0.150 inch (3.81 mm) and an MD spacing of 0.346 inch (8.79 mm). The recesses in the mating female roll also have a rounded diamond shape, similar to that of the male roll, with a clearance between the rolls of 0.032-0.063 inch (0.813-1.6 mm). The process speed was 800 fpm and the depth of engagement (DOE) was 0.155 inch (3.94 mm), with the topsheet being in contact with the male roll and the acquisition layer being in contact with the female roll.

The topsheet of the topsheet/acquisition layer laminate was a hydrophilic coated mono component high elongation spunbond polypropylene (HES PP) nonwoven material with a density of 0.11 g/cm³. The mono component HES PP nonwoven material for the topsheet has an overall basis weight of 20 gsm. The mono component HES PP nonwoven material was first coated with a finish made of a fatty acid polyethylene glycol ester for the production of a permanent hydrophilic mono component HES PP nonwoven material. The topsheet of the topsheet/acquisition layer laminate had a width of 168 mm and a length of 488 mm.

The acquisition layer of the topsheet/acquisition layer laminate was an air through bonded nonwoven with a basis weight of 65 gsm with a density of 0.09 g/cm³. The acquisition layer comprises 4 denier coPET/PET (polyethylene terephthalate) bicomponent fibers which was treated with a surfactant. The acquisition layer of the topsheet/acquisition layer laminate had a width of 90 mm and a length of 330 mm.

The tissue layer was a wet-laid fibrous substrate made through the use of a patterned papermaking belt 300 for forming three-dimensionally structured wet-laid and wet-formed webs as described in U.S. Pat. No. 4,637,859, issued Jan. 20, 1987, to Trokhan. The basis weight of the tissue layer was 40 gsm with a Wet Burst Strength of 318 g according to the Wet Burst Strength Test Method and a total dry tensile strength of 2034 g/M measured according to the Total Dry Tensile strength Test Method. The tissue layer had a width of 105 mm and a length of 330 mm

### Diaper Prototypes

Diaper prototypes for the above example were produced using Pampers Active Fit S4 (size 4) diaper commercially available in Germany in November 2014. Pampers Active Fit S4 (size 4) diaper comprises a topsheet, an acquisition layer beneath the topsheet, a distribution layer beneath the acquisition layer, an absorbent core between the distribution and a backsheet beneath the absorbent core. Diaper prototypes for the above example were produced using Pampers Active Fit S4 (size 4) diaper.

The topsheet and acquisition layer attached together for the above example were placed on top of a tissue layer. The acquisition layer was located between the topsheet and the tissue layer. The topsheet/acquisition layer laminate with the tissue layer were placed on top of a Pampers Active Fit diaper commercially available in Germany in November 2014 from where the commercial topsheet and acquisition layer were removed while keeping the distribution layer in place. For each diaper prototype based on the above example, the topsheet/acquisition layer laminate were placed on top the tissue layer which was on top of the distribution layer with the three-dimensional protrusions protruding towards the backsheet.

First, the surface of tissue layer facing the distribution layer was attached to the distribution layer with a hot melt adhesive applied in form of spirals with a basis weight of 5 gsm such that the distribution layer is centered on the tissue layer with respect to the cross machine direction and starting at the distribution layer front edge with respect to the machine direction. The tissue layer is placed with regard to the distribution layer such as the front edge of the tissue layer was matching with the front edge of the distribution layer.

Secondly, the surface of the topsheet of the topsheet/acquisition layer laminate facing the distribution layer and the surface of the acquisition layer of the topsheet/acquisition layer laminate facing the distribution layer were attached to the distribution layer with a hot melt adhesive applied in form of spirals with a basis weight of 5 gsm, such that the topsheet/acquisition layer laminate is centered in the cross-machine direction. The front edge of the acquisition layer of the topsheet/acquisition layer laminate was matching with the front edge of the tissue layer and the front edge of the distribution layer.

The three-dimensional protrusions of the topsheet/acquisition layer laminate were protruding towards the backsheet because the topsheet of the topsheet/acquisition layer laminate was in contact with the male roll, as set out above.

Each prototype diaper was compacted in a bag at an In Bag Stack Height, i.e. the total caliper of 10 bi-folded diapers, of 90 mm for 1 week. Then the bag was opened and the diapers out of the bag were conditioned at least 24 hours prior to any testing at 23° C.+/−2° C. and 50%+/−10% Relative Humidity (RH).

### Test Methods

Unless otherwise specified, all tests described herein are conducted on samples that have been conditioned at a temperature of 23° C.±2° C. and a relative humidity of 50%±2% for a minimum of 2 hours prior to testing. All tests are conducted under the same environmental conditions. Samples conditioned as described herein are considered dry samples. Further, all tests are conducted in such conditioned room.

To obtain a sample of the tissue layer, use the following steps:
(1) Lay the absorbent article on a flat work surface and tape it down flat, elastics in a stretched state, wearer-facing surface facing the tester;
(2) Remove the three-dimensional substrate around a perimeter of the tissue layer using a razor blade and cryogenic spray (if needed);
(3) Remove any other layers intermediate the three-dimensional substrate and the tissue layer using a razor blade and cryogenic spray (if needed);
(4) Once the tissue layer is exposed, use the cryogenic spray to remove it from the absorbent article;
(5) Allow the sample to equilibrate at 23±2° C. and 50±5% relative humidity for 24 hours prior to testing; and
(6) Specimens that are appropriately sized for a particular test (within the knowledge of those of skill in the art) are cut from the tissue layer.

### Wet Burst Strength Test Method

The Wet Burst Strength as used herein is a measure of the ability of a fibrous structure to absorb energy, when wet and subjected to deformation with regard to the plane of the fibrous structure.

The wet burst strength of a fibrous structure (referred to as “sample” within this test method) is determined using an electronic burst tester and specified test conditions. The results obtained are averaged out of 4 experiments and the wet burst strength is reported for a fibrous structure 55 consisting of one single layer of wet-laid fibers.

### Equipment

Apparatus: Burst Tester—Thwing-Albert Vantage Burst Tester or equivalent ball burst instrument where the ball moves downward during testing. Refer to manufacturer's operation and set-up instructions. The ball diameter is 1.59 cm and the clamp opening diameter is 8.9 cm.
Calibration Weights—Refer to manufacturer's Calibration instructions
Conditioned Room Temperature and Humidity controlled within the following limits for

### Laboratory Testing:

Temperature: 23°±1° C.

Relative humidity: 50%±2%
Paper Cutter—Cutting board, 600 mm size
Scissors—100 mm, or larger
Pan—Approximate Width/Length/Depth: 240×300×50 mm, or equivalent
Distilled water at the temperature of the conditioned room used

### Sample Preparation

The fibrous structure 55 may be unwound from the roll.
The samples to be tested are conditioned in the conditioned room for 24 hours immediately before testing. All testing occurs within the conditioned room.
Cut the samples so that they are approximately 228 mm in length and width of approximately 140 mm in width.

### Operation

Set-up and calibrate the Burst Tester instrument according to the manufacturer's instructions for the instrument being used.
Holding the sample by the narrow edges, the center of the sample is dipped into a pan filled approximately 25 mm from the top with distilled water. The sample is left in the water for 4 (±0.5) seconds.
The excess water is drained from the sample for 3 (±0.5) seconds holding the sample in a vertical position.
The test should proceed immediately after the drain step. The sample should have no perforations, tears or imperfections in the area of the sample to be tested. If it does, start the test over.
The sample is placed between the upper and lower rings of the Burst Tester instrument. The sample is positioned centered and flat on the lower ring of the sample holding device in a manner such that no slack in the sample is present.
The upper ring of the pneumatic holding device is lowered to secure the sample.
The test is started. The test is over at sample failure (rupture) i.e., when the load falls 20 g from the peak force. The maximum force value is recorded.
The plunger will automatically reverse and return to its original starting position.
The upper ring is raised in order to remove and discard the tested sample.
The procedure is repeated until all replicates have been tested.

### Calculation

Wet Burst Strength=sum of peak load readings/number of replicates tested

Report the Wet Burst results to the nearest gram.

### Accelerated Compression Method

1. Cut 10 samples of the topsheet/acquisition layer laminate 245 (called herein specimen) to be tested and 11 samples of paper towel into a 3 inch×3 inch (7.6 cm×7.6 cm) square.
2. Measure the caliper of each of the 10 specimens at 2.1 kPa and a dwell time of 2 seconds using a Thwing-Albert ProGage Thickness Tester or equivalent with a 50-60 millimeter diameter circular foot. Record the pre-compression caliper to the nearest 0.01 mm
3. Alternate the layers of the specimens to be tested with the paper towels, starting and ending with the paper towels. The choice of paper towel does not matter and is present to prevent “nesting” of the protrusions in the deformed samples. The samples should be oriented so the edges of each of the specimens and each of the paper towels are relatively aligned, and the protrusions in the specimens are all oriented the same direction.
4. Place the stack of samples into a 40° C. oven and place a weight on top of the stack. The weight must be larger than the foot of the thickness tester. To simulate high pressures or low in-bag stack heights, apply 35 kPa (e.g. 17.5 kg weight over a 70×70 mm area). To simulate low pressures or high in-bag stack heights, apply 7 kPa (e.g. 3.5 kg weight over a 70×70 mm area).
5. Leave the samples in the oven for 15 hours. After the time period has elapsed, remove the weight from the samples and remove the samples from the oven.
6. Within 30 minutes of removing the samples from the oven, measure the post-compression caliper as directed in step 2 above, making sure to maintain the same order in which the pre-compression caliper was recorded. Record the post-compression caliper of each of the 10 specimens to the nearest 0.01 mm
7. Let the samples rest at 23±2° C. and at 50±2% relative humidity for 24 hours without any weight on them.
8. After 24 hours, measure the post-recovery caliper of each of the 10 specimens as directed in step 2 above, making sure to maintain the same order in which the pre-compression and post-compression calipers were recorded. Record the post-recovery caliper of each of the 10 specimens to the nearest 0.01 mm Calculate the amount of caliper recovery by subtracting the post-compression caliper from the post-recovery caliper and record to the nearest 0.01 mm
9. If desired, an average of the 10 specimens can be calculated for the pre-compression, post-compression and post-recovery calipers.

### Protrusion Base Width and Protrusion Height Test Methods

1) General Information

The Measured Protrusion Base Width and Measured Protrusion Height of the three-dimensional protrusions of the topsheet/acquisition layer laminate of an absorbent article are measured using a GFM Primos Optical Profiler instrument commercially available from GFMesstechnik GmbH, Warthestraβe 21, D14513 Teltow/Berlin, Germany. Alternative suitable non-touching surface topology profilers having similar principles of measurement and analysis, can also be used, here GFM Primos is exemplified.

The GFM Primos Optical Profiler instrument includes a compact optical measuring sensor based on a digital micro mirror projection, consisting of the following main components:
a) DMD projector with 800×600 direct digital controlled micro-mirrors
b) CCD camera with high resolution (640×480 pixels)
c) Projection optics adapted to a measuring area of at least 30×40 mm
d) Recording optics adapted to a measuring area of at least 30×40 mm
e) A table tripod based on a small hard stone plate
f) A cold light source (an appropriate unit is the KL 1500 LCD, Schott North America, Inc., Southbridge, Mass.)
g) A measuring, control, and evaluation computer running ODSCAD 6.3 software

Turn on the cold-light source. The settings on the cold-light source are set to provide a color temperature of at least 2800K.

Turn on the computer, monitor, and open the image acquisition/analysis software. In the Primos Optical Profiler instrument, select “Start Measurement” icon from the ODSCAD 6.3 task bar and then click the “Live Image button”.

The instrument is calibrated according to manufacturer's specifications using calibration plates for lateral (X-Y) and vertical (Z). Such Calibration is performed using a rigid solid plate of any non-shiny material having a length of 11 cm, a width of 8 cm and a height of 1 cm. This plate has a groove or machined channel having a rectangular cross-section, a length of 11 cm, a width of 6.000 mm and an exact depth of 2.940 mm. This groove is parallel to the plate length direction. After calibration, the instrument must be able to measure the width and depth dimensions of the groove to within ±0.004 mm.

All testing is performed in a conditioned room maintained at 23±2° C. and 50+/−10% relative humidity. The surface to be measured may be lightly sprayed with a very fine white powder spray. Preferably, the spray is NORD-TEST Developer U 89, available from Helling GmbH, Heidgraben, Germany.

2) Protrusion Base Width Test Method

The topsheet/acquisition layer laminate is extracted from the absorbent article by attaching the absorbent article to a flat surface in a taut planar (i.e. stretched planar) configuration with the topsheet of the topsheet/acquisition layer laminate facing up. Any leg or cuff elastics are severed in order to allow the absorbent article to lie flat. Using scissors, two longitudinal cuts are made through all layers above the absorbent core (i.e. the core wrap) along the edges of the topsheet/acquisition layer laminate. Two transversal cuts are made through the same layers following the front and back waist edges of the absorbent article.

The topsheet/acquisition layer laminate and any other layers above the absorbent core are then removed without perturbing the topsheet/acquisition layer laminate. Freeze spray (e.g. CRC Freeze Spray manufactured by CRC Industries, Inc. 885 Louis Drive, Warminster, Pa. 18974, USA), or equivalent aid may be used to facilitate removal of the uppermost layers from the absorbent article. The topsheet/acquisition layer laminate is then separated from any other layers, including any carrier layer (e.g. a nonwoven carrier layer, a tissue layer), using freeze spray if necessary. If a distribution layer, e.g. a pulp containing layer is attached to the topsheet/acquisition layer laminate, any residual cellulose fibers are carefully removed with tweezers without modifying the acquisition layer.

The topsheet/acquisition layer laminate with three-dimensional protrusions (conditioned at a temperature of 23° C.±2° C. and a relative humidity of 50%±10% for at least 24 hours) namely “the specimen” is laid down on a hard flat horizontal surface with the body-facing side upward, i.e. the topsheet of the topsheet/acquisition layer laminate being upward. Ensure that the specimen is lying in planar configuration, without being stretched, with the specimen uncovered.

A nominal external pressure of 1.86 kPa (0.27 psi) is then applied to the specimen. Such nominal external pressure is applied without interfering with the topology profile measurement. Such an external pressure is applied using a transparent, non-shining flat Plexiglas® plate 200 mm by 70 mm and appropriate thickness (approximately 5 mm) to achieve a weight of 83 g. The plate is gently placed on top of the specimen, such that the center point of the Plexiglas® plate is at least 40 mm away from any folds, with the entire plate resting on the specimen. A fold corresponds to a part of the absorbent article (e.g. the topsheet/acquisition layer laminate) where the absorbent article has been folded for packaging purposes.

Two 50 mm×70 mm metal weights each having a mass of 1200 g (approximate thickness of 43 mm) are gently placed on the Plexiglas® plate such that a 70 mm edge of each metal weight is aligned with the 70 mm edges of the Plexiglas® plate. A metal frame having external dimensions of 70 mm×80 mm and interior dimensions of 42 mm×61 mm, and a total weight of 142 g (approximate thickness 6 mm), is positioned in the center of the Plexiglas® plate between the two end weights with the longest sides of the frame aligned with the longest sides of the plate.

If the specimen is smaller than 70×200 mm, or if a large enough area without a fold is not present, or if an area of interest is close to the edges of the specimen and can't be analyzed with the Plexiglas and weights settings described above, then the X-Y dimensions of the Plexiglas® plate and the added metal weights may be adjusted to reach a nominal external pressure of 1.86 kPa (0.27 psi) while maintaining a minimum 30×40 mm field of view. At least 10 complete three-dimensional protrusions of the specimen should be captured in the field of view of 30 mm×40 mm.

Position the projection head to be normal to the specimen surface (i.e. to the topsheet of the topsheet/acquisition layer laminate).
Adjust the distance between the specimen and the projection head for best focus.
In the Primos Optical Profiler instrument, turn on the button “Pattern” to make a red cross appear on the screen ross and a black cross appears on the specimen.
Adjust the focus control until the black cross is aligned with the red cross on the screen.
Adjust image brightness then capture a digitized image.
In the Primos Optical Profiler instrument, change the aperture on the lens through the hole in the side of the projector head and/or altering the camera “gain” setting on the screen.
When the illumination is optimum, the red circle at the bottom of the screen labeled “I.O.” will turn green.
Click on the “Measure” button.

The topology of the upper surface of the topsheet/acquisition layer laminate specimen is measured through the Plexiglas plate over the entire field of view 30 mm×40 mm. It is important to keep the specimen still stationary during this time in order to avoid blurring of the captured image. The image should be captured within the 30 seconds following the placement of the Plexiglas plate, metal weights and frame on top of the specimen.

After the image has been captured, the X-Y-Z coordinates of every pixel of the 40 mm×30 mm field of view area are recorded. The X direction is the direction parallel to the longest edge of the rectangular field of view, the Y direction is the direction parallel to the shortest edge of the rectangular field of view. The Z direction is the direction perpendicular to the X-Y plane. The X-Y plane is horizontal while the Z direction is vertical, i.e. orthogonal to the X-Y plane.

These data are smoothed and filtered using a polynomial filter (n=6), a median filter 11 pixels by 11 pixels, and a structure filter 81 pixels by 81 pixels. The polynomial filter (n=6) approximates the X-Y-Z coordinate surface with a polynomial of order 6 and returns the difference to the approximated polynomial. The median filter 11 pixels by 11 pixels divides the field of view (40 mm×30 mm) in X-Y squares of 11 pixels by 11 pixels. The Z coordinate of the pixel located at the center of a given 11 pixels by 11 pixels square will be replaced by the mean Z value of all the pixels of this given square. The structure filter 81 pixels by 81 pixels, removes the waviness of the structure and translates all the Z peak values belonging to the bottom surface of the Plexiglas plate to a top X-Y plane.

A Reference Plane is then defined as the X-Y plane intercepting the surface topology profile of the entire field of view (i.e. 30 mm×40 mm), 100 microns below this top X-Y plane. In the Primos Optical Profiler instrument, to measure the Material Area of the Reference Plane (Z=−0.1 mm), click on the button “Evaluate”. Then, apply a pre-filtering routine including a polynomial filter (n=6), a median filter 11 by 11 and a structure filter (n=81) using the function “Filter”. Save the image to a computer file with “.omc” extension.

The same above procedure is then executed on the topsheet/acquisition layer laminate with the garment-facing side upward (i.e. the acquisition layer of the topsheet/acquisition layer laminate being upward), the 40 mm×30 mm field of view being located at the exact same X-Y position of the topsheet/acquisition layer laminate.

The Empty Area of the reference plane can be defined as the area of the Reference Plane that is above the surface profile. The Empty Areas having boundaries strictly located inside the field of view area (i.e. 30 mm×40 mm) without crossing or overlapping with the boundaries of the field of view area (i.e. 40 mm×30 mm) are defined as Isolated Empty Area(s). The Measured Protrusion Base Width is defined for an Isolated Empty Area as the diameter of the biggest circle that can be inscribed inside a given Isolated Empty Area. This circle should only overlap with the Isolated Empty Area.

In the Primos Optical Profiler instrument, this can be done by clicking on “Draw circle” and drawing the biggest inscribed circle possible in a chosen Isolated Empty Area. Click on “Show sectional picture”, the circle diameter can be measure via clicking on the extremity of the sectional picture profile and then clicking on “Horizontal distance” to obtain the Protrusion Base Width.

For both of the acquired and digitized images, the Protrusion Base Width of all the Isolated Empty Areas is determined. Then, the Measured Protrusion Base Width is calculated as the arithmetic average of the 6 biggest Protrusion Base Widths.

3) Protrusion Height Test Method

The topsheet/acquisition layer laminate is extracted from the absorbent article as described above in the Protrusion Base Width Test Method.

The topsheet/acquisition layer laminate specimen comprising three-dimensional protrusions is then conditioned and scanned under a pressure of 1.86 kPa (0.27 psi) with the body-facing side upward, i.e. the topsheet of the topsheet/acquisition layer laminate being upward as described above in the Protrusion Base Width Test Method.

After the image has been captured, the X-Y-Z coordinates of every pixel of the 40 mm×30 mm field of view area are recorded and smoothed/filtered as described above in the Protrusion Base Width Test Method. A reference plane is also defined as described above in the Protrusion Base Width Test Method.

In the Primos Optical Profiler instrument, to measure the Material Area of the Reference Plane (Z=−0.1 mm), click on the button “Evaluate”. Then apply a pre-filtering routine including a polynomial filter (n=6), a median filter 11 by 11 and a structure filter (n=81) using the function “Filter”. Save the image to a computer file with “.omc” extension.

The same above procedure set out in the Protrusion Base Width Test Method is then executed on the topsheet/acquisition layer laminate with the garment-facing side upward (i.e. the acquisition layer of the topsheet/acquisition layer laminate being upward), the 40 mm×30 mm field of view being located at the exact same X-Y position of the topsheet/acquisition layer laminate.

The Empty Area of the reference plane can be defined as the area of the Reference Plane that is above the surface profile. The Empty Area having boundaries strictly located inside the field of view area (i.e. 30 mm×40 mm) without crossing or overlapping with the boundaries of the field of view area (i.e. 40 mm×30 mm) are defined as Isolated Empty Area(s). The Protrusion Height is defined for an Isolated Empty Area as the distance between the minimum Z value of the points of the topsheet/acquisition layer laminate surface profile having X-Y coordinates located in this Isolated Empty Area, and the Z value of the top X-Y plane.

Click on “Draw N parallel lines” and draw a first segment parallel to the X axis of the field of view (direction of the longest dimension of the field of view) passing through the center of the Isolated Empty Area and extending outside the Isolated Empty Area boundaries. The center of the Isolated Empty Area corresponds to the middle of the segment parallel to the Y axis of the field of view and joining the biggest and smallest Y value of the Isolated Empty Area. Then input the “number” of lines to be drawn and set the “distance” between lines to 0.05 mm Enough lines need to be drawn such to cover the entire Isolated Empty Area. Leave the averaging parameter to 0 then click “Ok”. Then click on “Show sectional picture”. Click on the point of the sectional picture profile having the minimum Z value and click on “Vertical distance” to obtain the Protrusion Height.

For both of the acquired and digitized images, the Protrusion Height of all the Isolated Empty Areas is determined. Then, the Measured Protrusion Height is calculated as the arithmetic average of the 6 biggest Protrusion Heights.

### Height Test Methods for a Three-Dimensional Substrate

1) Sample Preparation

Take the steel frame and place double-sided adhesive tape on the bottom surface surrounding the interior opening. To obtain a specimen, tape the absorbent article to a rigid flat surface in a planar configuration with the body-facing surface up (i.e. the three-dimensional substrate). Any leg elastics may be cut to facilitate laying the article flat. Remove the release paper of the tape, and adhere the steel frame to the three-dimensional substrate of the absorbent article. Using a razor blade, excise the three-dimensional substrate from the underling layers of the absorbent article around the outer perimeter of the frame. Carefully remove the topsheet specimen such that its longitudinal and lateral extension is maintained. A cryogenic spray (such as Cyto-Freeze, Control Company, Houston Tex.) can be used to remove the three-dimensional substrate specimen from the underling layers, if necessary. Five replicates obtained from five substantially similar absorbent articles are prepared for analysis. Precondition the specimens at about 23° C.±2° C. and about 50%±2% relative humidity for 2 hours prior to testing.

2) Surface Topography Image

Surface height measurements are performed on a three-dimensional surface topography image obtained using an optical 3D surface topography measurement system. A suitable optical 3D surface topography measurement system is the GFM MikroCAD Premium instrument commercially available from GFMesstechnik GmbH, Teltow/Berlin, Germany.

The system includes the following main components:
a) a Digital Light Processing (DLP) projector with direct digital controlled micro-mirrors;
b) a CCD camera with at least a 1600×1200 pixel resolution;
c) projection optics adapted to a measuring area of at least 60 mm×45 mm;
d) recording optics adapted to a measuring area of 60 mm×45 mm;
e) a table tripod based on a small hard stone plate;
f) a blue LED light source;
g) a measuring, control, and evaluation computer running surface topography analysis software (suitable software is ODSCAD software version 6.2 available from GFMesstechnik GmbH, Teltow/Berlin, Germany); and
h) calibration plates for lateral (x-y) and vertical (z) calibration available from the vendor.

The optical 3D surface topography measurement system measures the surface height of a specimen using the digital micro-mirror pattern fringe projection technique. The result of the analysis is a map of surface height (z-directional or z-axis) versus displacement in the x-y plane. The system has a field of view of 60×45 mm with an x-y pixel resolution of approximately 40 microns. The height resolution is set at 0.5 micron/count, with a height range of +/−15 mm. All testing is performed in a conditioned room maintained at about 23±2° C. and about 50±2% relative humidity.

Calibrate the instrument according to manufacturer's specifications using the calibration plates for lateral (x-y axis) and vertical (z axis) available from the vendor.

Place the specimen and frame on the table beneath the camera. Center the specimen within the camera field of view, so that only the body-facing surface of the specimen surface is visible in the preview image.

Collect a height image (z-direction) of the specimen by following the instrument manufacturer's recommended measurement procedures, which may include, focusing the measurement system and performing a brightness adjustment. No pre-filtering options should be utilized. Save the collected height image file.

Load the height image into the surface analysis portion of the software. The following filtering procedure is then performed on each image: 1) removal of invalid points; 2) removal of peaks (small localized elevations); 3) polynomial filtering of the material part with a rank of n=5, with exclusion of 30% of the peaks and 30% of the valleys from the material part, and 5 cycles.

3) Projection Height Test Method
1. Draw a line connecting the top peaks of two adjacent protrusions, with the line crossing a land area located between them
2. Generate a sectional image of the height image along the drawn line. Along the sectional line, measure the vertical height (z-direction) difference between the top peak of the protrusion and the adjacent valley of the land area. Record the height to the nearest 0.1 μm; and
3. Repeat the measurement for 10 different protrusions. Average 10 height measures and report this value to the nearest 0.1 μm. This is the Z-directional height of the protrusions of the three-dimensional substrate.

4) Overall Substrate Height Test Method
1. Draw a line connecting the top peaks of two adjacent protrusions, with the line crossing the center of a recess located between each of the protrusions and within a recess;
2. Generate a sectional image of the height image along the drawn line. Along the sectional line, measure the vertical height difference between the top peak of the protrusion and the adjacent base of the recess. Record the height to the nearest 0.1 μm; and
3. Repeat protrusion top peak to base of recess height measures for 10 different protrusions. Average together 10 measures and report this value to the nearest 0.1 μm. This is the overall Z-directional height of the three-dimensional substrate.

5) Recess Height Test Method
1. Draw a line connecting the base of two adjacent recesses, with the line crossing a land area located between each of the recesses;
2. Generate a sectional image of the height image along the drawn line. Along the sectional line, measure the vertical height (z-direction) difference between the base of the recess and the adjacent valley of the land area. Record the height to the nearest 0.1 μm;
3. Repeat measurement for 10 different recesses. Average 10 measures and report this value to the nearest 0.1 μm. This is the Z-directional height of the recesses of the three-dimensional substrate.

When the pattern configuration does not allow measuring one of the z-directional heights described above, it can be calculated from the equation: Overall Substrate Height=Protrusion Height+Recess Height.

In cases where the three-dimensional substrate only comprises a plurality of protrusions and recesses without any land areas or plane P, the above Dry Caliper measurement Test Method at 0.1 kPa shall be applied.

### Basis Weight Test Method

Basis weight of a wet-laid three-dimensional fibrous substrate is measured on stacks of twelve usable units using a top loading analytical balance with a resolution of ±0.001 g. The balance is protected from air drafts and other disturbances using a draft shield. A precision cutting die, measuring 3.500 in ±0.0035 in by 3.500 in ±0.0035 in is used to prepare all samples.

With a precision cutting die, cut the samples into squares. Combine the cut squares to form a stack twelve samples thick. Measure the mass of the sample stack and record the result to the nearest 0.001 g.

The Basis Weight is calculated in lbs/3000 ft² or g/m² as follows:

Basis Weight=(Mass of stack)/[(Area of 1 square in stack)×(Number of squares in stack)]

For example,

Basis Weight (lbs/3000 ft²)=[[Mass of stack (g)/453.6 (g/lbs)]/[12.25 (in²)/144 (in²/ft²)×12]]×3000

or,

Basis Weight (g/m²)=Mass of stack (g)/[79.032 (cm²)/10,000 (cm²/m²)×12]

Report result to the nearest 0.1 lbs/3000 ft² or 0.1 g/m². Sample dimensions can be changed or varied using a similar precision cutter as mentioned above, so as at least 100 square inches of sample area in stack.

### Average Density Test Method

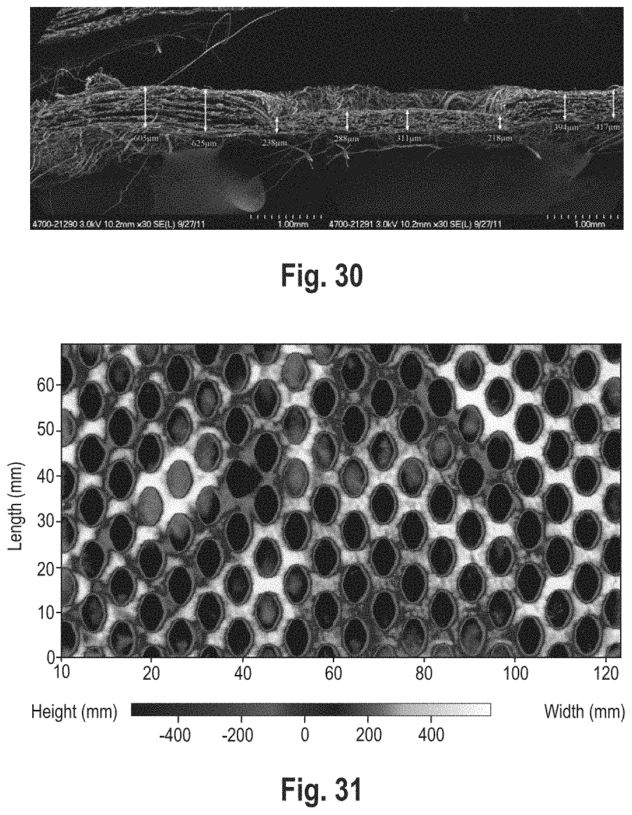

A wet-laid three-dimensional fibrous substrate comprises network regions and pluralities of discrete zones which have characteristic densities. A cross-sectional, SEM micrograph of such a fibrous substrate is shown in FIG. 30. The regions of the wet-laid three-dimensional fibrous substrate are illustrated in the micrograph by the zones comprising different caliper or thickness. These caliper differences are one of the factors responsible for the superior performance characteristics of these fibrous substrates.

The regions with higher caliper are lower in structure density and these are typically referred to as “pillows”. The regions with lower caliper are higher in structure density and these are typically referred to as “knuckles.”

The density of the regions within a wet-laid three-dimensional fibrous substrate are measured by first cutting for a length of at least 2-3 knuckle and pillow regions with a previously unused single edge PTFE-treated razor blade such as the GEM® razor blades available from Ted Pella Inc. Only one cut is made per razor blade. Each cross-sectioned sample is mounted on a SEM sample holder, secured by carbon paste, then plunged and frozen in liquid nitrogen. The sample is transferred to an SEM chamber at −90° C., coated with Gold/Palladium for 60 seconds and analyzed using a commercially available SEM equipped with a cryo-system such as a Hitachi S-4700 with Alto cryo system and PCI (Passive Capture Imaging) software for image analysis or an equivalent SEM system and equivalent software. All samples are evaluated while frozen to ensure their original size and shape under vacuum while in the scanning electron microscope.

Pillow and knuckle thickness or network regions and discrete zone thickness are determined using image analysis software associated with the SEM equipment. As the measurements are the thickness of a sample, such analysis software is standard for all SEM equipment. Measurements are taken where the thickness of the region or zone are at their respective local maximum values. Thickness values for at least 2 individual, separate network regions and at least 2 individual, discrete zones are recorded and then averaged and reported as the average network region thickness and average discrete zone thickness, respectively. The average thicknesses are measured in units of microns.

Separately, the basis weight of the sample being measured for density is determined using the Basis Weight Test Method defined herein. The basis weight as measured in gsm (g/m²) is calculated using the Basis Weight Test Method and used to calculate the region density.

Below is an example for calculating the average network density and average discrete zone density for a sample with a basis weight of 100 g/m², a network region average thickness of 625 micron, and a discrete zone average thickness of 311 micron.

$$Average network density ( \frac{g}{cc} ) = \frac{basis weight}{network thickness} = \frac{100 \frac{g}{{m}^{2}}}{625 \times {10}^{- 6} m} \times \frac{{m}^{2}}{1 \times {10}^{6} cc} = 0.16 \frac{g}{cc}$$

$$Average discrete zone density ( \frac{g}{cc} ) = \frac{basis weight}{discrete zone thickness} = \frac{100 \frac{g}{{m}^{2}}}{311 \times {10}^{- 6} m} \times \frac{{m}^{2}}{1 \times {10}^{6} cc} = 0.32 \frac{g}{cc}$$

### Tensile Test Method: Elongation, Tensile Strength, TEA and Modulus

Elongation, Tensile Strength, TEA and Tangent Modulus are measured on a constant rate of extension tensile tester with computer interface (a suitable instrument is the EJA Vantage from the Thwing-Albert Instrument Co. Wet Berlin, N.J.) using a load cell for which the forces measured are within 10% to 90% of the limit of the cell. Both the movable (upper) and stationary (lower) pneumatic jaws are fitted with smooth stainless steel faced grips, 25.4 mm in height and wider than the width of the test specimen. An air pressure of about 60 psi is supplied to the jaws.

Eight usable units of fibrous substrate are divided into two stacks of four samples each. The samples in each stack are consistently oriented with respect to machine direction (MD) and cross direction (CD). One of the stacks is designated for testing in the MD and the other for CD. Using a one inch precision cutter (Thwing Albert JDC-1-10, or similar) cut 4 MD strips from one stack, and 4 CD strips from the other, with dimensions of 1.00 in ±0.01 in wide by 3.0-4.0 in long. Each strip of one usable unit thick will be treated as a unitary specimen for testing.

Program the tensile tester to perform an extension test, collecting force and extension data at an acquisition rate of 20 Hz as the crosshead raises at a rate of 2.00 in/min (5.08 cm/min) until the specimen breaks. The break sensitivity is set to 80%, i.e., the test is terminated when the measured force drops to 20% of the maximum peak force, after which the crosshead is returned to its original position.

Set the gauge length to 1.00 inch. Zero the crosshead and load cell. \Insert at least 1.0 in of the unitary specimen into the upper grip, aligning it vertically within the upper and lower jaws and close the upper grips. Insert the unitary specimen into the lower grips and close. The unitary specimen should be under enough tension to eliminate any slack, but less than 5.0 g of force on the load cell. Start the tensile tester and data collection. Repeat testing in like fashion for all four CD and four MD unitary specimens.

Program the software to calculate the following from the constructed force (g) verses extension (in) curve:

Tensile Strength is the maximum peak force (g) divided by the sample width (in) and reported as Win to the nearest 1 g/in.

Adjusted Gauge Length is calculated as the extension measured at 3.0 g of force (in) added to the original gauge length (in).

Elongation is calculated as the extension at maximum peak force (in) divided by the Adjusted Gauge Length (in) multiplied by 100 and reported as % to the nearest 0.1%

Total Energy (TEA) is calculated as the area under the force curve integrated from zero extension to the extension at the maximum peak force (g*in), divided by the product of the adjusted Gauge Length (in) and specimen width (in) and is reported out to the nearest 1 g*in/in².

Replot the force (g) verses extension (in) curve as a force (g) verses strain curve. Strain is herein defined as the extension (in) divided by the Adjusted Gauge Length (in).

Program the software to calculate the following from the constructed force (g) verses strain curve:

Tangent Modulus is calculated as the slope of the linear line drawn between the two data points on the force (g) versus strain curve, where one of the data points used is the first data point recorded after 28 g force, and the other data point used is the first data point recorded after 48 g force. This slope is then divided by the specimen width (2.54 cm) and reported to the nearest 1 g/cm.

The Tensile Strength (g/in), Elongation (%), Total Energy (g*in/in²) and Tangent Modulus (g/cm) are calculated for the four CD unitary specimens and the four MD unitary specimens. Calculate an average for each parameter separately for the CD and MD specimens.

### Calculations:

Geometric Mean Tensile=Square Root of [MD Tensile Strength (g/in)×CD Tensile Strength (g/in)]

Geometric Mean Peak Elongation=Square Root of [MD Elongation (%)×CD Elongation (%)]

Geometric Mean TEA=Square Root of [MD TEA (g*in/in²)×CD TEA (g*in/in²)]

Geometric Mean Modulus=Square Root of [MD Modulus (g/cm)×CD Modulus (g/cm)]

Total Dry Tensile Strength (TDT)=MD Tensile Strength (g/in)+CD Tensile Strength (g/in)

Total TEA=MD TEA (g*in/in²)+CD TEA (g*in/in²)

Total Modulus=MD Modulus (g/cm)+CD Modulus (g/cm)

Tensile Ratio=MD Tensile Strength (g/in)/CD Tensile Strength (g/in)

### Topographic Measurements of Differential Density Fibrous Substrates Test Method

Topographic measurements of differential density fibrous substrates are obtained via computer-controlled fringe-projection optical profilometry. Optical profilometer systems measure the physical dimensions of the test surface, resulting in a map of surface height elevation (z), versus lateral displacement in the x-y plane. A suitable optical profilometer instrument will have a field of view and x-y resolution such that the acquired images possess at least 10 pixels linearly across the narrowest feature being measured. A suitable instrument is a GFM Mikrocad system, running ODSCAD software version 4 or 6, or equivalent, available from GFMessthechnik GmbH, Teltow, Germany.

If necessary, in order to make samples suitably reflective for accurate measurement of the surface features, the surface to be measured is lightly sprayed with a very fine white powder spray. Preferably this spray is NORD-TEST Developer U 89, available from Helling GmbH, Heidgraben, Germany, which is sold for the detection of cracks in metal objects and welds. Samples should be equilibrated at 23° C.±2° C. and 50%±2% relative humidity for at least 2 hours immediately prior to applying such a spray, and for at least 2 hours after spraying. Care is taken to deposit only the minimum amount of white spray needed to create a thin reflective white coating.

Samples should be equilibrated at 23° C.±2° C. and 50%±2% relative humidity for at least 2 hours immediately prior to acquiring measurements.

The area of the wet-laid three-dimensional fibrous substrates to be measured is restricted solely to areas possessing regions with different densities and excluding other areas or zones that might be present. The sample is placed with the surface area to be measured facing upward, underneath and normal to, the profilometer's projection head. The instrument manufacturer's instructions are followed, and optimized illumination and reflection requirements are achieved as outlined by the manufacturer. Digital images are then captured and stored.

Any portion of the image that is not part of the area to be measured should be cropped out of the captured image. Such cropping must occur prior to any further image processing, filtering or measurement analysis. The size of the resultant cropped image may vary between samples and images, depending upon the dimensions of the patterned area of that sample.

Prior to making measurements, the images are processed in the instrument software, in order to lightly smooth noise in the images, and to reduce irregularity or waviness due to the sample's overall shape. This noise filtering processing includes the removal of invalid pixel values (those black pixels having a grey value at the dark limit of the grayscale range), and the removal of spike values or outlier peaks (those very bright pixels identified by the software as statistical outliers). A polynomial high-pass filter is then utilized with settings of: n=8, difference. For samples with very small features where it is difficult to clearly observe the pattern features, it may be useful to also apply a Fourier filter (for example: a 5 mm wave filter, fine structure result). When such a Fourier filter is used, it removes features larger than the filter length as noise, and consequently reduces variability, lowering the statistical standard deviation around the topography measurements. It is therefore essential that the size of the filter used is larger than any features of interest so as not to filter out said features. Processed images such as the topography image shown in FIG. 31, can be displayed, analyzed and measured. FIG. 31 was cropped then flattened via filtering with a polynomial (n=8 difference) filter to remove irregularity due to the sample's overall waviness.

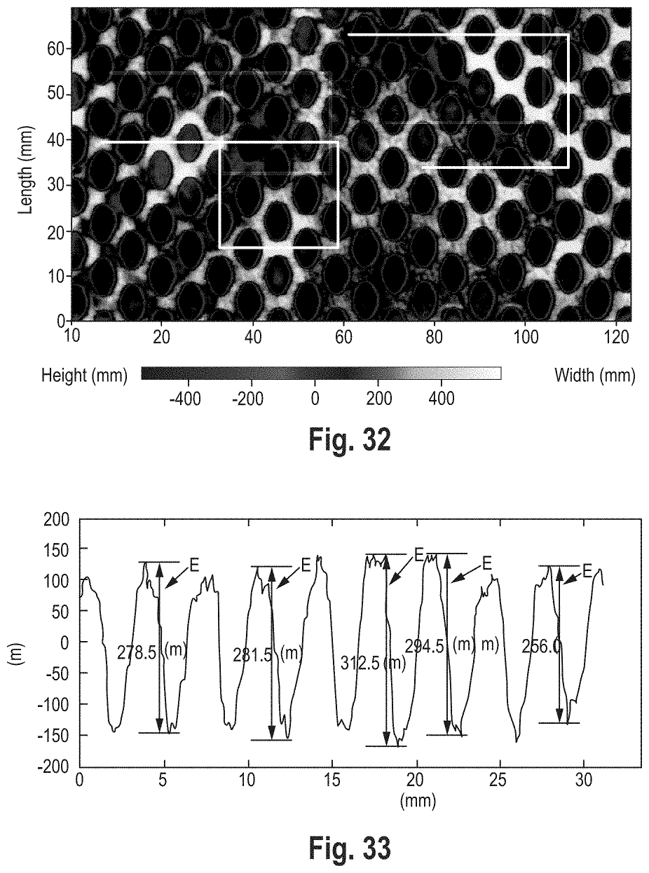

Measurements are then made from the processed topography images to yield the spatial parameters of elevation differential (E), and transition region width (T). These measurements are achieved by using the instrument software to draw straight line regions of interest within a topography image of the sample's x-y surface, and to then generate height profile plots along these straight lines. The straight line regions of interest are drawn such that they sample several different locations within each image, crossing continuous regions and the center of adjacent discrete zones. The lines are drawn so that they bisect each transition region between continuous and discrete zones at an angle perpendicular to the long axis of the transition region, as shown in FIG. 32. As shown in FIG. 32, a series of straight line regions of interest, drawn across the continuous and discrete zones, bisecting each transition region at an angle perpendicular to the long axis of the transition region. The parameters (E) and (T) are then measured from the height profile plots generated from these straight line regions of interest.

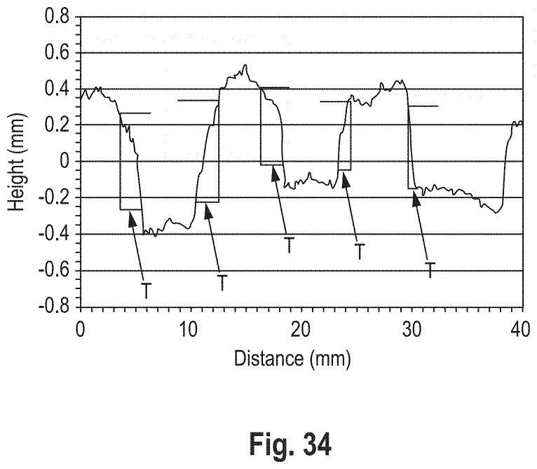

In a height profile plot, the plot's x-axis represents the length of the line, and the y-axis represents the vertical elevation of the surface perpendicular to the sample's planar surface. The elevation differential (E) is measured in micrometers as the vertical straight-line distance from the apex of a peak to the lowest point of an adjacent recess, on a height profile plot as shown in FIG. 33. As illustrated in FIG. 33, the height profile plot along a straight line region of interest, drawn through a topography image, shows several elevation differential (E) measurements. Typically this represents the maximum vertical elevation differential between the surface of a continuous region and an adjacent discrete zone, or vice versa. The transition region width (T) is measured in micrometers as the x-axis width of the curve across the central sixty percent (60%) of the elevation differential (E), on a height profile plot as shown in FIG. 34. As illustrated in FIG. 34, the height profile plot along a straight line region of interest, drawn through a topography image, shows several transition region widths (T). Typically, this represents the rate of transition from a continuous region to an adjacent discrete zone, or vice versa.

Where a sample has discrete zones which appear to fall into two or more distinct classes, as determined by visually observing their overall shape, size, elevation, and density, then separate values of (E) and (T) are to be determined for each discrete zone class and adjacent continuous region pairing.

If the sample visibly appears to have more than one pattern of discrete zones in different locations on the product, then each pattern is to have its values of (E) and (T) determined separately from the other pattern(s).

If a sample has a first region and an adjacent second region, wherein the first and second regions visibly appear to differ in their surface elevation, then the product is to have values of (E) and (T) measured from these regions. In this case all the method instructions given herein are to be followed and the first and second regions substituted for both the continuous region and the discrete zones named in this method.

For each pattern to be tested, five replicate product samples are imaged, and from each replicate sample measurements are made of at least ten elevation differentials (E) for each class of discrete zone, and ten transition region widths (T) for each class of discrete zone. This is repeated for each planar surface of each sample. Values of (E) and (T) are reported from the planar surface possessing the largest value of (E). For each parameter calculated for a specific pattern and discrete zone class, the values from each of the five replicate samples are averaged together to give the final value for each parameter.

### Dry Caliper Test Method

The intent of this method is to provide a procedure to determine the dry caliper for each layer of the tissue layer 17 under predefined pressure. The test can be executed with a conventional caliper micrometer, such as Type DM 2000 available from Wolf-Messtechnik GmbH, Am St. Niclas Schacht 13, Freiberg (Germany), having a circular sample foot of 15 mm diameter, having a weight for the foot of 17.2 g and additional weights of 20.0 g or 69.6 g or 106.9 g in order to achieve a total of 37.2 g or 86.8 g or 124.1 g to adjust the pressure to 2.065 kPa or 4.819 kPa or 6.889 kPa respectively (equivalent to 0.3 psi or 0.7 psi or 1.0 psi).

The caliper of each layer of the tissue layer 17 is determined. The total caliper of the distribution material is the sum of the caliper of each layer of fibrous substrate of the distribution material.

The Dry Caliper measurement is carried out on the following square samples: of 3 cm centered on one single layer of the distribution material to obtain the caliper of one layer.

### Basic Protocol for Dry Caliper

1. The distribution material is allowed to equilibrate at 23+−1 deg. C. and 50+−2% relative humidity for 8 hours.
2. The center of the sample is determined as described above and marked on the wearer surface of the sample.
3. The sample is positioned under the caliper gauge with the wearer surface toward the sample contact foot and with the center of the sample centered under the foot.
5. The sample contact foot is gently lowered into contact with the surface of the sample.
6. A Pressure of 2.06 kPa (0.3 psi) or 4.819 kPa (0.7 psi) or 6.889 kPa (1.0 psi) is applied.
7. The caliper reading is taken 2 seconds after the foot comes into contact with the sample.

The caliper is the average of three replicates and is reported in millimeters rounded to the nearest 0.01 mm.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article having a longitudinal axis and a transverse axis generally perpendicular to the longitudinal axis, the absorbent article further comprising:
a topsheet;
a backsheet;
an acquisition layer disposed between the topsheet and the backsheet, wherein the topsheet and the acquisition layer form a three dimensional substrate comprising a first surface and an opposing second surface, a plurality of recesses and a plurality of land areas, wherein each recess is separated from one another by at least one land area, wherein each of the plurality of recesses comprises an opening in the first surface at a base of the recess, side walls extending from the second surface to a distal portion;
a first absorbent layer disposed between the acquisition layer and the backsheet, the first absorbent layer comprising a mixture of pulp fibers and synthetic fibers; and
a second absorbent layer disposed between the first absorbent layer and the backsheet, the second absorbent layer comprising a core wrap and super absorbent polymer attached to the core wrap.

2. The absorbent article of claim 1, wherein the synthetic fibers comprise bicomponent fibers.

3. The absorbent article of claim 2, wherein the first absorbent layer is combined with the topsheet and acquisition layer to form the three dimensional substrate such that the first absorbent layer forms at least a portion of the second surface, side walls, and distal portions of the plurality of recesses.

4. The absorbent article of claim 2, wherein the bicomponent fibers comprise polyethylene terephthalate.

5. The absorbent article of claim 3, wherein the bicomponent fibers comprise polyethylene terephthalate.

6. The absorbent article of claim 1, wherein the acquisition layer comprises bicomponent fibers.

7. The absorbent article of claim 1, wherein the acquisition layer is a spunlace nonwoven.

8. The absorbent article of claim 1, wherein the acquisition layer has a basis weight of between about 10 gsm to about 80 gsm.

9. The absorbent article of claim 1, wherein the acquisition layer is a carded nonwoven.

10. The absorbent article of claim 1, wherein the topsheet is a nonwoven material.

11. The absorbent article of claim 10, wherein the topsheet is a spunbond nonwoven.

12. The absorbent article of claim 1, comprising a longitudinal axis, and a transversal axis perpendicular to the longitudinal axis, wherein a width of the acquisition layer in a direction parallel to the transversal axis is less than a width of the topsheet in a direction parallel to the transversal axis.

13. The absorbent article of claim 11, wherein the topsheet comprises a blend of polypropylene and polyethylene fibers.
